(19) United States
(12) Patent Application Publication
(10) Pub. No.: US 2023/0200685 A1
(43) Pub. Date: Jun. 29, 2023

(54) FLUID CHARACTERISTIC SENSOR

(71) Applicant: Murata Manufacturing Co., Ltd., Nagaokakyo-shi (JP)

(72) Inventors: Takafumi MORIASA, Nagaokakyo-shi (JP); Shuichi KAWATA, Nagaokakyo-shi (JP); Mika TAKADA, Nagaokakyo-shi (JP)

(21) Appl. No.: 18/116,007

(22) Filed: Mar. 1, 2023

Related U.S. Application Data
(63) Continuation of application No. PCT/JP2021/034817, filed on Sep. 22, 2021.

(30) Foreign Application Priority Data
Sep. 28, 2020 (JP) 2020-162353

(51) Int. Cl. A61B 5/145; A61B 5/15

(52) U.S. Cl. CPC A61B 5/14507; A61B 5/150099

(57) ABSTRACT

A fluid characteristic sensor includes a pressure loss generator to generate a pressure loss when the fluid flows, a first flow path connected to the pressure loss generator and through which the fluid and a working liquid that is a polar solvent flow, a partition wall provided in the first flow path to partition the fluid and the working liquid from each other, and a potential measurer connected to the first flow path to measure a flow potential generated when the working liquid flows.

### CROSS REFERENCE TO RELATED APPLICATIONS

This application claims the benefit of priority to Japanese Patent Application No. 2020-162353 filed on Sep. 28, 2020 and is a Continuation Application of PCT Application No. PCT/JP2021/034817 filed on Sep. 22, 2021. The entire contents of each application are hereby incorporated herein by reference.

### BACKGROUND OF THE INVENTION

### 1. Field of the Invention

The present invention relates to fluid characteristic sensors.

### 2. Description of the Related Art

For example, Japanese Unexamined Patent Application Publication No. 2009-42100 discloses a viscosity measurement method for measuring the viscosity of a liquid as one fluid characteristic. The viscosity measurement method described in Japanese Unexamined Patent Application Publication No. 2009-42100 measures the viscosity of a liquid by using a thin tube flow path and measuring a flow velocity of the liquid to be measured flowing through the thin tube flow path. Additionally, in the viscosity measurement method described in Japanese Unexamined Patent Application Publication No. 2009-42100, the flow velocity is measured by measuring a flow current generated in the thin tube flow path when the liquid to be measured flows through the thin tube flow path.

However, in Japanese Unexamined Patent Application Publication No. 2009-42100, there is still room for improvement in terms of measuring characteristics of various fluids.

### SUMMARY OF THE INVENTION

Preferred embodiments of the present invention provide fluid characteristic sensors that are each able to measure characteristics of various fluids.

A fluid characteristic sensor according to a preferred embodiment of the present invention is configured to measure a characteristic of a fluid to be measured, the fluid characteristic sensor including a pressure loss generator to generate a pressure loss when the fluid flows, a first flow path connected to the pressure loss generator and through which the fluid and a working liquid that is a polar solvent flow, a partition wall provided in the first flow path to partition the fluid and the working liquid from each other, and a potential measurer connected to the first flow path to measure a flow potential generated when the working liquid flows.

According to preferred embodiments of the present invention, it is possible to provide fluid characteristic sensors that are each able to measure characteristics of various fluids.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic configuration diagram of an example of a fluid characteristic sensor according to Preferred Embodiment 1 of the present invention.

FIG. 2 is a block diagram illustrating a main configuration of an example of the fluid characteristic sensor according to Preferred Embodiment 1 of the present invention.

FIG. 3 is a flowchart of an example of a measurement method according to Preferred Embodiment 1 of the present invention.

FIG. 4A is a schematic diagram illustrating an example of an operation of the fluid characteristic sensor according to Preferred Embodiment 1 of the present invention.

FIG. 4B is a schematic diagram illustrating an example of an operation of the fluid characteristic sensor according to Preferred Embodiment 1 of the present invention.

FIG. 4C is a schematic diagram illustrating an example of an operation of the fluid characteristic sensor according to Preferred Embodiment 1 of the present invention.

FIG. 5 is a graph illustrating an example of a change in flow potential measured with the fluid characteristic sensor according to Preferred Embodiment 1 of the present invention.

FIG. 6A is a schematic diagram illustrating an example of a manufacturing process of the fluid characteristic sensor according to Preferred Embodiment 1 of the present invention.

FIG. 6B is a schematic diagram illustrating an example of the manufacturing process of the fluid characteristic sensor according to Preferred Embodiment 1 of the present invention.

FIG. 6C is a schematic diagram illustrating an example of the manufacturing process of the fluid characteristic sensor according to Preferred Embodiment 1 of the present invention.

FIG. 6D is a schematic diagram illustrating an example of the manufacturing process of the fluid characteristic sensor according to Preferred Embodiment 1 of the present invention.

FIG. 7 is a schematic configuration diagram of a fluid characteristic sensor according to Modification 1 of Preferred Embodiment 1 of the present invention.

FIG. 8 is a schematic configuration diagram of a fluid characteristic sensor according to Modification 2 of Preferred Embodiment 1 of the present invention.

FIG. 9 is a schematic configuration diagram of a fluid characteristic sensor according to Modification 3 of Preferred Embodiment 1 of the present invention.

FIG. 10 is a graph illustrating an example of changes in flow potentials of three measurement targets measured with the fluid characteristic sensor according to Preferred Embodiment 1 of the present invention.

FIG. 11 is a graph illustrating an example of a relationship between the reciprocals of flow potential measurement values at a time t in the graph of FIG. 10 and viscosities of the measurement targets.

FIG. 12 is a schematic configuration diagram of an example of a fluid characteristic sensor according to Preferred Embodiment 2 of the present invention.

FIG. 13 is a block diagram illustrating a main configuration of an example of the fluid characteristic sensor according to Preferred Embodiment 2 of the present invention.

FIG. 14 is a flowchart of an example of a measurement method according to Preferred Embodiment 2 of the present invention.

FIG. 15A is a schematic diagram illustrating an example of an operation of the fluid characteristic sensor according to Preferred Embodiment 2 of the present invention.

FIG. 15B is a schematic diagram illustrating an example of an operation of the fluid characteristic sensor according to Preferred Embodiment 2 of the present invention.

FIG. 15C is a schematic diagram illustrating an example of an operation of the fluid characteristic sensor according to Preferred Embodiment 2 of the present invention.

FIG. 16 is a graph illustrating an example of a change in flow potential measured with the fluid characteristic sensor according to Preferred Embodiment 2 of the present invention.

FIG. 17 is a graph illustrating another example of a change in flow potential measured with the fluid characteristic sensor according to Preferred Embodiment 2 of the present invention.

FIG. 18 is a schematic diagram illustrating another example of an operation of the fluid characteristic sensor according to Preferred Embodiment 2 of the present invention.

FIG. 19 is a flowchart of an example of a measurement method according to Modification 4 of Preferred Embodiment 2 of the present invention.

FIG. 20 is a flowchart of an example of a measurement method according to Preferred Embodiment 3 of the present invention.

FIG. 21 is a graph illustrating an example of a change in flow potential measured with the fluid characteristic sensor according to Preferred Embodiment 3 of the present invention.

FIG. 22 is a table showing an example of measurement conditions and measurement results of Examples 1 to 3.

FIG. 23 is a graph illustrating an example of relationships between viscosities and shear rates in Example 1 and Example 3.

FIG. 24 is a flowchart of an example of a measurement method according to Preferred Embodiment 4 of the present invention.

FIG. 25 is a table showing an example of measurement conditions and measurement results in Example 4 and Example 5.

FIG. 26 is a graph illustrating an example of relationships between viscosities and shear rates in Example 4 and Example 5.

FIG. 27 is a flowchart of an example of a measurement method according to Preferred Embodiment 5 of the present invention.

FIG. 28 is a graph illustrating an example of a change in flow potential measured with the fluid characteristic sensor according to Preferred Embodiment 5 of the present invention.

FIG. 29 is a table showing an example of measurement conditions and measurement results of Examples 6 to 9.

FIG. 30 is a graph illustrating an example of relationships between viscosities and shear rates in Examples 6 to 9.

FIG. 31 is a schematic diagram of a fluid characteristic sensor according to Comparative Example 1.

FIG. 32 is a graph illustrating an example of a change in flow potential measured with the fluid characteristic sensor according to Comparative Example 1.

FIG. 33 is a graph illustrating an example of a change in flow potential measured with a fluid characteristic sensor according to Comparative Example 2.

FIG. 34 is a graph illustrating an example of a change in flow potential measured with a fluid characteristic sensor according to Comparative Example 3.

FIG. 35 is a schematic configuration diagram of an example of a fluid characteristic sensor according to Preferred Embodiment 6 of the present invention.

FIG. 36 is a schematic exploded diagram of the fluid characteristic sensor illustrated in FIG. 35.

FIG. 37A is a schematic diagram illustrating an example of a manufacturing process of the fluid characteristic sensor according to Preferred Embodiment 6 of the present invention.

FIG. 37B is a schematic diagram illustrating an example of the manufacturing process of the fluid characteristic sensor according to Preferred Embodiment 6 of the present invention.

FIG. 37C is a schematic diagram illustrating an example of the manufacturing process of the fluid characteristic sensor according to Preferred Embodiment 6 of the present invention.

FIG. 37D is a schematic diagram illustrating an example of the manufacturing process of the fluid characteristic sensor according to Preferred Embodiment 6 of the present invention.

FIG. 38 is a schematic configuration diagram of an example of a fluid characteristic sensor according to Preferred Embodiment 7 of the present invention.

FIG. 39 is a schematic configuration diagram of an example of a fluid characteristic sensor according to Modification 5 of Preferred Embodiment 7 of the present invention.

FIG. 40 is a schematic configuration diagram of an example of a fluid characteristic sensor according to Modification 6 of Preferred Embodiment 7 of the present invention.

FIG. 41 is a schematic diagram illustrating another example of a solid partition wall.

FIG. 42 is a schematic configuration diagram of an example of a fluid characteristic sensor according to Modification 7 of Preferred Embodiment 7 of the present invention.

FIG. 43A is a schematic diagram for describing an example of an operation of the solid partition wall.

FIG. 43B is a schematic diagram for describing an example of an operation of the solid partition wall.

FIG. 44 is a schematic configuration diagram of an example of a fluid characteristic sensor according to Modification 8 of Preferred Embodiment 7 of the present invention.

FIG. 45 is a schematic configuration diagram of an example of a fluid characteristic sensor according to Modification 9 of Preferred Embodiment 7 of the present invention.

FIG. 46 is a schematic configuration diagram of an example of a fluid characteristic sensor according to Modification 10 of Preferred Embodiment 7 of the present invention.

FIG. 47 is a schematic configuration diagram of an example of a fluid characteristic sensor according to Modification 11 of Preferred Embodiment 7 of the present invention.

FIG. 48A is a schematic diagram for describing an example of an operation of a solid partition wall in Modification 11.

FIG. 48B is a schematic diagram for describing an example of an operation of the solid partition wall in Modification 11.

FIG. 49 is a schematic diagram illustrating another example of the solid partition wall.

FIG. 50 is a schematic configuration diagram of an example of a fluid characteristic sensor according to Preferred Embodiment 8 of the present invention.

FIG. 51 is a schematic configuration diagram of an example of a fluid characteristic sensor according to Modification 12 of Preferred Embodiment 8 of the present invention.

FIG. 52 is a schematic configuration diagram of an example of a fluid characteristic sensor according to Modification 13 of Preferred Embodiment 8 of the present invention.

FIG. 53 is a schematic diagram illustrating another preferred embodiment of the present invention.

FIG. 54 is a schematic diagram illustrating another preferred embodiment of the present invention.

### DETAILED DESCRIPTION OF THE PREFERRED EMBODIMENTS

In the viscosity measurement method described in Japanese Unexamined Patent Application Publication No. 2009-42100, a flow current of a liquid to be measured is quantitatively measured, and a viscosity is calculated based on the measured flow current. In addition, in the viscosity measurement method described in Japanese Unexamined Patent Application Publication No. 2009-42100, the flow current of the liquid itself to be measured is measured.

However, in the viscosity measurement method described in Japanese Unexamined Patent Application Publication No. 2009-42100, it is difficult to measure the viscosity of a liquid in which a large flow current is not easily generated. For example, a non-polar solvent such as oil is unlikely to generate a flow current large enough to measure the viscosity. For this reason, in the viscosity measurement method described in Japanese Unexamined Patent Application Publication No. 2009-42100, it is difficult to measure a viscosity of a liquid such as a non-polar solvent, and a measurement target is substantially limited to a liquid such as a polar solvent in which a flow current is easily generated.

Accordingly, as a result of intensive studies, the inventors of preferred embodiments of the present invention have discovered a configuration in which a fluid to be measured and a working liquid are separated from each other with a partition wall, and a flow potential generated when the working liquid flows is measured, and have developed the following preferred embodiments of the present invention.

A fluid characteristic sensor according to a preferred embodiment of the present invention is configured to measure a characteristic of a fluid to be measured, the fluid characteristic sensor including a pressure loss generator to generate a pressure loss when the fluid flows, a first flow path connected to the pressure loss generator and through which the fluid and a working liquid that is a polar solvent flow, a partition wall provided in the first flow path to partition the fluid and the working liquid from each other, and a potential measurer connected to the first flow path to measure a flow potential generated when the working liquid flows.

With such a configuration, characteristics of various fluids can be measured. In addition, it is also possible to measure a liquid in which a flow potential is hardly generated.

The pressure loss generator may include a thin tube with a flow path cross-sectional area smaller than a flow path cross-sectional area of the first flow path, or a porous body including a plurality of holes.

According to such a configuration, when the liquid flows in the pressure loss generator, a large pressure loss is generated according to a characteristic of a fluid, and characteristics of various fluids can be measured.

The potential measurer may include a first electrode through which the working liquid is able to pass, a second electrode located at an interval from the first electrode and through which the working liquid is able to pass, and a second flow path provided between the first electrode and the second electrode to be filled with the working liquid, and the second flow path may include a thin tube with a flow path cross-sectional area smaller than a flow path cross-sectional area of the first flow path, or a porous body including a plurality of holes.

With this configuration, it is possible to measure a flow potential generated when the working liquid flows.

The working liquid may have at least one of a boiling point higher than a boiling point of water and a melting point lower than a melting point of water.

With such a configuration, an environmental resistance can be improved.

The partition wall may include a gas, and the first flow path extends in a gravity direction, and in the first flow path, an interface between the working liquid and the partition wall may be above an interface between the fluid and the partition wall.

According to such a configuration, a surface tension generated between the working liquid and an inner wall of the first flow path reduces or prevents natural flow down of the working liquid in the gravity direction, and the interface between the working liquid and the partition wall can be maintained.

An inner wall of the first flow path may have hydrophobicity.

With such a configuration, the surface tension generated between the working liquid and the inner wall of the first flow path can be increased, and the interface between the working liquid and the partition wall can be easily maintained.

A pump to feed the working liquid and connected to the potential measurer may be provided.

With such a configuration, the liquid is caused to flow through the partition wall by feeding the working liquid.

The pump may be an electroosmotic flow pump, and may include a third electrode through which the working liquid is capable of passing, a fourth electrode located at an interval from the third electrode and through which the working liquid is capable of passing, and a third flow path between the third electrode and the fourth electrode to be filled with the working liquid, and the third flow path may include a porous body including a plurality of holes.

With such a configuration, it is possible to reduce a size of the pump and improve the degree of freedom in installation in an apparatus.

The fluid characteristic sensor may further include a pump controller configured or programmed to control a liquid feeding direction and a liquid feeding pressure of the pump, and the liquid feeding direction is a first direction from the pressure loss generator toward the pump and a second direction opposite to the first direction, the second direction extending from the pump toward the pressure loss generator.

With such a configuration, the liquid can be sucked and discharged by changing the liquid feeding direction of the working liquid. This enables continuous operation.

The pump controller may be configured or programmed to control the liquid feeding direction of the pump based on a measurement value of the flow potential measured by the potential measurer.

With such a configuration, the liquid feeding direction of the pump can be changed at an appropriate time.

The pump controller may be configured or programmed to switch the liquid feeding direction to the second direction after the measurement value of the flow potential converges when the liquid feeding direction is the first direction, and stop the pump when the liquid feeding direction is the second direction and an absolute value of a change amount in flow potential per unit time increases beyond a threshold value.

With such a configuration, pump control can be performed at a more appropriate timing.

The partition wall may have a volume larger than a flow path volume of the pressure loss generator, and the pump controller may be configured or programmed to stop the pump when the liquid feeding direction is the second direction and an absolute value of a change amount in measurement value of a flow potential per unit time decreases beyond a predetermined threshold value.

With such a configuration, it is possible to reduce or prevent flow of the working liquid to the outside of the fluid characteristic sensor.

The pump controller may be configured or programmed to change the liquid feeding pressure in a stepwise manner.

With such a configuration, it is possible to measure characteristics of more various fluids.

The fluid characteristic sensor may further include a calculator to calculate a characteristic of the fluid based on the flow potential measured by the potential measurer.

With such a configuration, it is possible to calculate the characteristic of the fluid by the fluid characteristic sensor alone.

The fluid characteristic sensor may further include a calculator to calculate a characteristic of the fluid based on the flow potential measured by the potential measurer, the calculator may calculate a first viscosity of the fluid based on a measurement value of the flow potential when the liquid feeding direction is the first direction, and may calculate a second viscosity of the fluid based on a measurement value of the flow potential when the liquid feeding direction is the second direction, and may determine the characteristic of the fluid based on the first viscosity and the second viscosity.

With such a configuration, it is possible to calculate the characteristic of the fluid by the fluid characteristic sensor alone. In addition, the characteristic of the fluid can be calculated based on the information about the viscosities.

The pump controller may set the liquid feeding pressure of the pump when the liquid feeding direction is the first direction to a first pressure, and may set the liquid feeding pressure of the pump when the liquid feeding direction is the second direction to a second pressure different from the first pressure.

With such a configuration, it is possible to measure characteristics of more various fluids.

The calculator may calculate a flow velocity of the working liquid based on the flow potential measured by the potential measurer, and may calculate a viscosity of the fluid based on the flow velocity of the working liquid.

With such a configuration, the flow velocity can be calculated from the flow potential generated when the working liquid flows, and the viscosity of the fluid can be calculated.

The fluid characteristic sensor may further include a working liquid flow path that includes an open end opened to an atmosphere side and through which the working liquid flows, and a liquid surface of the working liquid positioned at a side of the open end of the working liquid flow path may be covered with a non-polar solvent.

With such a configuration, it is possible to reduce or prevent mixture of a foreign matter into the working liquid and to improve environmental resistance.

A boiling point of the non-polar solvent may be higher than a boiling point of the working liquid.

With such a configuration, it is possible to reduce or prevent a decrease in liquid amount due to gasification of the working liquid.

The non-polar solvent may be a non-volatile solvent.

With such a configuration, it is possible to reduce or prevent a decrease in liquid amount due to volatilization of the working liquid.

The fluid characteristic sensor may further include an attachment portion including an inflow/outflow port through which the fluid flows in and out and the pressure loss generator, and a main body including at least a portion of the first flow path, the main body being detachably attached with the attachment portion.

With such a configuration, usability for a user is improved.

The partition wall may be solid.

With such a configuration, the fluid and the working liquid can be easily separated from each other.

The partition wall may include a partition wall main body being elastically deformable and having a recessed shape, and a flange protruding outward from an outer wall of the partition wall main body.

With such a configuration, the fluid characteristic sensor can be miniaturized.

The partition wall may include a plurality of the partition walls.

With such a configuration, it is possible to further reduce or prevent leakage of the working liquid.

A fluid characteristic sensor according to a preferred embodiment of the present invention is configured to measure a characteristic of a fluid to be measured, the fluid characteristic sensor including a first flow path through which the fluid and a working liquid that is a polar solvent flow, the first flow path including one end and another end, a partition wall located in the first flow path, the partition wall being configured to partition the fluid and the working liquid from each other, a pressure loss generator connected to a side of the one end of the first flow path and including a flow path cross-sectional area smaller than a flow path cross-sectional area of the first flow path, and a potential measurer connected to a side of the other end of the first flow path to measure a flow potential generated when the working liquid flows.

With such a configuration, characteristics of various fluids can be measured. In addition, it is also possible to measure a liquid in which a flow potential is hardly generated.

Hereinafter, preferred embodiments of the present invention will be described with reference to the accompanying drawings. The following description is merely exemplary in nature, and is not intended to limit the scope of the present invention, applications thereof, or usages thereof. Further, the drawings are schematically illustrated, and ratios of dimensions and the like may not necessarily coincide with actual ones.

### Preferred Embodiment 1

### Overall Configuration

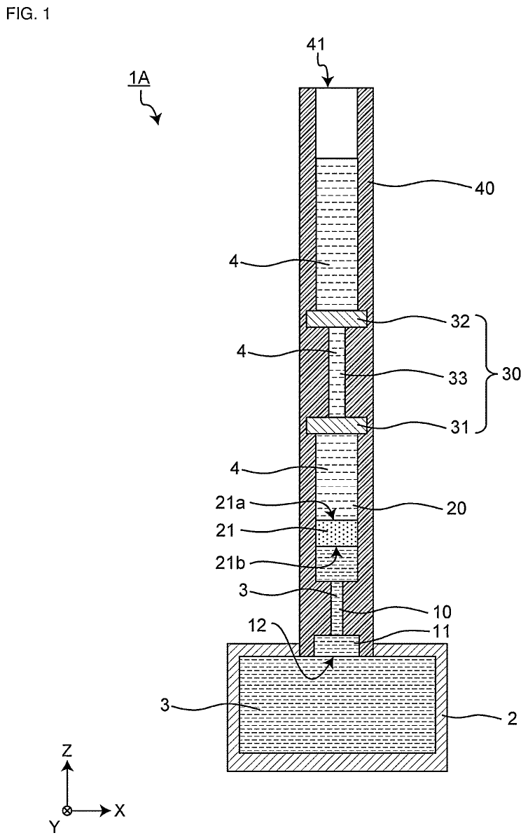

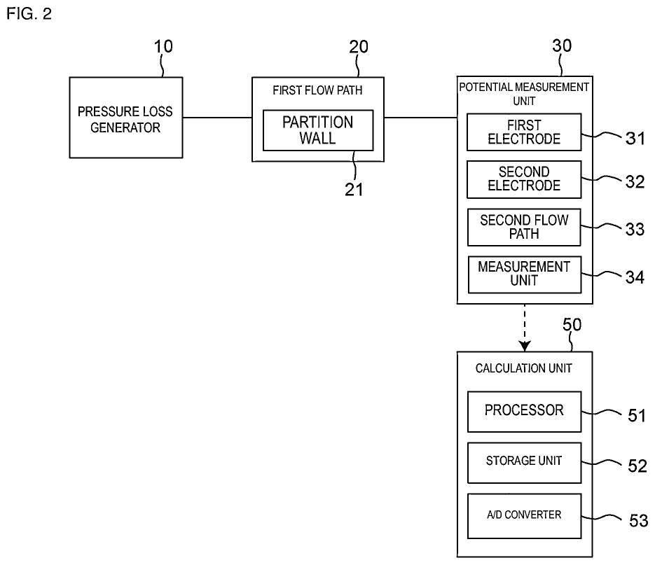

FIG. 1 is a schematic configuration diagram of an example of a fluid characteristic sensor 1A according to Preferred Embodiment 1 of the present invention. FIG. 2 is a block diagram illustrating a main configuration of an example of the fluid characteristic sensor 1A according to Preferred Embodiment 1 of the present invention. X, Y, and Z directions in the drawings indicate a width direction, a depth direction, and a height direction of the fluid characteristic sensor 1A, respectively.

A fluid characteristic sensor is a sensor that measures a characteristic of a fluid. Examples of the fluid include a liquid, a solid-liquid mixed fluid (sol), a liquid-liquid mixed fluid, and a gas-liquid mixed fluid. The characteristics of the fluid include, for example, at least one of viscosity and rheology characteristics. In Preferred Embodiment 1, a fluid characteristic sensor 1A that measures a viscosity of a liquid 3 stored in a container 2 will be described as an example.

As illustrated in FIG. 1 and FIG. 2, the fluid characteristic sensor 1A includes a pressure loss generator 10, a first flow path 20, a partition wall 21, and a potential measurer 30. In Preferred Embodiment 1, the pressure loss generator 10, the first flow path 20, and the potential measurer 30 are sequentially connected along the height direction (Z direction) of the fluid characteristic sensor 1A. To be specific, in the height direction (Z direction) of the fluid characteristic sensor 1A, the pressure loss generator 10, the first flow path 20, and the potential measurer 30 are disposed in this order from the bottom to the top.

In Preferred Embodiment 1, an example will be described in which the fluid characteristic sensor 1A includes a working liquid flow path 40 including an open end 41 opened to an atmosphere side above the potential measurer 30. In the fluid characteristic sensor 1A, the working liquid flow path 40 is not necessarily provided.

### Pressure Loss Generator

In the pressure loss generator 10, a pressure loss is generated when the liquid 3 to be measured flows. The pressure loss generator 10 includes a flow path through which the liquid 3 is capable of flowing and that generates a pressure loss. In Preferred Embodiment 1, the pressure loss generator 10 is a thin tube. Specifically, the pressure loss generator 10 is a thin tube with a flow path cross-sectional area smaller than a flow path cross-sectional area of the first flow path 20. The “flow path cross-sectional area” is an area of the flow path when a cross section obtained by taking the pressure loss generator 10 or the first flow path 20 along an XY cross section is viewed from the Z direction. For example, the flow path cross-sectional area of the thin tube defining the pressure loss generator 10 is, for example, equal to or less than about 1/10 times the flow path cross-sectional area of the first flow path 20. The flow path cross-sectional area of the thin tube defining the pressure loss generator 10 is preferably, for example, equal to or larger than about 100 μm². For example, the thin tube has a cylindrical or substantially cylindrical shape.

The thin tube defining and functioning as the pressure loss generator 10 includes one end and another end. A fluid flow path 11 connected to the container 2 that stores the liquid 3 is provided at the one end of the thin tube. The first flow path 20 is provided at the other end of the thin tube. The fluid flow path 11 includes an inflow/outflow port 12 through which the liquid 3 flows in and out. The fluid flow path 11 has a flow path cross-sectional area larger than a flow path cross-sectional area of the thin tube. For example, the fluid flow path 11 may have the same or substantially the same flow path cross-sectional area as a flow path cross-sectional area of the first flow path 20. The expression of “substantially the same” means that an error within about 10% is included. In the height direction (Z direction) of the fluid characteristic sensor 1A, a length of the fluid flow path 11 is shorter than a length of the pressure loss generator 10.

The liquid 3 to be measured may be, for example, a polar solvent or a non-polar solvent.

### First Flow Path

The first flow path 20 is connected to the pressure loss generator 10, and the liquid 3 and a working liquid 4 flow through the first flow path 20. The working liquid 4 is a liquid in which a flow potential is generated due to flowing. The working liquid 4 is a polar solvent. The working liquid 4 has at least one of a boiling point higher than the boiling point of water and a melting point lower than the melting point of water. For example, a boiling point higher than the boiling point of water means a temperature higher than 100° C. under atmospheric pressure. A melting point lower than the melting point of water means a temperature lower than 0° C. under atmospheric pressure. For example, the working liquid 4 may be any one of water, ethylene glycol, propylene glycol, diethyl glycol, tetraethylene glycol, glycerin, dimethyl sulfoxide, dimethyl formamide, an antifreeze solution, a heat medium, an electrolyte aqueous solution, and a buffer solution. The antifreeze solution means a liquid prepared so as not to be frozen in winter (cold region), and is standardized in Japanese Industrial Standards (JIS) as JIS K 2234 antifreeze solution. As the antifreeze solution, for example, a water-ethylene glycol mixed solution or the like is used. As the heat medium, for example, hydrofluorocarbon or the like is used. As the electrolyte aqueous solution, for example, a NaCl aqueous solution, a KCl aqueous solution, or the like is used. As the buffer solution, for example, a phosphate buffer solution, a borate buffer solution, a Good's buffer, a Tris buffer solution, or the like is used. The working liquid 4 is preferably a liquid that can operate under an environment at a high temperature being equal to or higher than 100° C. and/or an environment at a low temperature being equal to or lower than 0° C. For example, when ethylene glycol is used as the working liquid 4, the melting point is lower than 0° C., which allows driving below the freezing point. In addition, when tetraethylene glycol is used, the boiling point is higher than 100° C., which allows driving at a high temperature.

The liquid 3 and the working liquid 4 that is a polar solvent flow through the first flow path 20, and the first flow path 20 includes one end and another end. In Preferred Embodiment 1, the first flow path 20 is a pipe including one end and the other end. The one end of the first flow path 20 is connected to the other end of the thin tube that is the pressure loss generator 10. The other end of the first flow path 20 is connected to the potential measurer 30. For example, the first flow path 20 has a cylindrical shape.

### Partition Wall

The partition wall 21 is disposed in the first flow path 20 and partitions the liquid 3 and the working liquid 4 from each other. The partition wall 21 is movable in the height direction (Z direction) of the fluid characteristic sensor 1A according to the flow of the liquid 3 and the working liquid 4. In Preferred Embodiment 1, the partition wall 21 is, for example, a gas. The partition wall 21 is, for example, an inert gas capable of preventing an undesirable chemical reaction from occurring due to contact with the measurement target or the working liquid 4. For example, the partition wall 21 is made of air or argon. Hereinafter, the “partition wall 21” may be referred to as a “movable partition wall 21”.

The height direction (Z direction) of the fluid characteristic sensor 1A is along a gravity direction. The first flow path 20 extends in the gravity direction. Thus, in the first flow path 20, the liquid 3, the movable partition wall 21, and the working liquid 4 are positioned in this order from the bottom to the top. In other words, in the first flow path 20, an interface 21a between the working liquid 4 and the movable partition wall 21 is provided at a position above than an interface 21b between the liquid 3 and the movable partition wall 21. In the first flow path 20, a surface tension acts between the working liquid 4 and an inner wall 20a of the first flow path 20, so that the working liquid 4 is less likely to naturally fall in the gravity direction. As a result, a shape of the movable partition wall 21 that is a gas is easily maintained, and the interface 21a between the movable partition wall 21 and the working liquid 4 can be maintained.

In addition, the inner wall 20a of the first flow path 20 may have hydrophobicity. For example, the first flow path 20 is made of a hydrophobic material. Examples of the hydrophobic material include ABS, nylon, polyacetal, fluorine resin, polytetrafluoroethylene (PTFE), and polyetheretherketone (PEEK). Alternatively, hydrophobic coating is applied to the inner wall 20a of the first flow path 20. As a result, the surface tension generated on the inner wall 20a of the first flow path 20 can be increased, and the working liquid 4 is further less likely to naturally fall in the gravity direction.

### Potential Measurer

The potential measurer 30 is connected to the first flow path 20 and measures the flow potential of the working liquid 4. The flow potential means a potential difference generated on a solid surface when a liquid being in contact with the solid surface flows.

The potential measurer 30 includes a first electrode 31, a second electrode 32, and a second flow path 33.

The first electrode 31 and the second electrode 32 are made of a material through which the working liquid 4 can pass. The first electrode 31 and the second electrode 32 are made of, for example, a porous conductive material. As the porous conductive material, for example, a metal material such as Pt, Cu, Ag, Au, Ni, or stainless steel, or a carbon electrode can be used. The porous conductive material may be any material that has conductivity and that can ensure water permeability. For example, the porous conductive material may be a conductive rubber, an oxide conductor, or the like. In Preferred Embodiment 1, each of the first electrode 31 and the second electrode 32 is made of a metal mesh having a flat plate shape and including two main surfaces facing each other.

The first electrode 31 and the second electrode 32 are spaced apart from each other. Specifically, the first electrode 31 and the second electrode 32 face each other with an interval therebetween in the flow direction (Z direction) of the working liquid 4. In addition, the main surfaces of the first electrode 31 and the second electrode 32 are disposed in a direction intersecting the flow direction (Z direction) of the liquid.

The second flow path 33 is disposed between the first electrode 31 and the second electrode 32, and is filled with the working liquid 4. In Preferred Embodiment 1, the second flow path 33 is a thin tube through which the working liquid 4 flows. Specifically, the second flow path 33 is a thin tube with a flow path cross-sectional area smaller than the flow path cross-sectional area of the first flow path 20. For example, the flow path cross-sectional area of the thin tube forming the second flow path 33 is less than about one time the flow path cross-sectional area of the first flow path 20. The flow path cross-sectional area of the thin tube defining the second flow path 33 is preferably, for example equal to or larger than about 100 μm². For example, the thin tube has a cylindrical or substantially cylindrical shape.

The thin tube defining the second flow path 33 includes one end and another end. The first electrode 31 is disposed at the one end of the thin tube. The second electrode 32 is disposed at the other end of the thin tube.

In Preferred Embodiment 1, the potential measurer 30 includes a measurer 34 connected to the first electrode 31 and the second electrode 32. The measurer 34 measures a voltage between the first electrode 31 and the second electrode 32. For example, the measurer 34 is an electrometer. In the fluid characteristic sensor 1A, the measurer 34 is not necessarily provided. For example, the measurer 34 may be included in a device different from the fluid characteristic sensor 1A.

The working liquid flow path 40 is connected to the potential measurer 30. The working liquid flow path 40 is a pipe including the open end 41 at an atmosphere side. For example, the working liquid flow path 40 has a cylindrical or substantially cylindrical shape. The working liquid 4 is provided in the working liquid flow path 40. The working liquid 4 flows in the working liquid flow path 40. For example, a pump, a syringe, or the like is attached to the working liquid flow path 40. Thus, the liquid 3 and the working liquid 4 in the fluid characteristic sensor 1A can be caused to flow.

In Preferred Embodiment 1, as illustrated in FIG. 2, the fluid characteristic sensor 1A includes a calculator 50. The calculator 50 calculates a characteristic of the liquid 3 based on the flow potential measured by the potential measurer 30. Specifically, the calculator 50 calculates a viscosity of the liquid 3 based on the flow potential measured by the potential measurer 30.

The calculator 50 includes a processor 51, a storage 52, and an A/D converter 53.

The processor 51 is, for example, a central processing unit (CPU), a microprocessor, or a circuit capable of executing instructions in a computer. For example, the processor 51 can execute the instructions or a program stored in the storage 52.

The storage 52 is, for example, a computer recording medium that stores the instructions or the program to be executed by the processor 51. The storage 52 may be, for example, a RAM, a ROM, an EEPROM, a flash memory, or another memory technique, a CD-ROM, a DVD, or another optical disk storage, or a magnetic cassette, a magnetic tape, a magnetic disk storage, or another magnetic storage device.

The A/D converter 53 converts an analog signal into a digital signal. In Preferred Embodiment 1, the A/D converter 53 converts the flow potential measured by the potential measurer 30 into a digital signal.

In the fluid characteristic sensor 1A, the calculator 50 is not necessarily provided. For example, the calculator 50 may be included in a device different from the fluid characteristic sensor 1A.

### Example of Calculation of Viscosity Based on Flow Potential

The viscosity of the liquid 3 to be measured can be calculated from, for example, the Hagen-Poiseuille equation representing a relationship between a pressure loss and a flow rate. The Hagen-Poiseuille equation is shown below.

$$Q = \frac{π {r}^{4}}{8 η L} \cdot Δ P \qquad Math . 1$$

Here, Q is a flow rate, ΔP is a pressure difference (pressure loss), η is a viscosity of the liquid 3, L is a length of the thin tube, and r is a radius of the thin tube. ΔP, L, and r are determined by dimensions of the thin tube that is the pressure loss generator 10.

As can be seen from the Hagen-Poiseuille equation, the flow rate Q is determined according to the viscosity η of the liquid 3. In other words, the viscosity η of the liquid 3 can be calculated from the Hagen-Poiseuille equation by measuring the flow rate Q.

The flow rate Q of the liquid 3 is equal or substantially equal to the flow rate of the working liquid 4 flowing along with the flow of the liquid 3. The flow rate of the working liquid 4 can be calculated from a flow velocity of the working liquid 4, and the flow velocity of the working liquid 4 can be calculated from the flow potential. The flow potential is proportional to the flow velocity (flow rate) of the flowing working liquid 4. The fluid characteristic sensor 1A measures a flow potential generated along with the flow of the working liquid 4 by using the potential measurer 30. In addition, the fluid characteristic sensor 1A calculates the flow velocity (flow rate) of the working liquid 4 based on the measured flow potential by the calculator 50. Since the flow velocity (flow rate) of the working liquid 4 is equal or substantially equal to the flow velocity (flow rate) of the liquid 3 to be measured, the flow rate Q of the liquid 3 can be obtained from the flow velocity (flow rate) of the working liquid 4.

### Operation

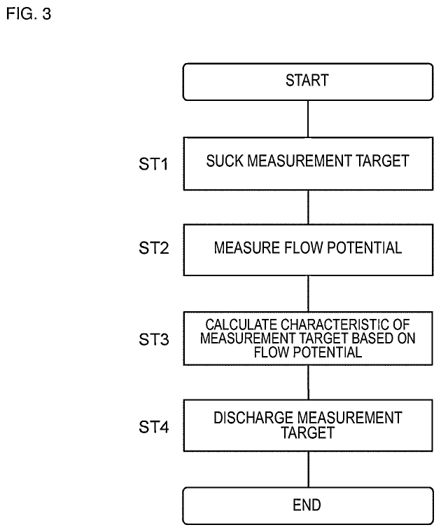

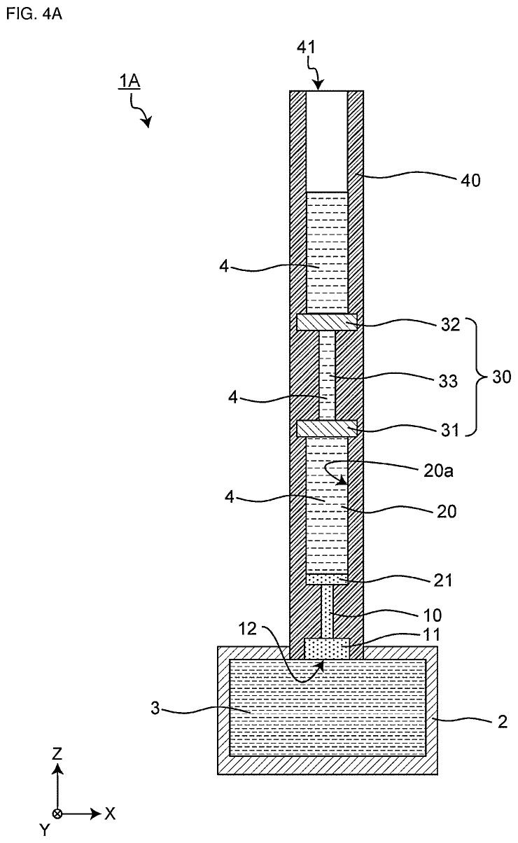

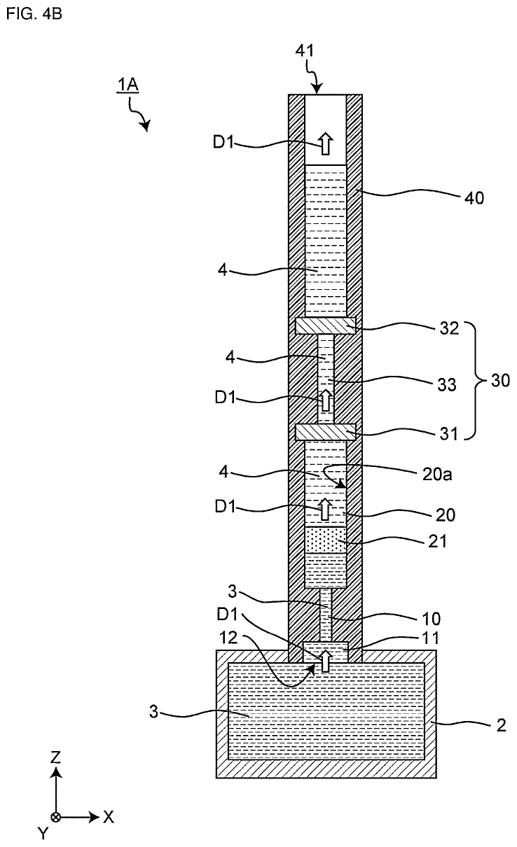

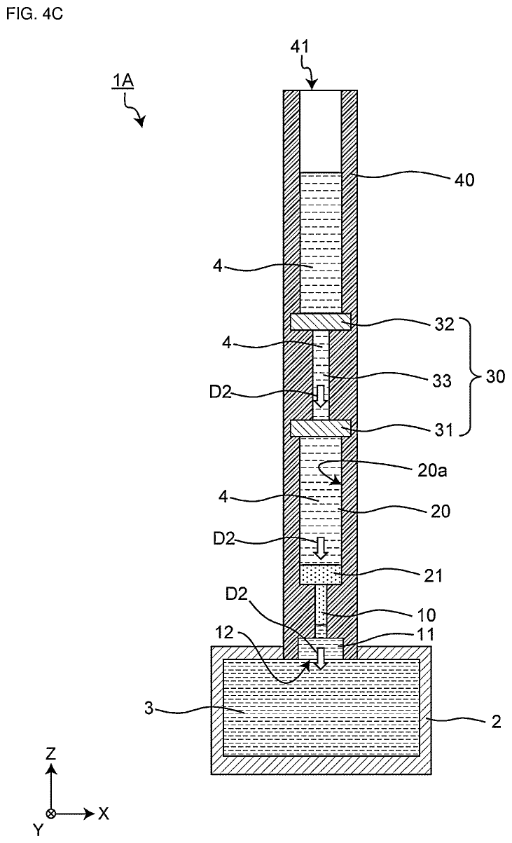

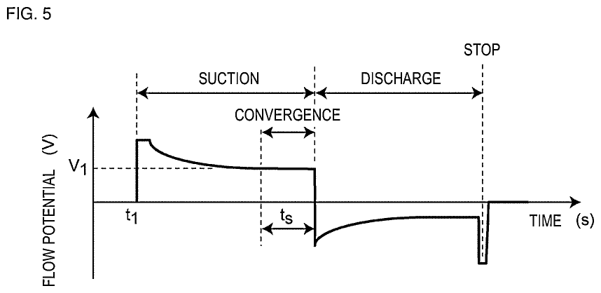

An operation of the fluid characteristic sensor 1A, that is, an example of a measurement method will be described with reference to FIG. 3 to FIG. 5. FIG. 3 is a flowchart of an example of the measurement method according to Preferred Embodiment 1 of the present invention. FIGS. 4A to 4C are schematic views illustrating an example of the operation of the fluid characteristic sensor 1A according to Preferred Embodiment 1 of the present invention. FIG. 5 is a graph illustrating an example of a change in flow potential measured by the fluid characteristic sensor 1A according to Preferred Embodiment 1 of the present invention. The operation will be described with respect to an example in which a viscosity is measured as a characteristic of the liquid 3 to be measured.

As illustrated in FIG. 3, in step ST1, the liquid 3 to be measured is sucked. To be more specific, as illustrated in FIG. 4A, the inflow/outflow port 12 of the fluid flow path 11 of the fluid characteristic sensor 1A is provided for the liquid 3 stored in the container 2. As illustrated in FIG. 4B, the liquid 3 is sucked in a first direction D1 in a state where the inflow/outflow port 12 is disposed for the liquid 3 stored in the container 2. The first direction D1 is a direction in which the liquid 3 is sucked. In Preferred Embodiment 1, the first direction D1 is a direction from the pressure loss generator 10 toward the potential measurer 30. For example, the working liquid 4 is sucked by a pump or the like disposed in the working liquid flow path 40 of the fluid characteristic sensor 1A to suck the liquid 3 in the first direction D1. Thus, the liquid 3 stored in the container 2 flows into the pressure loss generator 10 from the inflow/outflow port 12 through the fluid flow path 11. The liquid 3 that has flowed into the pressure loss generator 10 flows into the first flow path 20 while a pressure loss is being generated. The movable partition wall 21 between the liquid 3 and the working liquid 4 is disposed in the first flow path 20. When the liquid 3 flows into the first flow path 20, the working liquid 4 flows in the first direction D1 together with the movable partition wall 21. At this time, the flow rate of the flowing working liquid 4 is equal or substantially equal to the flow rate of the liquid 3 flowing into the first flow path 20. Here, the expression of “substantially equal” means, for example, that an error of several % caused by deformation of the flow path wall surface in the fluid characteristic sensor 1A and expansion and contradiction of the movable partition wall is included.

Thus, in the potential measurer 30, the working liquid 4 flows at the same or substantially the same flow rate as that of the liquid 3, that is, at the same or substantially the same flow velocity as that of the liquid 3. To be specific, in the second flow path 33 of the potential measurer 30, the working liquid 4 flows in the first direction D1 at the same or substantially the same flow rate as that of the liquid 3, that is, at the same or substantially the same flow velocity as that of the liquid 3.

Returning to FIG. 3, in step ST2, the flow potential of the working liquid 4 is measured by the potential measurer 30. Specifically, in the potential measurer 30, the measurer 34 measures the flow potential generated by the working liquid 4 flowing through the second flow path 33 disposed between the first electrode 31 and the second electrode 32.

In Step ST3, the characteristic of the liquid 3 to be measured is calculated by the calculator 50 based on the measured flow potential. Specifically, the calculator 50 calculates the viscosity of the liquid 3 based on the flow potential. As described above, the viscosity of the liquid 3 is calculated based on the flow potential by using the Hagen-Poiseuille equation.

As illustrated in FIG. 5, the flow potential increases with the start of suction at a time t₁, and decreases and converges with the lapse of time. The calculator 50 calculates the viscosity of the liquid 3 based on a measurement value when the flow potential has converged, that is, a convergence value V₁ of the flow potential. In Preferred Embodiment 1, the convergence of the flow potential is determined based on a threshold value of a change amount in the flow potential per unit time tₛ. For example, when the change amount in the flow potential for about 10 seconds is within about ±0.02 V, the calculator 50 may determine that the flow potential has converged. The unit time tₛ is not limited to about 10 seconds, and may be set to any value. Additionally, the threshold value of the change amount in the flow potential is not limited to about ±0.02 V, and may be set to any value.

Referring back to FIG. 3, in step ST4, the liquid 3 to be measured is discharged. To be specific, as illustrated in FIG. 4C, the liquid 3 is discharged in a second direction D2 in a state where the inflow/outflow port 12 is provided for the liquid 3 stored in the container 2. The second direction D2 is a direction in which the liquid 3 is discharged. In Preferred Embodiment 1, the second direction D2 is a direction opposite to the first direction D1, and is a direction from the potential measurer 30 toward the pressure loss generator 10. For example, the liquid 3 is discharged in the second direction D2 by discharging the working liquid 4 with a pump or the like disposed in the working liquid flow path 40 of the fluid characteristic sensor 1A. Thus, the liquid 3 in the first flow path 20 is pushed by the working liquid 4 with the movable partition wall 21 interposed therebetween, and passes through the pressure loss generator 10 and the fluid flow path 11 to be discharged to the container 2.

As described above, in the measurement method using the fluid characteristic sensor 1A, the viscosity can be measured as the characteristic of the liquid 3 by performing steps ST1 to ST4.

### Manufacturing Method

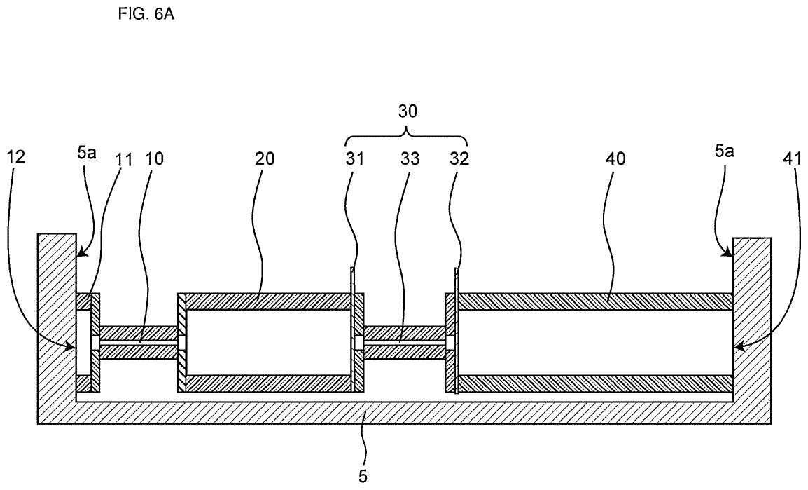

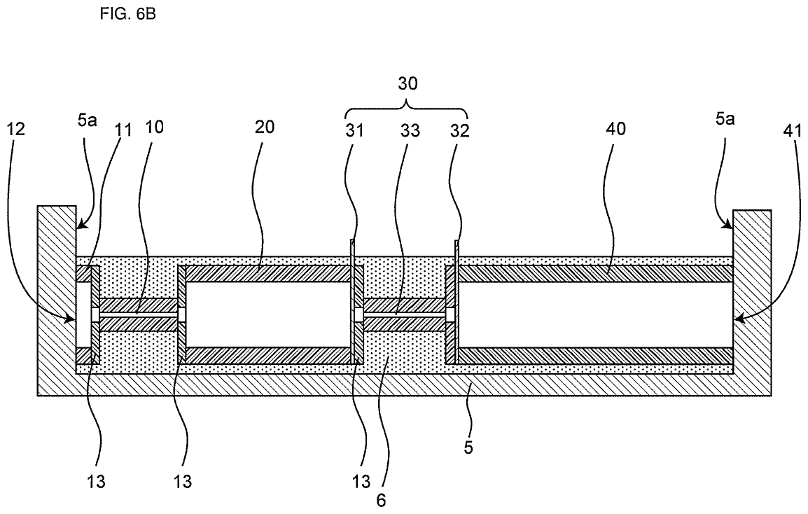

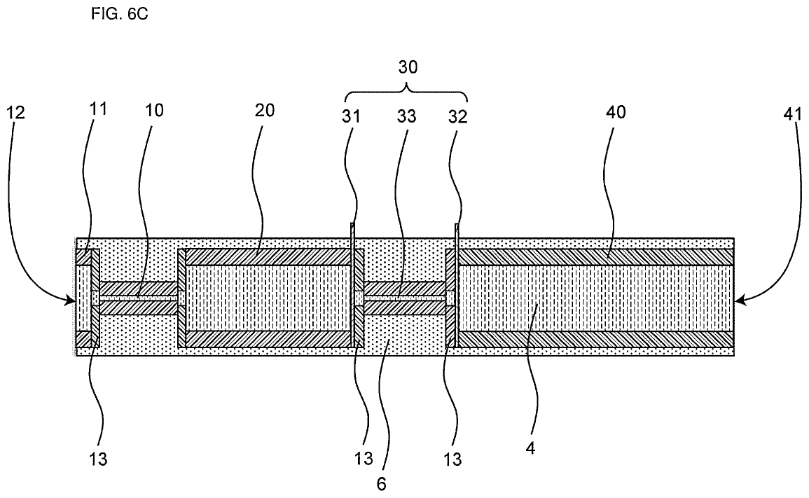

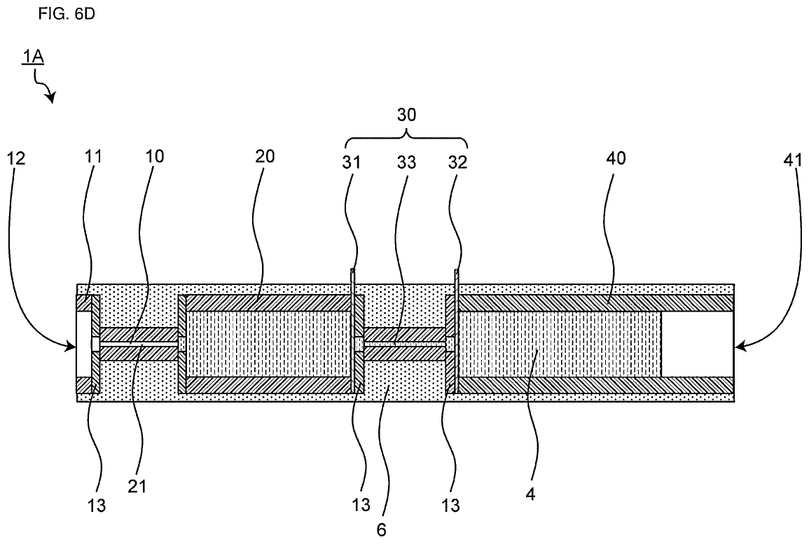

A non-limiting example of a manufacturing method of the fluid characteristic sensor 1A will be described with reference to FIGS. 6A to 6D. FIGS. 6A to 6D are schematic diagrams illustrating an example of a manufacturing process of the fluid characteristic sensor 1A according to Preferred Embodiment 1 of the present invention. Note that although an example in which resin plates 13 are provided at both ends of the pressure loss generator 10 and at both ends of the second flow path 33 is illustrated in FIGS. 6A to 6D, preferred embodiments of the present invention are not limited thereto. The resin plates 13 are not necessarily provided.

As illustrated in FIG. 6A, the elements of the fluid characteristic sensor 1A are disposed in a mold 5. The mold 5 has a recessed shape. Specifically, the fluid flow path 11, the pressure loss generator 10, the first flow path 20, the potential measurer 30, the working liquid flow path 40, and the resin plates 13 are disposed in the mold 5 in a state of being connected to each other. At this time, the inflow/outflow port 12 of the fluid flow path 11 and the open end 41 of the working liquid flow path 40 are pressed so as to close an inner wall 5a of the mold 5. That is, the inflow/outflow port 12 and the open end 41 are sealed by the inner wall 5a of the mold 5. When the elements of the fluid characteristic sensor 1A are disposed in the mold 5, these elements may be bonded by using, for example, an adhesive or the like.

As illustrated in FIG. 6B, a molten sealing material 6 is introduced into the mold 5 and cured. The sealing material 6 is, for example, a resin material. Examples of the resin material include polydimethylsiloxane (PDMS) and epoxy resin. At this time, a portion of each of the first electrode 31 and the second electrode 32 in the potential measurer 30 is exposed from the sealing material 6. The exposed portions of the first electrode 31 and the second electrode 32 function as terminals connected to the measurer 34.

As illustrated in FIG. 6C, after the sealing material 6 is cured, the mold 5 is removed. After the mold 5 is removed, a nozzle is inserted into the open end 41 of the working liquid flow path 40 to introduce the working liquid 4. Next, a syringe is attached to the open end 41, the working liquid 4 is fed toward the inflow/outflow port 12 by the syringe, and the fluid flow path 11, the pressure loss generator 10, the first flow path 20, the potential measurer 30, and the working liquid flow path 40 are filled with the working liquid 4.

As illustrated in FIG. 6D, the working liquid 4 in the fluid flow path 11 and the pressure loss generator 10 is removed. For example, the syringe is attached to the inflow/outflow port 12 to suck the working liquid 4 in the fluid flow path 11 and the pressure loss generator 10. This forms the movable partition wall 21 of a gas. Next, the nozzle is inserted into the working liquid flow path 40 from the open end 41 to suck a certain amount of the working liquid 4. The certain amount is, for example, about 0.1 ml.

In this way, the fluid characteristic sensor 1A can be manufactured.

### Example of Shape, Dimensions, and Material of Fluid Characteristic Sensor

An example of dimensions of the fluid characteristic sensor 1A will be described. The fluid flow path 11 has a cylindrical or substantially cylindrical shape with an inside diameter of about 4 mm, an outside diameter of about 6 mm, and a length of about 2 mm. The pressure loss generator 10 is a thin tube having a cylindrical or substantially cylindrical shape with an inside diameter of about 0.5 mm, an outside diameter of about 2 mm, and a length of about 10 mm. The first flow path 20 is a pipe having a cylindrical or substantially cylindrical shape with an inside diameter about 4 mm, an outside diameter about 6 mm, and a length about 20 mm. Each of the first electrode 31 and second electrode 32 of the potential measurer 30 is a metal mesh having a disc shape with a diameter of about 6 mm and a thickness of about 0.1 mm. The second flow path 33 of the potential measurer 30 is a thin tube having a cylindrical or substantially cylindrical shape with an inside diameter of about 0.5 mm, an outside diameter of about 2 mm, and a length of about 10 mm. The working liquid flow path 40 is a pipe having a cylindrical or substantially cylindrical shape with an inside diameter of about 4 mm, an outside diameter of about 6 mm, and a length of about 20 mm. The resin plate 13 has a disk shape with a hole diameter of about 1.5 mm, a diameter of about 6 mm, and a thickness of about 1 mm.

The fluid flow path 11, the pressure loss generator 10, the first flow path 20, the second flow path 33 of the potential measurer 30, the working liquid flow path 40, and the resin plates 13 can be made of, for example, ABS, nylon, polyacetal, fluororesin, PTFE, or the like. Alternatively, these elements may be made of a metal material such as SUS, for example. However, when a conductive material is used, it is necessary to ensure insulation between electrodes. In addition, by making the first flow path 20 with a hydrophobic material, a large surface tension acts on the inner wall of the flow path, and the movable partition wall 21 defined by a gas is easily maintained.

The first electrode 31 and the second electrode 32 may be made of a metal material such as, for example, Pt, Cu, Ag, Au, Ni, or stainless steel.

The above-described dimensions are merely an example, and the present invention is not limited thereto. For example, the inside diameter of the thin tube defining and functioning as the pressure loss generator 10 is preferably equal to or more than about 0.01 mm and equal to or less than about 10 mm. More preferably, the inside diameter of the thin tube is, for example, equal to or more than about 0.1 mm and equal to or less than about 1 mm. The inside diameter of the thin tube defining and functioning as the pressure loss generator 10 may be changed according to a viscosity range to be measured.

The inside diameters of the fluid flow path 11, the first flow path 20, and the working liquid flow path 40 are preferably, for example, about four times or more the inside diameters of the pressure loss generator 10 and the second flow path 33. The pressure loss is inversely proportional to the fourth power of the inside diameter of the pressure loss generator 10. For this reason, by making the inside diameters of the fluid flow path 11, the first flow path 20, and the working liquid flow path 40 about four times or more the inside diameters of the pressure loss generator 10 and the second flow path 33, the pressure loss can be reduced or prevented to be equal to or less than about 2% of that of the pressure loss generator 10.

### Advantageous Effects

According to the fluid characteristic sensor 1A of Preferred Embodiment 1, the following advantageous effects can be obtained.

The fluid characteristic sensor 1A is a fluid characteristic sensor that measures a characteristic of the liquid 3 to be measured, and includes the pressure loss generator 10, the first flow path 20, the movable partition wall 21, and the potential measurer 30. The flow of the liquid 3 in the pressure loss generator 10 causes a pressure loss. The first flow path 20 is connected to the pressure loss generator 10, and the liquid 3 and the working liquid 4 that is a polar solvent flow through the first flow path 20. The movable partition wall 21 is movably provided in the first flow path 20, and partitions the liquid 3 and the working liquid 4. The liquid 3 and the working liquid 4 flow with the movable partition wall interposed therebetween at equal or substantially equal flow rates to each other. The potential measurer 30 is connected to the first flow path 20, and measures a flow potential generated when the working liquid 4 flows in the potential measurer 30.

With such a configuration, characteristics of various fluids can be measured. In the fluid characteristic sensor 1A, since a large pressure loss is generated in the pressure loss generator 10, a flow rate (flow velocity) is determined according to a viscosity of the liquid 3. In the first flow path 20, the liquid 3 and the working liquid 4 are in a state of being partitioned by the movable partition wall 21, and the liquid 3 and the working liquid 4 flow at equal or substantially equal flow rates (flow velocity) to each other. The flow rate of the working liquid 4 can be calculated from the flow potential. Since the working liquid 4 is a polar solvent, a flow potential having a measurable level is generated with the flow of the working liquid 4. Additionally, since the working liquid 4 is a known liquid, the correlation between the flow rate and the flow potential is also known. Thus, the flow rate of the working liquid 4 is calculated based on the measured flow potential. Since the flow rates of the liquid 3 and the working liquid 4 are equal or substantially equal to each other, the flow rate Q of the liquid 3 can be obtained based on the flow rate of the working liquid 4. At this time, the viscosity η of the liquid 3 can be calculated based on the flow rate Q and the Hagen-Poiseuille equation. Thus, the viscosity of the liquid 3 can be calculated regardless of the polarity of the liquid 3. For example, even when the liquid 3 is a non-polar solvent such as oil, the characteristic of the liquid 3 can be measured.

When the working liquid 4 and the movable partition wall 21 are not provided (see Comparative Example 1, which will be described later), it is necessary to measure the flow potential generated when the liquid 3 flows and to calculate the flow rate of the liquid 3. However, when the liquid 3 is a non-polar solvent, the generated flow potential is very small and difficult to be measured. Thus, it is difficult to measure the flow rate based on the flow potential, and it is also difficult to calculate the viscosity of the liquid 3.

In the fluid characteristic sensor 1A, the liquid 3 to be measured can be sucked and discharged. Thus, the characteristic of the liquid 3 can be continuously measured over a long period of time. Further, in the fluid characteristic sensor 1A, the liquid 3 used for the measurement can be discharged and then returned to the container 2. Thus, since it is not necessary to sample the liquid 3, the measurement of the liquid 3 can be automatically performed, and a temporal change of the characteristic of the liquid 3 can be measured.

For example, the fluid characteristic sensor 1A can be used to monitor a viscosity of a lubricant. Since the viscosity of the lubricant greatly affects lubricating performance, a preferred embodiment of the present invention can be applied to detection of degradation of the oil by monitoring a variation in viscosity of the lubricant. For example, by attaching the fluid characteristic sensor 1A to the container 2, such as an oil tank, a state of oil degradation can be monitored.

The pressure loss generator 10 is a thin tube with a flow path cross-sectional area smaller than the flow path cross-sectional area of the first flow path 20. With such a configuration, a pressure loss suitable to measure the characteristic of the liquid 3 can be generated in the pressure loss generator 10. In addition, by using the thin tube as the pressure loss generator 10, it is possible to increase the pressure loss to be generated. Due to this, even when the viscosity of a target object is slightly different, a large difference in pressure loss appears, and the flow rate greatly varies. Thus, the viscosity can be measured with high resolution.

The potential measurer 30 includes the first electrode 31, the second electrode 32, and the second flow path 33. The first electrode 31 is an electrode through which the working liquid 4 can pass. The second electrode 32 is disposed at an interval from the first electrode 31, and is an electrode through which the working liquid 4 can pass. The second flow path 33 is disposed between the first electrode 31 and the second electrode 32, and is filled with the working liquid 4. Additionally, the second flow path 33 is a thin tube with a flow path cross-sectional area smaller than the flow path cross-sectional area of the first flow path 20. With such a configuration, the flow potential of the working liquid 4 can be measured.

The working liquid 4 has at least one of a boiling point higher than the boiling point of water and a melting point lower than the melting point of water. With such a configuration, it is possible to operate under an environment at a high temperature being equal to or higher than 100° C. or an environment at a low temperature being equal to or lower than 0° C.

The movable partition wall 21 is defined by or include a gas. The first flow path 20 extends in the gravity direction. In the first flow path 20, an interface 21a between the working liquid 4 and the movable partition wall 21 is higher than an interface 21b between the liquid 3 and the movable partition wall 21. With this configuration, a surface tension acting between the working liquid 4 and the inner wall 20a of the first flow path 20 makes it difficult for the working liquid 4 to naturally flow down in the gravity direction. Thus, the interface 21a between the working liquid 4 and the movable partition wall 21 is maintained. As a result, the fluid characteristic sensor 1A can be stably driven over a long period of time, so that a frequency of maintenance and the number of times of replacement of the fluid characteristic sensor 1A can be reduced.

In addition, the movable partition wall 21 defined by or including a gas includes a larger movable region than a movable partition wall defined by or including a solid. The movable partition wall 21 defined by or including the gas can move in the pressure loss generator 10 and the first flow path 20. As described above, the movable partition wall 21 defined by or including the gas can increase the movable region as compared with the movable partition wall defined by or including the solid, and can increase an introduction amount of the liquid 3 to be measured. Thus, it is possible to flexibly change the introduction amount of the liquid 3.

Further, the pressure loss generated when the movable partition wall 21 defined by or including the gas moves is very small compared to that of the movable partition wall defined by or including the solid, and thus, the influence thereof can be ignored. Further, the movable partition wall 21 defined by or including the gas can reduce a loss due to friction with the inner wall 20a of the first flow path 20 as compared with that of the movable partition wall defined by or including the solid. Thus, it is possible to move the movable partition wall 21 with a smaller pressure than that of the movable partition wall defined by or including the solid.

In addition, the movable partition wall 21 defined by or including the gas has a higher degree of freedom of a measurement target than the movable partition wall defined by or including a liquid. When the movable partition wall is defined by or including the liquid, a liquid having poor solubility with respect to the liquid 3 to be measured and the working liquid 4 is selected for the liquid defining the movable partition wall. On the other hand, the movable partition wall 21 defined by or including the gas defines and functions as a partition wall regardless of types of the liquid 3 and the working liquid 4 as compared with the movable partition wall defined by or including the liquid.

The inner wall 20a of the first flow path 20 has hydrophobicity. A larger surface tension can be obtained due to such a configuration, the interface 21a between the movable partition wall 21 and the working liquid 4 is more reliably maintained even under gravity.

The fluid characteristic sensor 1A includes the calculator 50 that calculates the characteristic of the liquid 3 based on the flow potential measured by the potential measurer 30. With such a configuration, the characteristic of the liquid 3 can be measured by the fluid characteristic sensor 1A alone.

The calculator 50 calculates the flow velocity of the working liquid 4 based on the flow potential measured by the potential measurer 30, and calculates the viscosity of the liquid 3 based on the flow velocity of the working liquid 4. With such a configuration, the flow velocity can be calculated from the flow potential of the working liquid 4, and the viscosity of the liquid 3 can be calculated.

In Preferred Embodiment 1, an example in which the fluid characteristic sensor 1A measures the viscosity of the liquid 3 as the characteristic of the fluid has been described, but the present invention is not limited to this. The fluid characteristic sensor 1A may be capable of measuring the characteristic of the fluid based on the flow potential.

In Preferred Embodiment 1, an example in which the pressure loss generator 10 is a thin tube has been described, but the present invention is not limited thereto. The pressure loss generator 10 may be any element or structure that can generate a pressure loss to the liquid 3. In addition, the thin tube is not limited to a cylindrical or substantially cylindrical shape, and may be, for example, a rectangular or substantially rectangular tube shape.

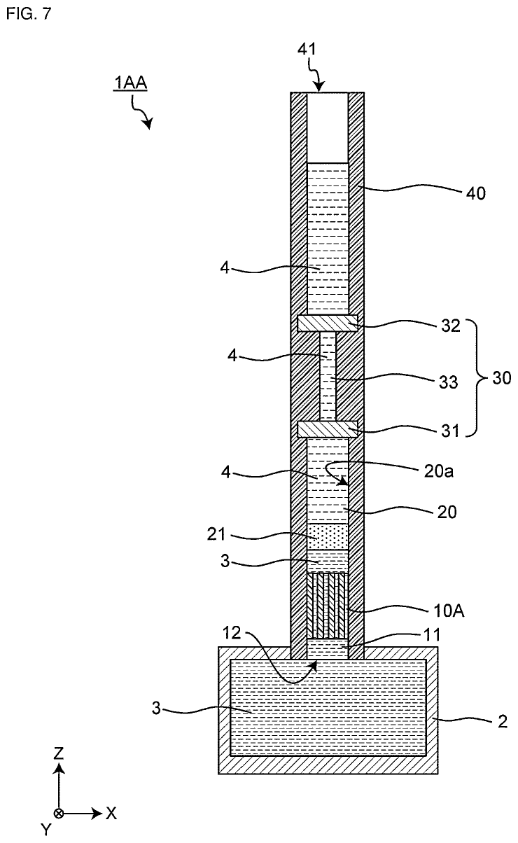

FIG. 7 is a schematic configuration diagram of a fluid characteristic sensor 1AA according to Modification 1 of Preferred Embodiment 1 of the present invention. As illustrated in FIG. 7, in the fluid characteristic sensor 1AA, the pressure loss generator 10A may be a porous body including a plurality of holes. As the porous body, for example, porous silica can be used. Even with such a configuration, it is possible for the pressure loss generator 10A to generate a pressure loss in the liquid 3. In addition, by using the porous body as the pressure loss generator 10, the pressure loss that is generated can be made large as in the case of the thin tube. Due to this, even when the viscosity of a target object is slightly different, a large difference in pressure loss appears, and the flow rate greatly varies. Thus, the viscosity can be measured with high resolution.

In Preferred Embodiment 1, an example in which the movable partition wall 21 is a gas has been described, but the present invention is not limited thereto. It is sufficient that the movable partition wall 21 can partition the liquid 3 and the working liquid 4. For example, the movable partition wall 21 may be defined by or include a solid or a liquid.

Although an example in which the second flow path 33 of the potential measurer 30 is the thin tube has been described in Preferred Embodiment 1, the present invention is not limited thereto. The second flow path 33 may be a flow path in which the flow potential is generated.

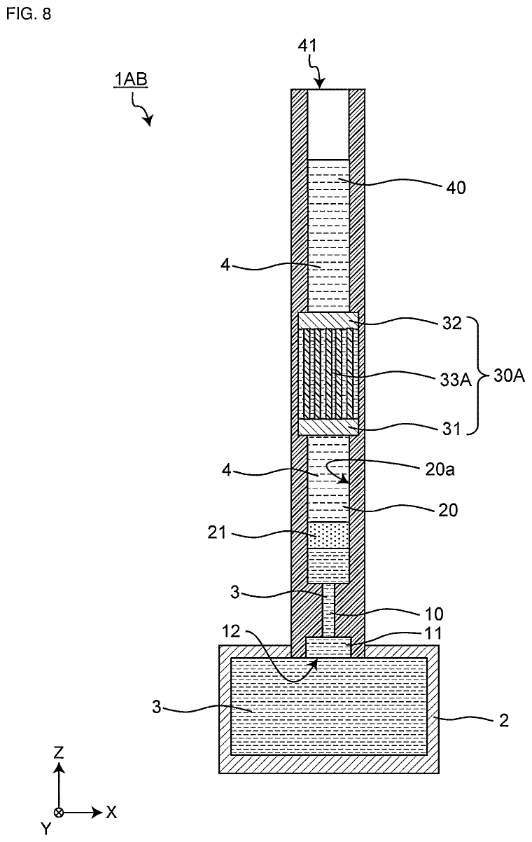

FIG. 8 is a schematic configuration diagram of a fluid characteristic sensor 1AB according to Modification 2 of Preferred Embodiment 1 of the present invention. As illustrated in FIG. 8, in the fluid characteristic sensor 1AB, a second flow path 33A of the potential measurer 30A may be a porous body including a plurality of holes. As the porous body, for example, porous silica can be used. The plurality of holes are designed to have such dimensions that a flow potential can be generated. The porous body may be made of any material as long as the material has an insulation property and defines an electric double layer in a polar solvent. The porous body may be made of, for example, a ceramic material such as alumina or zirconia, or a resin material such as PTFE, PP, or PE. Even with such a configuration, the flow potential of the working liquid 4 can be measured in the potential measurer 30A.

In Preferred Embodiment 1, an example in which the liquid surface of the working liquid 4 positioned at the side of the open end 41 of the working liquid flow path 40 is exposed to the atmosphere side has been described, but the present invention is not limited thereto. The liquid surface of the working liquid 4 does not need to be exposed to the atmosphere side.

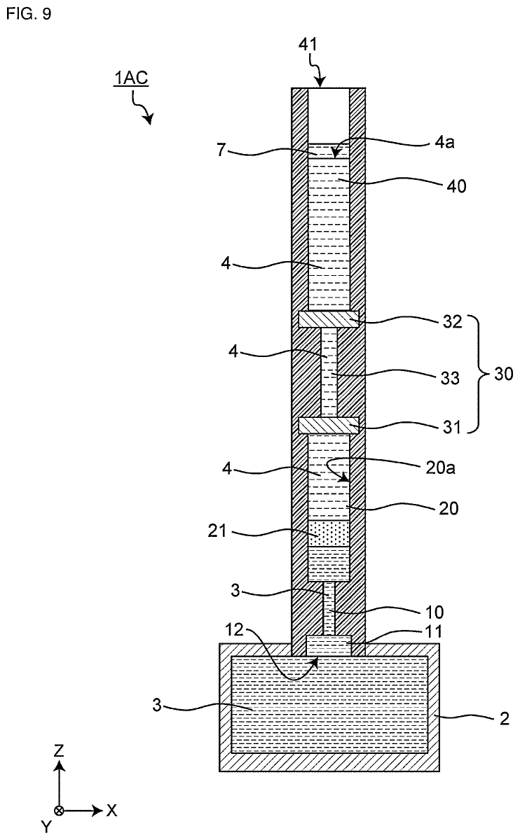

FIG. 9 is a schematic configuration diagram of a fluid characteristic sensor 1AC according to Modification 3 of Preferred Embodiment 1 of the present invention. As illustrated in FIG. 9, in the fluid characteristic sensor 1AC, the liquid surface 4a of the working liquid 4 positioned at the side of the open end 41 of the working liquid flow path 40 is covered with the non-polar solvent 7. With such a configuration, it is possible to reduce or prevent mixture of an intruding object from the outside of the fluid characteristic sensor 1A with the working liquid 4. Thus, a sensor having high environmental resistance can be provided.

The boiling point of the non-polar solvent 7 is preferably higher than the boiling point of the working liquid 4. With such a configuration, it is possible to reduce or prevent a decrease in liquid amount due to gasification of the working liquid 4 at a high temperature.

The non-polar solvent 7 may be, for example, a non-volatile solvent. With such a configuration, it is possible to reduce or prevent a decrease in liquid amount due to volatilization of the working liquid 4.

In Preferred Embodiment 1, an example has been described in which the calculator 50 calculates the viscosity from the flow potential based on the Hagen-Poiseuille equation, but the present invention is not limited thereto. For example, the calculator 50 may prepare a calibration curve in advance and calculate the viscosity from the flow potential by using the calibration curve.

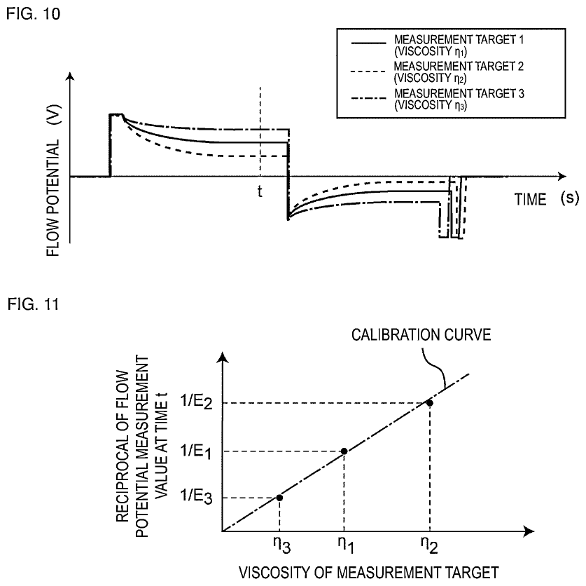

An example of preparation of the calibration curve will be described with reference to FIG. 10 and FIG. 11. FIG. 10 is a graph illustrating an example of changes in flow potentials of three measurement targets measured by the fluid characteristic sensor according to Preferred Embodiment 1 of the present invention. FIG. 11 is a graph illustrating an example of a relationship between the reciprocals of flow potential measurement values at a time t in the graph of FIG. 10 and viscosities of the measurement targets.

As illustrated in FIG. 10, the flow potentials of three measurement targets 1 to 3 having different viscosities from each other are measured. Next, flow potential measurement values E₁, E₂, and E₃ at the time t at which the flow potentials converge are acquired. As illustrated in FIG. 11, the viscosity η of the measurement target is plotted on the horizontal axis, and the reciprocal 1/E of the flow potential measurement value at the time t is plotted on the vertical axis. Thus, the correlation between the reciprocal 1/E of the flow potential measurement value and the viscosity η of the measurement target, that is, a calibration curve can be obtained.

Although an example in which the measurement method includes steps ST1 to ST4 has been described in Preferred Embodiment 1, the measurement method is not limited thereto. These steps ST1 to ST4 may be divided, integrated, deleted, or added, or the order thereof may be changed.

Although an example in which the partition wall 21 is a gas has been described in Preferred Embodiment 1, the present invention is not limited thereto. For example, the partition wall 21 may be a liquid insoluble in the fluid to be measured or the working liquid 4. Alternatively, the partition wall 21 may be a solid that is deformed by receiving a liquid feeding pressure that causes the working liquid 4 to flow, or may be a solid that moves while sliding in the first flow path 20 by receiving a liquid feeding pressure while being in contact with the inner wall 20a of the first flow path 20.

### Preferred Embodiment 2

A fluid characteristic sensor and a measurement method according to Preferred Embodiment 2 of the present invention will be described. In Preferred Embodiment 2, differences from Preferred Embodiment 1 will be mainly described. In Preferred Embodiment 2, elements that are the same as or equivalent to those in Preferred Embodiment 1 are denoted by the same reference signs. Further, in Preferred Embodiment 2, description overlapping with that of Preferred Embodiment 1 will be omitted.

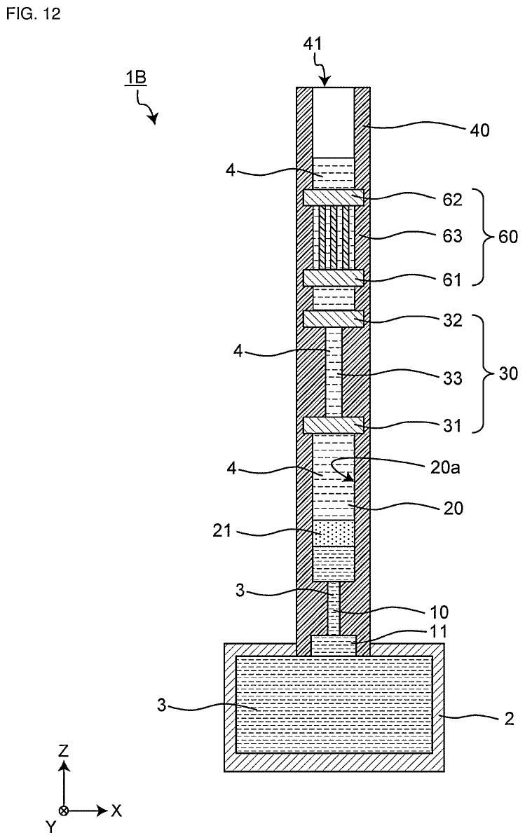

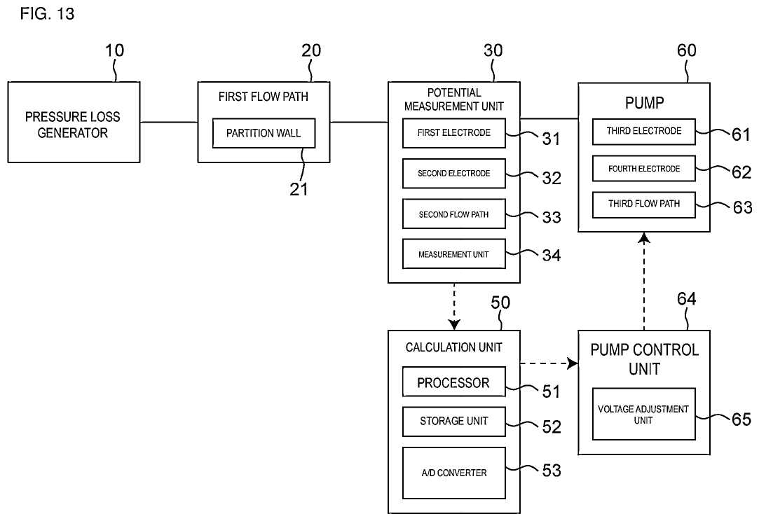

An example of the fluid characteristic sensor according to Preferred Embodiment 2 will be described with reference to FIG. 12 and FIG. 13. FIG. 12 is a schematic configuration diagram of an example of a fluid characteristic sensor 1B according to Preferred Embodiment 2 of the present invention. FIG. 13 is a block diagram illustrating a main configuration of an example of the fluid characteristic sensor 1B according to Preferred Embodiment 2 of the present invention.

Preferred Embodiment 2 is different from Preferred Embodiment 1 in that a pump 60 and a pump controller 64 are provided.

As illustrated in FIG. 12 and FIG. 13, the fluid characteristic sensor 1B includes the pump 60 and the pump controller 64.

### Pump

The pump 60 is connected to the potential measurer 30, and feeds the working liquid 4. In Preferred Embodiment 2, the pump 60 is disposed in the working liquid flow path 40, and feeds the working liquid 4 positioned in the working liquid flow path 40. In the fluid characteristic sensor 1B, suction and discharge of the liquid 3 are performed by switching the liquid feeding direction of the working liquid 4 by the pump 60.

The pump 60 is an electroosmotic flow pump, and includes a third electrode 61, a fourth electrode 62, and a third flow path 63.

The third electrode 61 and the fourth electrode 62 are made of a material through which the working liquid 4 can pass. The third electrode 61 and the fourth electrode 62 are made of, for example, a porous conductive material. As the porous conductive material, a metal material such as, for example, Pt, Cu, Ag, Au, Ni, or stainless steel can be used. In Preferred Embodiment 2, each of the third electrode 61 and the fourth electrode 62 is made of a metal mesh having a flat plate shape and including two opposing main surfaces.

The third electrode 61 and the fourth electrode 62 are disposed so as to be spaced apart from each other. Specifically, the third electrode 61 and the fourth electrode 62 are disposed to face each other with an interval therebetween in the flow direction (Z direction) of the working liquid 4. Additionally, the main surfaces of the third electrode 61 and the fourth electrode 62 are disposed in a direction intersecting the flow direction (Z direction) of the working liquid 4.

The third flow path 63 is disposed between the third electrode 61 and the fourth electrode 62, and is filled with the working liquid 4. In Preferred Embodiment 2, the third flow path 63 is a porous body through which the working liquid 4 flows. Specifically, the third flow path 63 is a porous body including a plurality of holes. The plurality of holes are designed to have such dimensions that a flow potential can be generated. As the porous body, for example, porous silica can be used. The porous body may be made of any material as long as the material has an insulation property and defines an electric double layer in a polar solvent. The porous body may be made of, for example, a ceramic material such as alumina or zirconia, or a resin material such as PTFE, PP, or PE.

The porous body defining the third flow path 63 includes one end and another end. The third electrode 61 is disposed at the one end of the porous body. The fourth electrode 62 is disposed at the other end of the porous body.

### Pump Controller

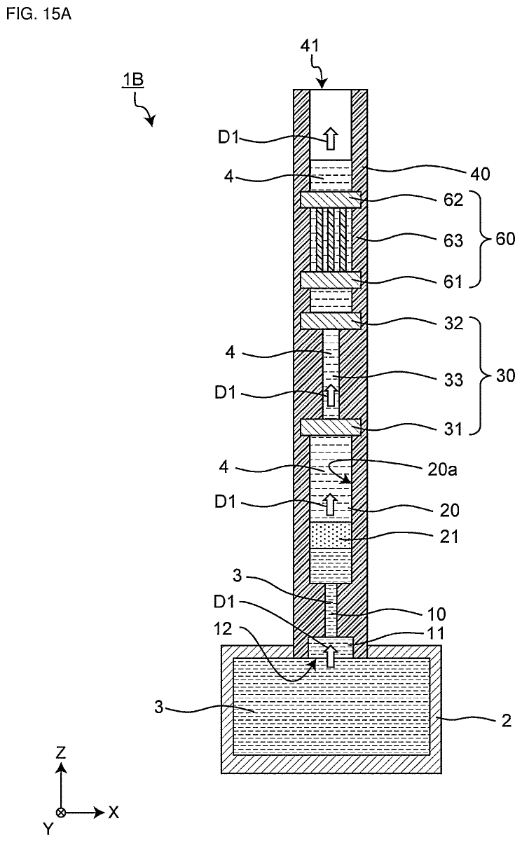

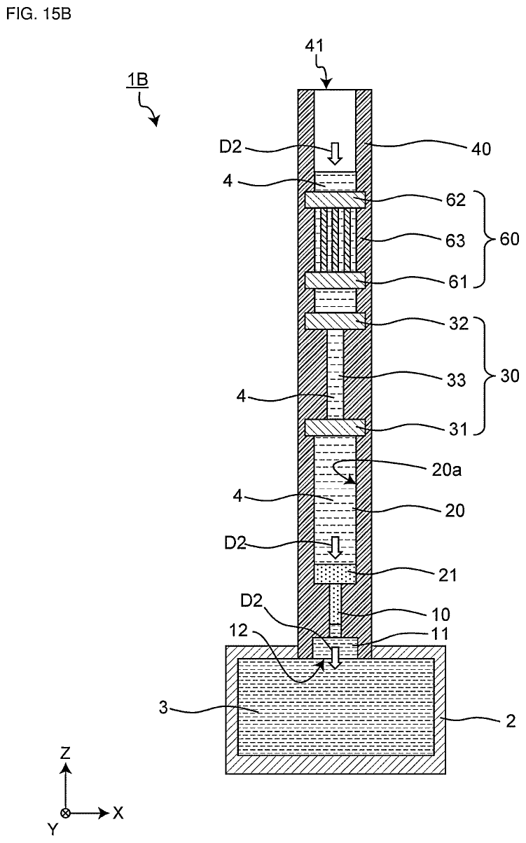

The pump controller 64 controls a liquid feeding direction and a liquid feeding pressure of the pump 60. The liquid feeding direction includes the first direction D1 and the second direction D2. The first direction D1 is a direction in which the liquid 3 is sucked, and is a direction from the pressure loss generator 10 toward the pump 60 (see FIG. 15A). The second direction D2 is opposite to the first direction D1, and is a direction from the pump 60 toward the pressure loss generator 10. The liquid feeding pressure means a pressure for feeding the working liquid 4 by the pump 60.

The pump controller 64 controls the liquid feeding direction and the liquid feeding pressure of the pump 60 by controlling an application voltage to be applied to the pump 60. Specifically, the pump controller 64 includes a voltage adjuster 65 that adjusts the application voltage to the pump 60. The voltage adjuster 65 adjusts a magnitude of the application voltage to be applied to the pump 60 and a positive or negative polarity of the application voltage. For example, the voltage adjuster 65 is a circuit that adjusts a voltage, and includes a semiconductor element or the like.

The pump controller 64 controls the liquid feeding pressure of the pump 60 by adjusting the magnitude of the application voltage to be applied to the pump 60 by the voltage adjuster 65. In addition, the pump controller 64 controls the liquid feeding direction of the pump 60 by adjusting the positive or negative polarity of the application voltage to be applied to the pump 60 by the voltage adjuster 65.

In Preferred Embodiment 2, the pump controller 64 controls the liquid feeding direction and the liquid feeding pressure of the pump 60 based on the measurement value of the flow potential measured by the potential measurer 30.

### Operation

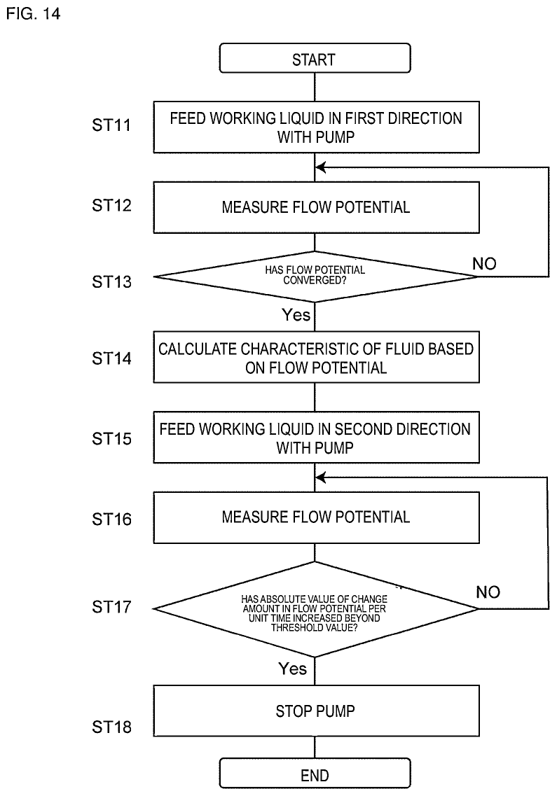

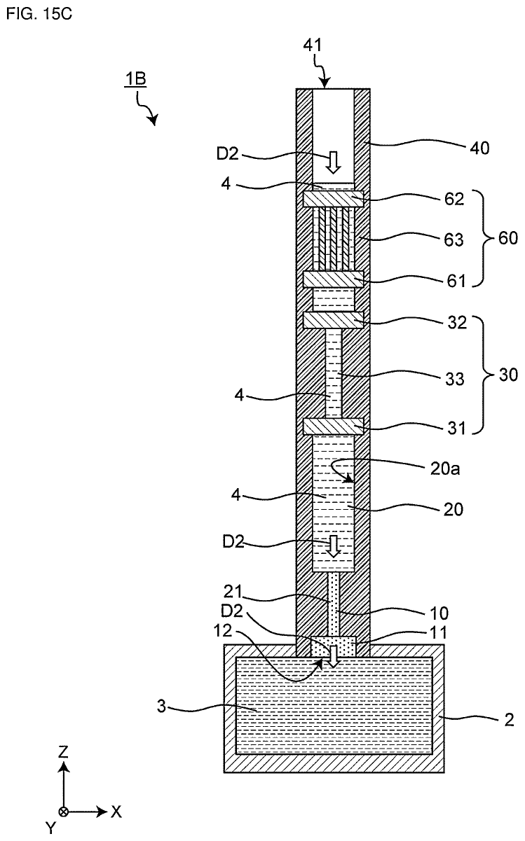

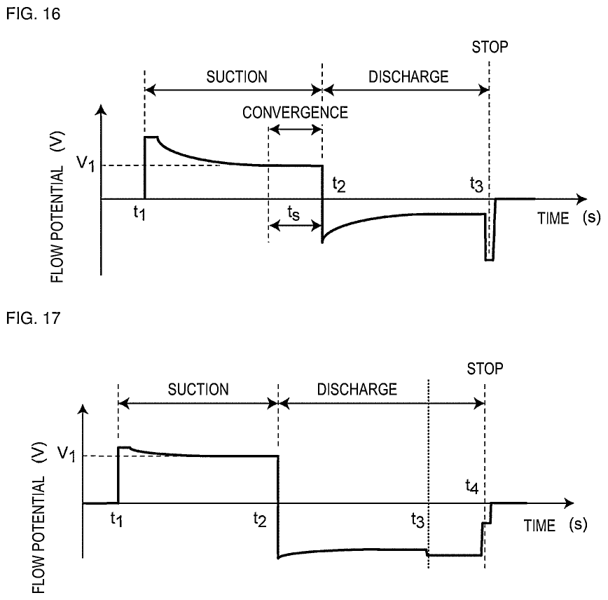

An example of an operation of the fluid characteristic sensor 1B, that is, a measurement method will be described with reference to FIG. 14 to FIG. 16. FIG. 14 is a flowchart of the example of the measurement method according to Preferred Embodiment 2 of the present invention. FIGS. 15A to 15C are schematic diagrams illustrating the example of the operation of the fluid characteristic sensor 1B according to Preferred Embodiment 2 of the present invention. FIG. 16 is a graph illustrating an example of a change in flow potential measured by the fluid characteristic sensor 1B according to Preferred Embodiment 2 of the present invention. The operation will be described with respect to an example in which a viscosity is measured as a characteristic of the liquid 3 to be measured.

As illustrated in FIG. 14 and FIG. 15A, in step ST11, the working liquid 4 is fed in the first direction D1 by the pump 60. In step ST11, the pump controller 64 controls the application voltage to be applied to the pump 60 by the voltage adjuster 65. For example, the pump controller 64 performs control so as to apply an application voltage of about +12 V to the pump 60. Thus, the pump 60 feeds the working liquid 4 in the first direction D1. As a result, the liquid 3 stored in the container 2 is sucked from the inflow/outflow port 12.

Returning to FIG. 14, in step ST12, a flow potential of the working liquid 4 is measured by the potential measurer 30. To be specific, in the potential measurer 30, the measurer 34 measures a flow potential generated by the working liquid 4 flowing in the first direction D1 through the second flow path 33 disposed between the first electrode 31 and second electrode 32.

In step ST13, the calculator 50 determines whether or not the flow potential has converged. As illustrated in FIG. 16, the flow potential increases with the start of suction at a time t₁, and decreases and converges with the lapse of time. The convergence of the flow potential is determined based on the threshold value of the change amount in flow potential per unit time tₛ. For example, when the change amount in the flow potential for about 10 seconds is within about ±0.02 V, the calculator 50 may determine that the flow potential has converged. The unit time tₛ is not limited to about 10 seconds, and may be set to any value. Additionally, the threshold value of the change amount in the flow potential is not limited to about ±0.02 V, and may be set to any value.

Returning to FIG. 14, in step ST13, when the flow potential has converged, the processing proceeds to step ST14. When the flow potential has not converged, the processing returns to step ST12.

In Step ST14, a characteristic of the liquid 3 to be measured is calculated by the calculator 50 based on the measured flow potential. To be specific, the calculator 50 acquires a measurement value when the flow potential has converged, that is, a convergence value V₁ of the flow potential. The calculator 50 calculates the viscosity of the liquid 3 based on the convergence value V₁ of the flow potential.

As illustrated in FIG. 14 and FIG. 15B, in step ST15, the working liquid 4 is fed in the second direction D2 by the pump 60. In step ST15, the pump controller 64 controls an application voltage to be applied to the pump 60 by the voltage adjuster 65. For example, the pump controller 64 performs control so as to apply an application voltage of about −12 V to the pump 60. Thus, the pump 60 feeds the working liquid 4 in the second direction D2. As a result, the liquid 3 in the fluid characteristic sensor 1B is discharged into the container 2. By making the magnitude of an application voltage in suction and the magnitude of an application voltage in discharge identical or substantially identical, the liquid feeding pressure in suction and the liquid feeding pressure in discharge can be made identical or substantially identical.

In Preferred Embodiment 2, the pump controller 64 receives information about a switching timing of the liquid feeding direction of the pump 60 from the calculator 50. Examples of the information about the switching timing of the liquid feeding direction include a determination result of convergence of the flow potential. The pump controller 64 receives the determination result of the convergence of the flow potential from the calculator 50, and switches the liquid feeding direction of the pump 60 based on the determination result.

In step ST16, the flow potential of the working liquid 4 is measured by the potential measurer 30. To be specific, in the potential measurer 30, the measurer 34 measures the flow potential generated by the working liquid 4 flowing in the second direction D2 through the second flow path 33 disposed between the first electrode 31 and second electrode 32.

In step ST17, the calculator 50 determines whether or not the absolute value of a change amount in flow potential per unit time has increased beyond a threshold value. The calculator 17 determines whether the discharge of the liquid 3 has been completed by determining whether or not the absolute value of the change amount in flow potential per unit time has increased beyond the threshold value. As illustrated in FIG. 15C, when the liquid 3 is discharged, the movable partition wall 21 defined by or including a gas is positioned in the pressure loss generator 10. Thus, since a pressure loss in the pressure loss generator 10 rapidly decreases, a flow velocity of the working liquid 4 moving in the second direction D2 rapidly increases. When the flow velocity of the working liquid 4 rapidly increases, the absolute value of the flow potential measured by the potential measurer 30 also rapidly increases.

As illustrated in FIG. 16, when the liquid feeding direction of the pump 60 is switched from the first direction D1 to the second direction D2 at a time t₂, a flow direction of the working liquid 4 is also reversed. Thus, the flow potential measured by the potential measurer 30 is also inverted from positive to negative. The absolute value of the measurement value of the flow potential decreases with the lapse of time and converges. Then, as illustrated in FIG. 15C, when the liquid 3 is discharged, the flow velocity of the working liquid 4 flowing in the second direction D2 rapidly increases, and the absolute value of the flow potential measured by the potential measurer 30 rapidly increases. For example, the calculator 50 may set a unit time to about 1 second, and may set the threshold value to about 0.1 V. In a case where the absolute value of the change amount in flow potential has increased beyond about 0.1 V in one second, the calculator 50 determines that the absolute value of the change amount in flow potential has increased beyond the threshold value.

Returning to FIG. 14, in step ST17, in a case where the absolute value of the change amount in flow potential per unit time has exceeded the threshold value, the processing proceeds to step ST18. In a case where the absolute value of the change amount in flow potential per unit time has not exceeded the threshold value, the processing returns to step ST16.

In step ST18, the pump 60 is stopped by the pump controller 64. To be specific, the pump controller 64 sets an application voltage to be applied to the pump 60 by the voltage adjuster 65 to 0 V. By setting the application voltage to 0 V, a liquid feeding pressure of the pump 60 can be set to 0. That is, driving of the pump 60 can be stopped.

In Preferred Embodiment 2, the pump controller 64 receives timing information for stopping the pump 60 from the calculator 50. The timing information for stopping the pump 60 is, for example, a determination result as to whether or not the absolute value of the change amount in flow potential per unit time has exceeded the threshold value. The pump controller 64 receives the determination result of the change amount in flow potential from the calculator 50, and stops the pump 60 at a time t₃ based on the determination result.

As described above, in the measurement method using the fluid characteristic sensor 1B, the viscosity can be measured as the characteristic of the liquid 3 by performing steps ST11 to ST18.

### Advantageous Effects

According to the fluid characteristic sensor 1B of Preferred Embodiment 2, the following advantageous effects can be obtained.

The fluid characteristic sensor 1B includes the pump 60 that is connected to the potential measurer 30 and that feeds the working liquid 4. With such a configuration, the working liquid 4 can be easily and appropriately fed.

The pump 60 is an electroosmotic flow pump, and includes the third electrode 61, the fourth electrode 62, and the third flow path 63. The third electrode 61 is an electrode through which the working liquid 4 can pass. The fourth electrode 62 is an electrode that is disposed at an interval from the third electrode 61 and through which the working liquid 4 can pass. The third flow path 63 is disposed between the third electrode 61 and the fourth electrode 62, and is filled with the working liquid 4. The third flow path 63 has a porous body including a plurality of holes. With such a configuration, the pump 60 is driven with a DC voltage, and the liquid feeding direction can be easily switched by reversing the polarity of the application voltage. In addition, since the structure is simple and small, the structure can be easily incorporated into the fluid characteristic sensor 1B, and a degree of freedom in design is improved.

The fluid characteristic sensor 1B includes the pump controller 64 that controls the liquid feeding direction of the pump 60. The liquid feeding direction includes the first direction D1 from the pressure loss generator 10 toward the pump 60, and the second direction D2 that is opposite to the first direction D1 and that is from the pump 60 toward the pressure loss generator 10. With such a configuration, the liquid feeding direction of the pump 60 can be easily controlled.

The pump controller 64 controls the liquid feeding direction of the pump 60 based on the measurement value of the flow potential measured by the potential measurer 30. With such a configuration, the liquid feeding direction of the pump 60 can be adjusted at an appropriate timing.

After the measurement value of the flow potential converges when the liquid feeding direction is the first direction D1, the pump controller 64 switches the liquid feeding direction to the second direction D2. In addition, the pump controller 64 stops the pump 60 when the liquid feeding direction is the second direction D2 and the absolute value of the change amount in flow potential per unit time increases beyond the threshold value. With such a configuration, the liquid feeding direction can be switched from the first direction D1 to the second direction D2 at a more appropriate timing. Further, the pump 60 can be stopped at a more appropriate timing.

Although an example in which the pump 60 is an electroosmotic pump has been described in Preferred Embodiment 2, the pump 60 is not limited thereto. The pump 60 may be a pump capable of feeding the working liquid 4.

Although an example in which the fluid characteristic sensor 1B includes the pump controller 64 has been described in Preferred Embodiment 2, the present invention is not limited thereto. For example, the pump controller 64 is not necessarily provided, and may be included in a control device that controls the fluid characteristic sensor 1B.

Although an example in which the pump controller 64 controls both the liquid feeding direction and the liquid feeding pressure of the pump 60 has been described in Preferred Embodiment 2, the present invention is not limited thereto. The pump controller 64 only needs to be able to control at least the liquid feeding direction.

Although an example in which the measurement method includes steps ST11 to ST18 has been described in Preferred Embodiment 2, the measurement method is not limited thereto. These steps ST11 to ST18 may be divided, integrated, deleted, or added, or the order thereof may be changed.

In Preferred Embodiment 2, an example has been described in which the calculator 50 determines whether or not the absolute value of the change amount in flow potential per unit time tₛ has increased beyond the threshold value in step ST17, but the present invention is not limited thereto. In step ST17, it is only necessary to be able to determine the end of the discharge of the liquid 3. For example, in step ST17, the calculator 50 may determine whether or not the absolute value of the flow potential has increased beyond a threshold value.

In Preferred Embodiment 2, an example has been described in which the calculator 50 determines whether or not the absolute value of the change amount in flow potential per unit time tₛ has decreased beyond the threshold value in step ST17A, but the present invention is not limited thereto. In step ST17A, it is only necessary to determine whether the working liquid 4 flows into the pressure loss generator 10. For example, in step ST17A, the calculator 50 may determine whether or not the absolute value of the flow potential has decreased beyond a threshold value.

FIG. 17 is a graph illustrating another example of a change in flow potential measured by the fluid characteristic sensor 1B according to Preferred Embodiment 2 of the present invention. As illustrated in FIG. 17, the absolute value of the change amount in flow potential per unit time does not exceed the threshold value in some cases even after the liquid 3 is completely discharged at the time t₃. In such a case, when the working liquid 4 is continuously fed to the second direction D2 by the pump 60, the working liquid 4 flows out to the outside of the fluid characteristic sensor 1B.

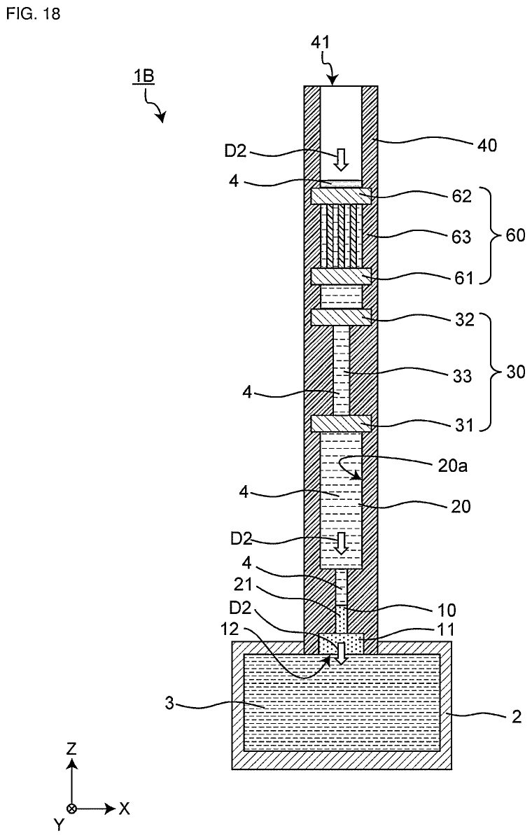

FIG. 18 is a schematic diagram illustrating another example of the operation of the fluid characteristic sensor 1B according to Preferred Embodiment 2 of the present invention. As illustrated in FIG. 18, when the pump 60 continues to feed the working liquid 4 in the second direction D2 even after the liquid 3 is completely discharged, the working liquid 4 flows in the pressure loss generator 10. When the working liquid 4 flows in the pressure loss generator 10, the pressure loss rapidly increases in the pressure loss generator 10. Due to this, the flow velocity (flow rate) of the working liquid 4 rapidly decreases. Thus, as illustrated in FIG. 17, the absolute value of the flow potential rapidly decreases at a time t₄ at which the working liquid 4 flows in the pressure loss generator 10 and the pressure loss rapidly increases. By stopping the pump 60 at the timing at which the absolute value of the flow potential rapidly decreases, the flow out of the working liquid 4 to the outside of the fluid characteristic sensor 1B can be reduced or prevented.

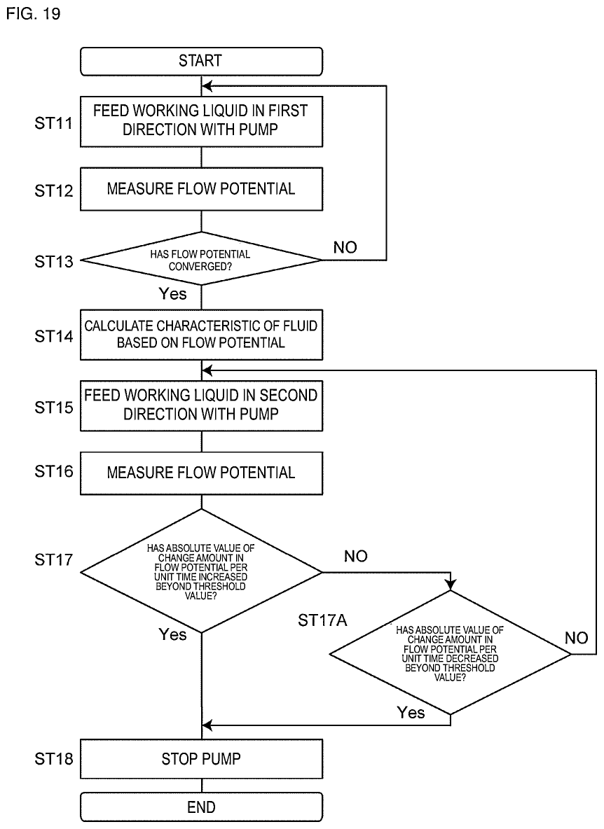

FIG. 19 is a flowchart of an example of a measurement method according to Modification 4 of Preferred Embodiment 2 of the present invention. Except for step ST17A illustrated in FIG. 19, steps ST11 to ST18 are the same or substantially the same as steps ST11 to ST18 illustrated in FIG. 14, and thus, description thereof will be omitted. As illustrated in FIG. 19, in a case of No in step ST17, the processing proceeds to step ST17A.

In step ST17A, the calculator 50 determines whether or not the absolute value of the change amount in flow potential per unit time has decreased beyond a threshold value. For example, the calculator 50 may set a unit time to about 1 second, and may set the threshold value to about 0.1 V. In a case where the absolute value of the change amount in flow potential has decreased beyond about 0.1 V in one second, the calculator 50 determines that the absolute value has decreased beyond the threshold value.

In step ST17A, in a case where the absolute value of the change amount in flow potential per unit time has decreased beyond the threshold value, the processing proceeds to step ST18. In a case where the absolute value of the change amount in flow potential per unit time has not decreased beyond the threshold value, the processing returns to step ST16.

With such a configuration, even when the control of step ST17 does not function, the flow out of the working liquid 4 to the outside of the fluid characteristic sensor 1B can be reduced or prevented by executing step ST17A.

When the measurement method illustrated in FIG. 19 is performed, the movable partition wall 21 preferably has a volume larger than a flow path volume of the pressure loss generator 10. This makes it possible to more reliably measure a decrease in the absolute value of the change amount in flow potential.

In Preferred Embodiment 2, an example in which the liquid feeding direction of the working liquid 4 is changed from the first direction D1 to the second direction D2 when the flow potential converges has been described, but the present invention is not limited thereto. For example, the calculator 50 may calculate a suction amount of the liquid 3 based on the flow potential measured when the liquid 3 to be measured is sucked. The pump controller 64 may control the pump 60 based on the suction amount of the liquid 3 calculated by the calculator 50 to change the liquid feeding direction of the working liquid 4 from the first direction D1 to the second direction D2. For example, the pump controller 64 may change the liquid feeding direction of the working liquid 4 from the first direction D1 to the second direction D2 before the suction amount of the liquid 3 exceeds the flow path volume from the potential measurer 30 to the inflow/outflow port 12. With such a configuration, the entrance of the liquid 3 to the potential measurer 30 or the pump 60 can be reduced or prevented. Accordingly, a decrease in measurement accuracy and a failure due to contamination of the fluid characteristic sensor 1B by the liquid 3 can be reduced or prevented.

### Preferred Embodiment 3

A fluid characteristic sensor and a measurement method according to Preferred Embodiment 3 of the present invention will be described. In Preferred Embodiment 3, differences from Preferred Embodiment 2 will be mainly described. In Preferred Embodiment 3, elements that are the same as or equivalent to those in Preferred embodiment 2 are denoted by the same reference signs. Further, in Preferred Embodiment 3, description overlapping with that of Preferred Embodiment 2 will be omitted.

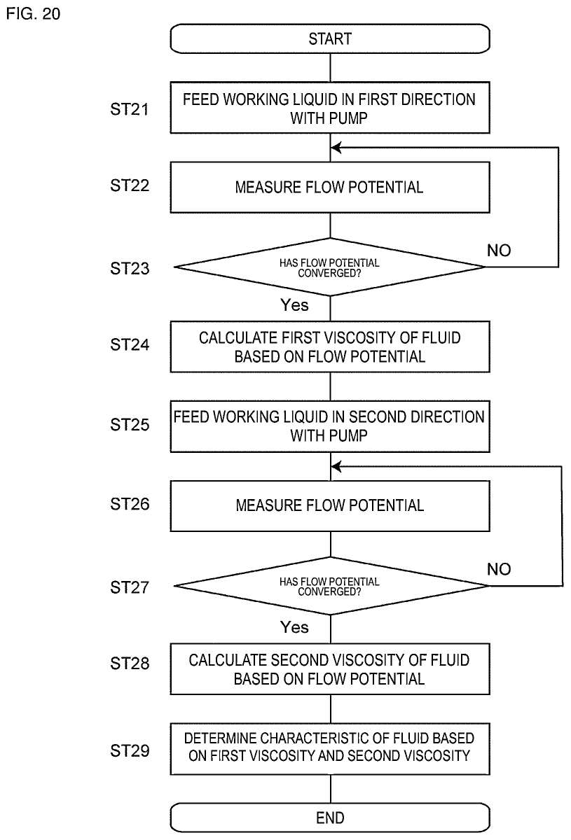

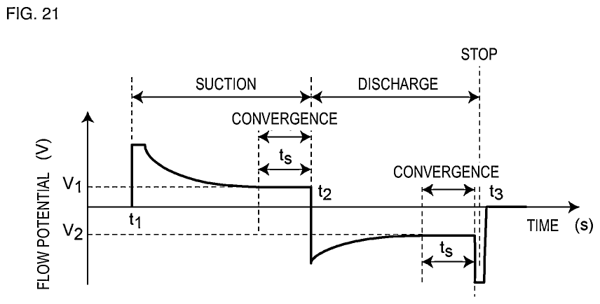

An example of a measurement method using the fluid characteristic sensor according to Preferred Embodiment 3 will be described with reference to FIG. 20 and FIG. 21. FIG. 20 is a flowchart of the example of the measurement method according to Preferred Embodiment 3 of the present invention. FIG. 21 is a graph illustrating an example of a change in flow potential measured by the fluid characteristic sensor according to Preferred Embodiment 3 of the present invention.

Preferred Embodiment 3 is different from Preferred Embodiment 2 in that a first viscosity of the liquid 3 when the liquid 3 is sucked and a second viscosity of the liquid 3 when the liquid 3 is discharged are measured, and a characteristic of the liquid 3 is determined based on the first viscosity and the second viscosity.

In the measurement method according to Preferred Embodiment 3, the calculator 50 calculates the first viscosity of the liquid 3 based on the measurement value of the flow potential when the liquid 3 is sucked, and calculates the second viscosity of the liquid 3 based on the measurement value of the flow potential when the liquid 3 is discharged. Additionally, the calculator 50 determines the characteristic of the liquid 3 based on the first viscosity and the second viscosity. Specifically, in the measurement method according to Preferred Embodiment 3, it is determined whether or not the liquid 3 is a fluid exhibiting thixotropy.

More specifically, some fluids have a property of decreasing in viscosity with the lapse of time while the fluid is being flowed at a constant shear rate and then returning to an original high viscosity state when the fluid is caused to stop flowing to be kept stationary for a while. This property is called thixotropy.

A shear rate when the Hagen-Poiseuille flow is generated in a circular or substantially circular tube is represented by γ=4Q/πR\hat{ }3 (γ: shear rate, Q: flow rate, R: tube radius). Thus, the shear rate increases in inverse proportion to the third power of the tube radius.

In the fluid characteristic sensor according to Preferred Embodiment 3, when the liquid 3 to be measured passes through the pressure loss generator 10, a large shear rate is applied. Thus, when the liquid 3 is a fluid exhibiting thixotropy, the viscosity changes with the lapse of time particularly when the liquid 3 passes through the pressure loss generator 10. A period of time required for the liquid 3 to pass through the pressure loss generator 10 is assumed to be tₐ seconds, the liquid 3 flows at a certain shear rate for tₐ seconds, during which the viscosity changes with the lapse of time. It is assumed that the viscosity immediately before the liquid 3 enters the pressure loss generator 10 is η₁₁ and the viscosity immediately after the liquid 3 passes through the pressure loss generator 10 (after tₐ seconds) is η₁₂, an apparent viscosity obtained from the measurement value of the flow potential in suction has a value between η₁₁ and η₁₂ (not necessarily an average value).

When the liquid 3 finishes passing through the pressure loss generator 10, the shear rate applied to the liquid 3 is significantly reduced, and thus, the viscosity of the liquid 3 tries to return to η₁₁. However, when the liquid feeding direction is reversed before the viscosity returns to η₁₁, and the liquid enters the pressure loss generator 10 again, the apparent viscosity obtained from the measurement value of the flow potential in discharge has a value smaller than the apparent viscosity obtained in suction.

Thus, when the first viscosity of the liquid 3 obtained from the measurement value of the flow potential in suction and the second viscosity obtained from the measurement value of the flow potential in discharge are compared, and the values are different, it can be determined that the liquid 3 exhibits thixotropy.

An example of the measurement method according to Preferred Embodiment 3 will be described with reference to FIG. 20 and FIG. 21. Steps ST21 to ST25 illustrated in FIG. 20 are the same or substantially the same as steps ST11 to ST15 of Preferred Embodiment 2, and thus, detailed description thereof will be omitted.

As illustrated in FIG. 20, by performing steps ST21 to ST24, the liquid 3 is sucked, and the first viscosity of the liquid 3 is calculated as a first characteristic of the liquid 3 to be measured.

In step ST25, the working liquid 4 is fed in the second direction D2 by the pump 60. In step ST35, the pump controller 64 controls a voltage to be applied to the pump 60 by the voltage adjuster 65. For example, the pump controller 64 performs control so as to apply an application voltage of about −12 V to the pump 60. Thus, the pump 60 feeds the working liquid 4 in the second direction D2. As a result, the liquid 3 in the fluid characteristic sensor 1B is discharged into the container 2. By setting the magnitude of a voltage to be applied to the pump 60 during liquid feeding in the first direction D1 and the magnitude of a voltage to be applied to the pump 60 during liquid feeding in the second direction D2 to be the same or substantially the same, the liquid feeding pressures during liquid feeding in the first direction D1 and the second direction D2 can be set to be the same or substantially the same.

In step ST26, the flow potential of the working liquid 4 is measured by the potential measurer 30. To be specific, in the potential measurer 30, the measurer 34 measures the flow potential generated by the working liquid 4 flowing in the second direction D2 through the second flow path 33 disposed between the first electrode 31 and second electrode 32. As illustrated in FIG. 21, the flow potential is reversed when the discharge of the liquid 3 is started at the time t₂. Similar to the suction of the liquid 3, the absolute value of the flow potential decreases and converges with the lapse of time.

Referring back to FIG. 20, in step ST27, the calculator 50 determines whether or not the flow potential has converged. The convergence of the flow potential is determined based on the threshold value of the change amount in flow potential per unit time tₛ, as in step ST23. For example, when the change amount in the flow potential for 10 seconds is within about ±0.02 V, the calculator 50 may determine that the flow potential has converged. The unit time tₛ is not limited to about 10 seconds, and may be set to any value. Additionally, the threshold value of the change amount in the flow potential is not limited to about ±0.02 V, and may be set to any value.

In step ST27, in a case where the flow potential has converged, the processing proceeds to step ST28. In a case where the flow potential has not converged, the processing returns to step ST26.

In step ST28, a second characteristic of the liquid 3 is calculated by the calculator 50 based on the measured flow potential. To be specific, the calculator 50 acquires a measurement value when the flow potential has converged, that is, a convergence value V₂ of the flow potential. The calculator 50 calculates the second viscosity of the liquid 3 based on the convergence value V₂ of the flow potential.

In step ST29, the characteristic of the liquid 3 is determined by the calculator 50 based on the first viscosity and the second viscosity. Specifically, the calculator 50 compares the first viscosity and the second viscosity. When the first viscosity and the second viscosity are different from each other, the calculator 50 determines that the liquid 3 is a fluid that exhibits thixotropy. When the first viscosity and the second viscosity are equal or substantially equal to each other, the calculator 50 determines that the liquid 3 is a fluid that does not exhibit thixotropy.

Thus, in the measurement method according to Preferred Embodiment 3, the characteristic of the liquid 3 can be determined by performing steps ST21 to ST29. Specifically, in the measurement method of Preferred Embodiment 3, it is possible to determine whether or not the liquid 3 is a fluid that exhibits thixotropy.

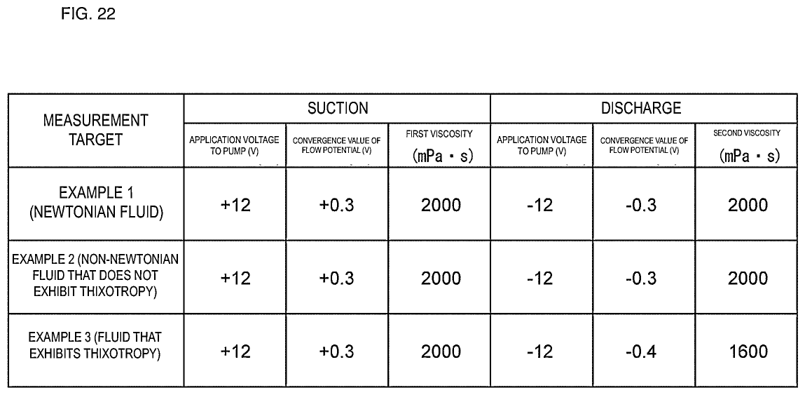

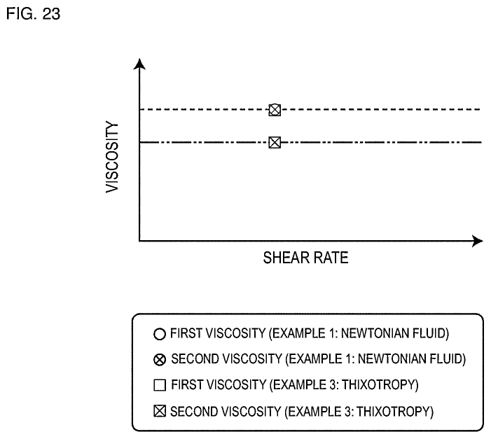

FIG. 22 is a table showing an example of measurement conditions and measurement results of Examples 1 to 3. FIG. 23 is a graph illustrating an example of relationships between viscosities and shear rates in Examples 1 and 3. In Examples 1 to 3 shown in FIG. 22 and illustrated in FIG. 23, the first viscosity of the liquid 3 in suction and the second viscosity of the liquid 3 in discharge were measured by using the fluid characteristic sensor according to Preferred Embodiment 3. In Examples 1 to 3, a Newtonian fluid, a non-Newtonian fluid that does not exhibit thixotropy, and a fluid that exhibits thixotropy were used as the liquid 3 to be measured, respectively. Examples 1 to 3 have the same or substantially the same conditions except for the types of the liquid 3.

As shown in FIG. 22 and illustrated in FIG. 23, in Examples 1 and 2, the first viscosity of the liquid 3 in suction is equal or substantially equal to the second viscosity of the liquid 3 in discharge. On the other hand, in Example 3, the second viscosity of the liquid 3 in discharge is lower than the first viscosity of the liquid 3 in suction. As described above, in the Newtonian fluid in Example 1 and the non-Newtonian fluid that does not exhibit thixotropy in Example 2, the first viscosity in suction is equal or substantially equal to the second viscosity in discharge. On the other hand, in the fluid that exhibits thixotropy in Example 3, when the first viscosity in suction and the second viscosity in discharge are different from each other, the first viscosity and the second viscosity are different from each other. Thus, by comparing the first viscosity and the second viscosity that are measured by using the fluid characteristic sensor according to Preferred Embodiment 3, it is possible to determine whether or not the liquid 3 to be measured is a fluid exhibiting thixotropy.

### Advantageous Effects

According to the fluid characteristic sensor of Preferred Embodiment 3, the following advantageous effects can be obtained.

In the fluid characteristic sensor according to Preferred Embodiment 3, the calculator 50 calculates the first viscosity of the liquid 3 based on the measurement value of the flow potential when the liquid feeding direction is the first direction D1, and calculates the second viscosity of the liquid 3 based on the measurement value of the flow potential when the liquid feeding direction is the second direction D2. The calculator 50 determines the characteristic of the liquid 3 based on the first viscosity and the second viscosity. With such a configuration, viscosities can be measured in suction and discharge of the liquid 3. Thus, a preferred embodiment of the present invention can be applied to determination of the type of the liquid 3. For example, the calculator 50 can determine whether or not the liquid 3 is a fluid exhibiting thixotropy based on the first viscosity and the second viscosity.

Although an example in which the measurement method includes steps ST21 to ST29 has been described in Preferred Embodiment 3, the present invention is not limited thereto. These steps ST21 to ST29 may be divided, integrated, deleted, or added, or the order thereof may be changed.

In Preferred Embodiment 3, an example has been described in which, in step ST29, the liquid 3 is determined to be a fluid that does not exhibit thixotropy when the first viscosity and the second viscosity are equal or substantially equal to each other, and the liquid 3 is determined to be a fluid that exhibits thixotropy when the first viscosity and the second viscosity are different from each other, but the present invention is not limited thereto. For example, the calculator 50 may calculate a difference between the first viscosity and the second viscosity, and may determine the characteristic of the liquid 3 based on the difference and a predetermined threshold value. For example, when the difference exceeds the predetermined threshold value, the calculator 50 may determine that the liquid 3 is a fluid exhibiting thixotropy. When the difference does not exceed the predetermined threshold value, the calculator 50 may determine that the liquid 3 is a fluid that does not exhibit thixotropy.

### Preferred Embodiment 4

A fluid characteristic sensor and a measurement method according to Preferred Embodiment 4 of the present invention will be described. In Preferred Embodiment 4, differences from Preferred Embodiment 3 will be mainly described. In Preferred Embodiment 4, elements that are the same as or equivalent to those in Preferred Embodiment 3 are denoted by the same reference signs. Further, in Preferred Embodiment 4, description overlapping with that of Preferred Embodiment 3 will be omitted.

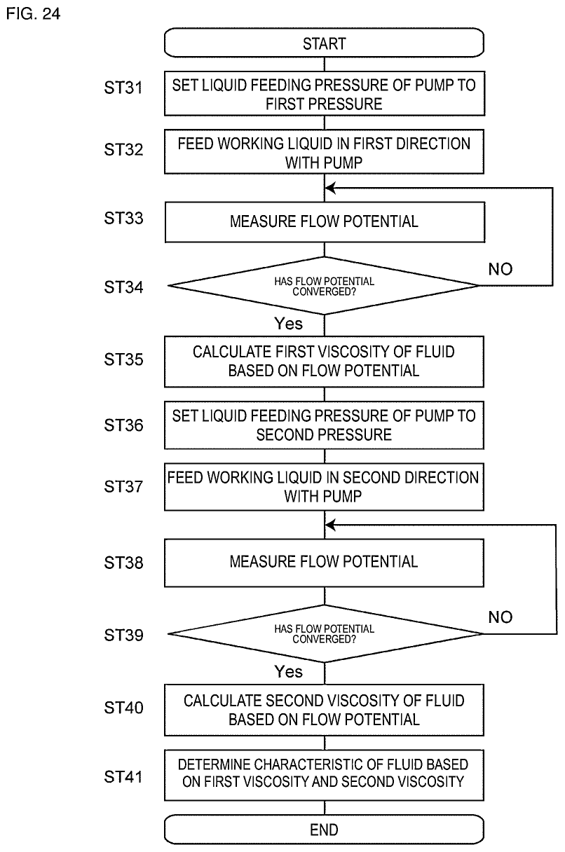

An example of the measurement method using the fluid characteristic sensor according to Preferred Embodiment 4 will be described with reference to FIG. 24. FIG. 24 is a flowchart of the example of the measurement method according to Preferred Embodiment 4 of the present invention.

Preferred Embodiment 4 is different from Preferred Embodiment 3 in that the liquid feeding pressure in sucking the liquid 3 and the liquid feeding pressure in discharging the liquid 3 are different from each other.

In the measurement method according to Preferred Embodiment 4, the characteristic of the liquid 3 is determined based on the first viscosity and the second viscosity of the liquid 3 that are measured by setting the liquid feeding pressure in sucking the liquid 3 to be different from the liquid feeding pressure in discharging the liquid 3. Specifically, in the measurement method according to Preferred Embodiment 4, it is determined whether the liquid 3 is a Newtonian fluid or a non-Newtonian fluid.

To be more specific, the viscosity of a Newtonian fluid is constant regardless of the shear rate. Thus, when the liquid 3 to be measured is a Newtonian fluid, the value of the viscosity calculated from the measurement value of the flow potential is constant regardless of the liquid feeding pressure of the pump 60. On the other hand, the viscosity of a non-Newtonian fluid varies due to the shear rate. Thus, when the liquid 3 to be measured is a non-Newtonian fluid, the value of the viscosity calculated from the measurement value of the flow potential varies depending on the liquid feeding pressure of the pump.

Thus, it is possible to determine whether the liquid 3 is a Newtonian fluid or a non-Newtonian fluid by setting the liquid feeding pressures in suction and discharge to be different from each other and comparing the values of the viscosities calculated from the measurement of the flow potentials in suction and discharge. In addition, since apparent viscosities at a plurality of shear rates are calculated, it is also possible to obtain information about non-Newtonian properties of the measurement target such as a thixotropy index.

An example of the measurement method according to Preferred Embodiment 3 will be described with reference to FIG. 24. Steps ST32 to ST35 and steps ST37 to ST40 that are illustrated in FIG. 24 are the same or substantially the same as steps ST21 to ST28 of Preferred Embodiment 3, and thus, detailed description thereof will be omitted.

As illustrated in FIG. 24, in step ST31, the pump controller 64 sets a liquid feeding pressure of the pump 60 to a first pressure P1. Specifically, the liquid feeding pressure is determined depending on the magnitude of an application voltage to the pump 60. The pump controller 64 adjusts the application voltage to the pump 60 by using the voltage adjuster 65. In Preferred Embodiment 4, the pump controller 64 sets the application voltage to the pump 60 to about +12 V. Due to this, the liquid feeding pressure of the pump 60 is set to the first pressure P1.

Next, by performing steps ST32 to ST35, the first viscosity of the liquid 3 when the liquid 3 is fed (sucked) in the first direction D1 is calculated. Steps ST32 to ST35 are the same as or similar to steps ST21 to ST24 of Preferred Embodiment 3.

In step ST36, the pump controller 64 sets the liquid feeding pressure of the pump 60 to a second pressure P2. A magnitude of the second pressure P2 is different from a magnitude of the first pressure P1. In Preferred Embodiment 4, the pump controller 64 sets the application voltage to the pump 60 to about −24 V. Due to this, the liquid feeding pressure of the pump 60 is set to the second pressure P2.

Next, by performing steps ST37 to ST40, the second viscosity of the liquid 3 when the liquid 3 is fed (discharged) in the second direction D2 is calculated. Steps ST37 to ST40 are the same as or similar to steps ST25 to ST28 of Preferred Embodiment 3.

In step ST41, a characteristic of the liquid 3 is determined by the calculator 50 based on the first viscosity and the second viscosity. Specifically, the calculator 50 compares the first viscosity and the second viscosity. When the first viscosity and the second viscosity are different from each other, the calculator 50 determines that the liquid 3 is a non-Newtonian fluid. When the first viscosity and the second viscosity are equal to each other, the calculator 50 determines that the liquid 3 is a Newtonian fluid.

In this way, in the measurement method according to Preferred Embodiment 4, the characteristic of the liquid 3 can be determined by performing steps ST31 to ST41. Specifically, in the measurement method according to Preferred Embodiment 4, it can be determined whether the liquid 3 is a Newtonian fluid or a non-Newtonian fluid.

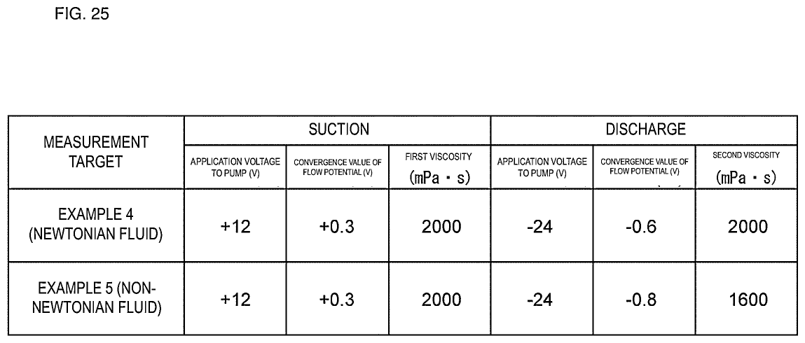

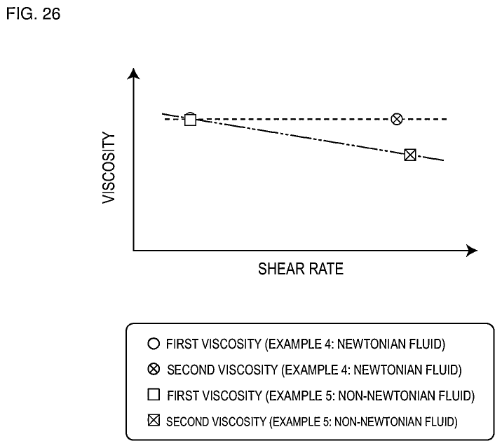

FIG. 25 is a table showing an example of measurement conditions and measurement results in Examples 4 and 5. FIG. 26 is a graph illustrating an example of relationships between viscosities and shear rates in Examples 4 and 5. In Examples 4 and 5 shown in FIG. 25 and illustrated in FIG. 26, the first viscosity of the liquid 3 in suction and the second viscosity of the liquid 3 in discharge were measured by using the fluid characteristic sensor according to Preferred Embodiment 4. In Examples 4 and 5, a Newtonian fluid and a non-Newtonian fluid were used as the liquid 3 to be measured, respectively. Examples 4 and 5 are under the same or substantially the same conditions except for the types of liquid 3. In Examples 4 and 5, the application voltage to the pump 60 in discharge is made larger than the application voltage to the pump 60 in suction. Thus, the second pressure P2 in discharge is made larger than the first pressure P1 in suction.

As shown in FIG. 25 and illustrated in FIG. 26, in Example 4, the first viscosity of the liquid 3 in suction is equal or substantially equal to the second viscosity of the liquid 3 in discharge. On the other hand, in Example 5, the second viscosity of the liquid 3 in discharge is lower than the first viscosity of the liquid 3 in suction. As described above, in the Newtonian fluid of Example 4, the first viscosity and the second viscosity are equal or substantially equal to each other even when the liquid feeding pressures in suction and discharge are different from each other. On the other hand, in the non-Newtonian fluid of Example 5, the first viscosity and the second viscosity are different from each other when the liquid feeding pressure in suction is different from the liquid feeding pressure in discharge. Thus, by comparing the first viscosity and the second viscosity that are measured by using the fluid characteristic sensor according to Preferred Embodiment 4, it is possible to determine whether the liquid 3 to be measured is a Newtonian fluid or a non-Newtonian fluid.

### Advantageous Effects

According to the fluid characteristic sensor of Preferred Embodiment 4, the following advantageous effects can be obtained.

In the fluid characteristic sensor according to Preferred Embodiment 4, the pump controller 64 sets the liquid feeding pressure of the pump 60 when the liquid feeding direction is the first direction D1 to the first pressure P1, and sets the liquid feeding pressure of the pump 60 when the liquid feeding direction is the second direction D2 to the second pressure P2 different from the first pressure P1. With such a configuration, a preferred embodiment of the present invention can be applied to determination of the characteristic of the liquid 3. For example, the calculator 50 can determine whether the liquid 3 is a Newtonian fluid or a non-Newtonian fluid based on the first viscosity and the second viscosity that are measured at different liquid feeding pressures.

Although an example in which the measurement method includes steps ST31 to ST41 has been described in Preferred Embodiment 4, the present invention is not limited thereto. These steps ST31 to ST41 may be divided, integrated, deleted, or added, or the order thereof may be changed.

In Preferred Embodiment 4, an example has been described in which, in step ST41, the liquid 3 is determined to be a Newtonian fluid when the first viscosity and the second viscosity are equal or substantially equal to each other, and the liquid 3 is determined to be a non-Newtonian fluid when the first viscosity and the second viscosity are different from each other, but the present invention is not limited thereto. For example, the calculator 50 may calculate a difference between the first viscosity and the second viscosity, and may determine the characteristic of the liquid 3 based on the difference and a predetermined threshold value. For example, when the difference exceeds the predetermined threshold value, the calculator 50 may determine that the liquid 3 is a non-Newtonian fluid. When the difference does not exceed the predetermined threshold value, the calculator 50 may determine that the liquid 3 is a Newtonian fluid.

### Preferred Embodiment 5

A fluid characteristic sensor and a measurement method according to Preferred Embodiment 5 of the present invention will be described. In Preferred Embodiment 5, differences from Preferred Embodiment 2 will be mainly described. In Preferred Embodiment 5, elements that are the same as or equivalent to those in Preferred Embodiment 2 are denoted by the same reference signs. Further, in Preferred Embodiment 5, description overlapping with that of Preferred Embodiment 2 will be omitted.

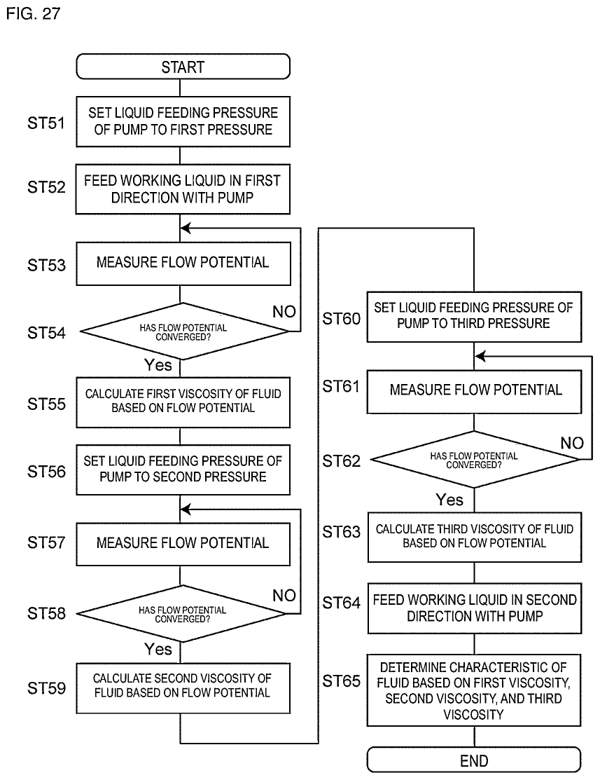

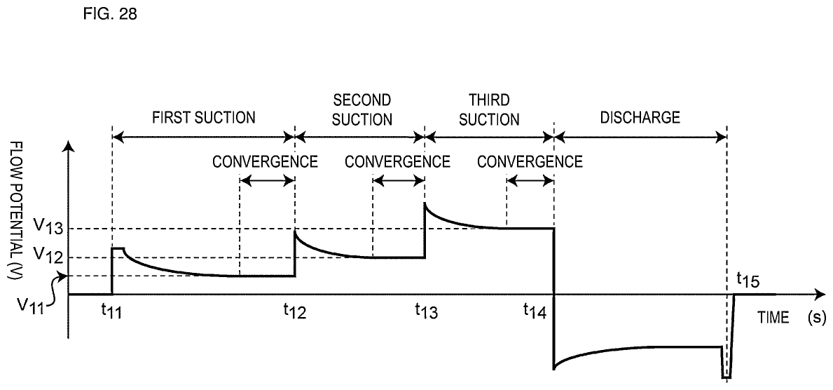

An example of a measurement method using the fluid characteristic sensor according to Preferred Embodiment 5 will be described with reference to FIG. 27 and FIG. 28. FIG. 27 is a flowchart of the example of the measurement method according to Preferred Embodiment 5 of the present invention. FIG. 28 is a graph illustrating an example of a change in flow potential measured by the fluid characteristic sensor according to Preferred Embodiment 5 of the present invention.

Preferred Embodiment 5 is different from Preferred Embodiment 2 in that a viscosity is measured while a liquid feeding pressure of the liquid 3 is changed in a stepwise manner.

In the measurement method according to Preferred Embodiment 5, the characteristic of the liquid 3 is determined based on a plurality of viscosities measured by changing the liquid feeding pressure in the stepwise manner in suction and/or discharge of the liquid 3 to be measured. To be specific, in the measurement method according to Preferred Embodiment 5, it is determined which type of the liquid 3 is used among a Newtonian fluid, a pseudoplastic fluid, and a Bingham fluid.

More specifically, by measuring the viscosity of the liquid 3 a plurality of times while changing the liquid feeding pressure of the liquid 3, it is possible to obtain data of the viscosity that changes in relation to the liquid feeding pressure. The change tendency of a viscosity differs depending on a fluid. For example, in the case of a Newtonian fluid, the viscosity does not change even when the liquid feeding pressure changes. In the case of a pseudoplastic fluid, the viscosity decreases in proportion to the liquid feeding pressure. In the case of a Bingham fluid, the flow rate rapidly decreases as the liquid feeding pressure increases, but becomes constant when the flow rate exceeds a predetermined liquid feeding pressure.

Thus, it is possible to determine the characteristic of the liquid 3 to be measured based on the tendency of a change in viscosity measured by changing the liquid feeding pressure in a stepwise manner.

An example of the measurement method according to Preferred Embodiment 5 will be described with reference to FIG. 27 and FIG. 28. Steps ST52 to ST55, ST57 to ST59, and ST61 to ST63 illustrated in FIG. 27 are the same or substantially the same as steps ST11 to ST14 of Preferred embodiment 2, and thus, detailed description thereof will be omitted.

As illustrated in FIG. 27, in step ST51, the pump controller 64 sets the liquid feeding pressure of the pump 60 to the first pressure P1. Specifically, the liquid feeding pressure is determined depending on the magnitude of an application voltage to the pump 60. The pump controller 64 adjusts the application voltage to the pump 60 by using the voltage adjuster 65. In Preferred Embodiment 4, the pump controller 64 sets the application voltage to the pump 60 in the first suction to about +12 V. Due to this, the liquid feeding pressure in the first suction of the pump 60 is set to the first pressure P1.

Next, the first viscosity of the liquid 3 in the first suction is calculated by performing steps ST52 to ST55. Steps ST52 to ST54 are the same as or similar to steps ST11 to ST14 of Preferred Embodiment 2. To be specific, the first suction of the liquid 3 is performed by the pump 60 feeding the working liquid 4 in the first direction D1 at the first pressure P1. As illustrated in FIG. 28, when the first suction is started at a time t₁₁, the flow potential increases. Thereafter, the flow potential decreases with the lapse of time. The calculator 50 calculates the first viscosity of the liquid 3 by using a convergence value V₁₁ when the flow potential converges in the first suction.

In step ST56, the pump controller 64 sets the liquid feeding pressure of the pump 60 to the second pressure P2. The second pressure P2 is different from the first pressure P1. In Preferred Embodiment 4, the second pressure P2 is set to be larger than the first pressure P1. For example, the pump controller 64 sets the application voltage to the pump 60 in the second suction to about +18 V. Due to this, the liquid feeding pressure in the second suction of the pump 60 is set to the second pressure P2.

Next, the second viscosity of the liquid 3 in the second suction is calculated by performing steps ST57 to ST59. Steps ST57 to ST59 are the same as or similar to steps ST12 to ST14 of Preferred Embodiment 2. To be specific, the second suction of the liquid 3 is performed by the pump 60 feeding the working liquid 4 in the first direction D1 at the second pressure P2. As illustrated in FIG. 28, when the second suction is started at a time t₁₂, the flow potential increases. Thereafter, the flow potential decreases with the lapse of time. The calculator 50 calculates the second viscosity of the liquid 3 by using the convergence value V₁₂ when the flow potential converges in the second suction.

In step ST60, the pump controller 64 sets the liquid feeding pressure of the pump 60 to a third pressure P3. The third pressure P3 is different from the first pressure P1 and the second pressure P2. In Preferred Embodiment 4, the third pressure P3 is set to be larger than the second pressure P2. For example, the pump controller 64 sets the application voltage to the pump 60 in a third suction to about +24 V. Thus, the liquid feeding pressure in the third suction of the pump 60 is set to the third pressure P3.

Next, a third viscosity of the liquid 3 in the third suction is calculated by performing steps ST61 to ST63. Steps ST61 to ST63 are the same as or similar to steps ST12 to ST14 of Preferred Embodiment 2. To be specific, the third suction of the liquid 3 is performed by the pump 60 feeding the working liquid 4 in the first direction D1 at the third pressure P3. As illustrated in FIG. 28, when the third suction is started at a time t₁₃, the flow potential increases. Thereafter, the flow potential decreases with the lapse of time. The calculator 50 calculates the third viscosity of the liquid 3 by using the convergence value V₁₃ when the flow potential converges in the third suction.

In step ST64, the working liquid 4 is fed in the second direction D2 by the pump 60. Thus, the liquid 3 is discharged.

In step ST65, the characteristic of the liquid 3 is determined by the calculator 50 based on the first viscosity, the second viscosity, and the third viscosity. Specifically, based on the first viscosity, the second viscosity, and the third viscosity, the calculator 50 calculates a change tendency in the viscosity of the liquid 3 accompanying a change in the liquid feeding pressure. The calculator 50 determines the characteristic of the liquid 3 based on the change tendency in the viscosity of the liquid 3 accompanying the change in the liquid feeding pressure. For example, the calculator 50 determines which type of the liquid 3 is used among a Newtonian fluid, a pseudoplastic fluid, and a Bingham fluid, based on the change tendency in the viscosity of the liquid 3 accompanying the change in the liquid feeding pressure.

Thus, in the measurement method according to Preferred Embodiment 5, the characteristic of the liquid 3 can be determined by performing steps ST51 to ST65. To be specific, in the measurement method according to Preferred Embodiment 5, it can be determined which type of the liquid 3 is used among a Newtonian fluid, a pseudoplastic fluid, and a Bingham fluid.

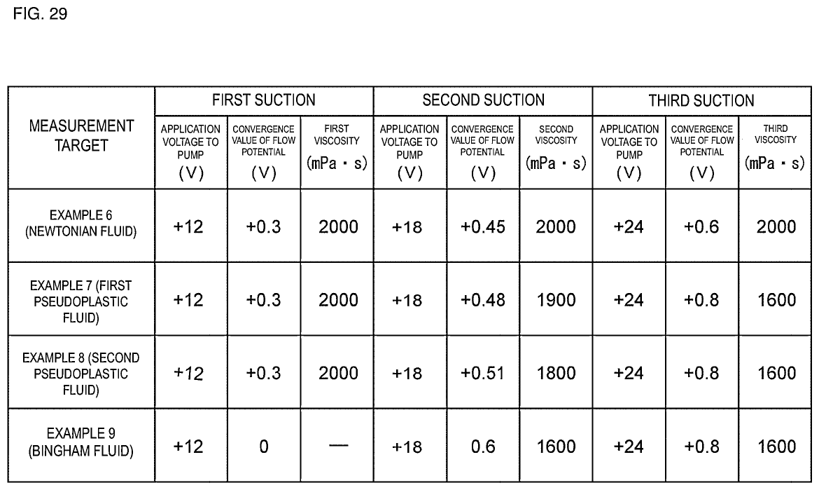

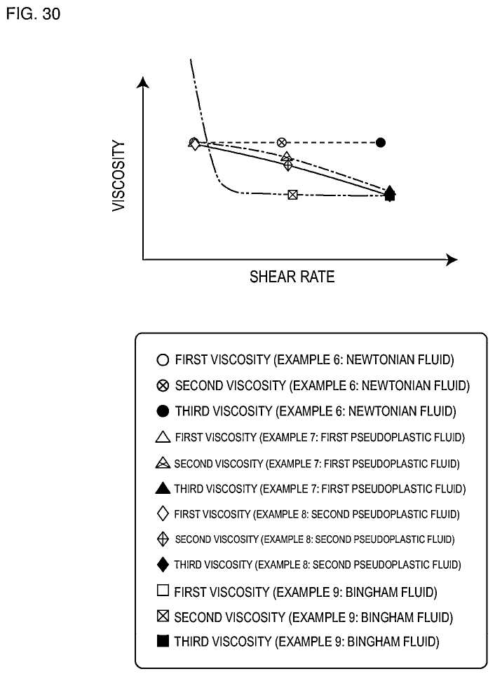

FIG. 29 is a table showing an example of measurement conditions and measurement results of Examples 6 to 9. FIG. 30 is a graph illustrating an example of relationships between viscosities and shear rates in Examples 1 and 3. In Examples 6 to 9 shown in FIG. 29 and illustrated in FIG. 30, the first viscosity of the liquid 3 in the first suction, the second viscosity of the liquid 3 in the second suction, and the third viscosity of the liquid 3 in the third suction were measured by using the fluid characteristic sensor according to Preferred Embodiment 5. In Examples 6 to 9, a Newtonian fluid, a first pseudoplastic fluid, a second pseudoplastic fluid, and a Bingham fluid were used as the liquid 3 to be measured, respectively. Examples 6 to 9 have the same conditions except for the types of the liquid 3. In FIG. 30, a shear rate on the horizontal axis is proportional to a flow potential (flow rate of the liquid 3).

As shown in FIG. 29 and illustrated in FIG. 30, in Example 6, the viscosity of the liquid 3 does not change regardless of the change in the shear rate (liquid feeding pressure), and is a constant value. In Examples 7 and 8, the viscosity of the liquid 3 gradually decreases as the shear rate (liquid feeding pressure) increases. In Example 9, the viscosity of the liquid 3 rapidly decreases as the shear rate (liquid feeding pressure) increases, and then becomes a constant value. As described above, in Examples 6 to 9, the change tendencies in the viscosities accompanying the change in the liquid feeding pressure are different from each other. Thus, it is possible to determine which type of the liquid 3 to be measured is used among a Newtonian fluid, a first pseudoplastic fluid, a second pseudoplastic fluid, or a Bingham fluid, based on the change tendency in the viscosity accompanying the change in the liquid feeding pressure.

### Advantageous Effects

According to the fluid characteristic sensor of Preferred Embodiment 5, the following advantageous effects can be obtained.

In the fluid characteristic sensor according to Preferred Embodiment 5, the pump controller 64 changes the liquid feeding pressure in the stepwise manner. With such a configuration, a preferred embodiment the present invention can be applied to determination of the characteristic of the liquid 3. For example, the calculator 50 can determine which type of the liquid 3 is used among a Newtonian fluid, a first pseudoplastic fluid, a second pseudoplastic fluid, and a Bingham fluid based on information about a plurality of viscosities measured at different liquid feeding pressures.

Although an example in which the pump controller 64 changes the liquid feeding pressure at the three steps has been described in Preferred Embodiment 5, the present invention is not limited thereto. The pump controller 64 may change the liquid feeding pressure at two or more steps.

In Preferred Embodiment 5, an example in which the pump controller 64 changes the liquid feeding pressure in sucking the liquid 3 in the stepwise manner has been described, but the present invention is not limited thereto. For example, the pump controller 64 may change the liquid feeding pressure in discharging the liquid 3 in a stepwise manner.

Hereinafter, comparative examples will be described for reference.

### COMPARATIVE EXAMPLE 1

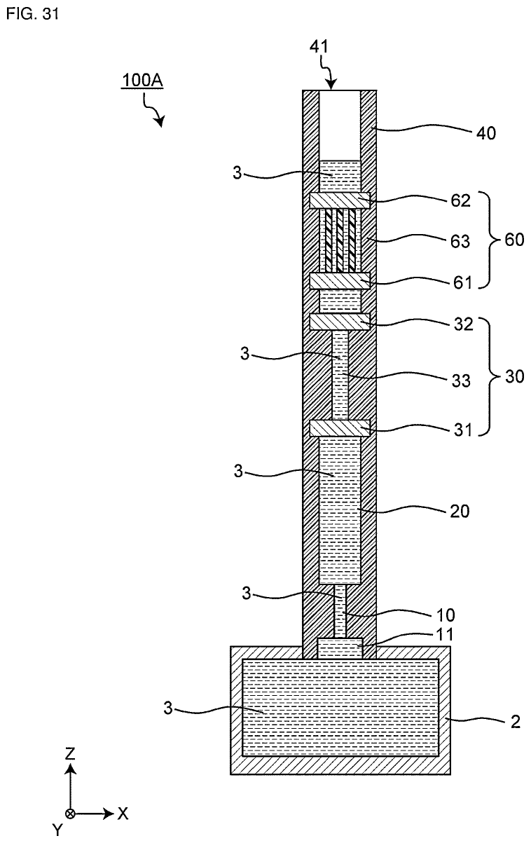

FIG. 31 is a schematic diagram of a fluid characteristic sensor 100A according to Comparative Example 1. As illustrated in FIG. 31, the fluid characteristic sensor 100A according to Comparative Example 1 has a similar configuration to that of the fluid characteristic sensor 1B according to Preferred Embodiment 2, except that the movable partition wall 21 and the working liquid 4 are not provided. That is, in Comparative Example 1, a flow potential of the liquid 3 to be measured is measured.

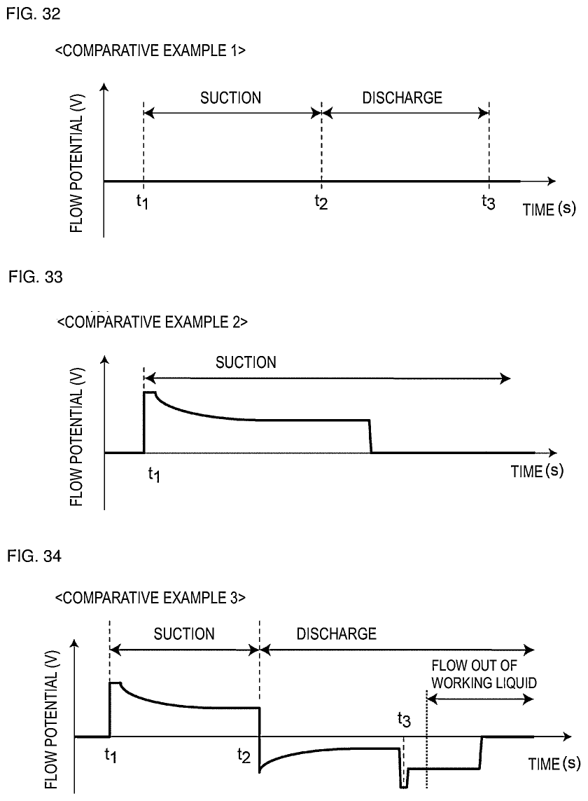

FIG. 32 is a graph illustrating an example of a change in flow potential measured by the fluid characteristic sensor 100A according to Comparative Example 1. FIG. 32 illustrates an example of a change in flow potential when the liquid 3 to be measured is a non-polar solvent. As illustrated in FIG. 32, when the flow potential of the non-polar solvent was measured by the fluid characteristic sensor 100A according to Comparative Example 1, the flow potential could not be measured. As described above, in Comparative Example 1, it is not possible to measure a liquid such as a non-polar solvent where a flow potential is hardly generated. On the other hand, the fluid characteristic sensor 1B according to Preferred Embodiment 2 of the present invention has a configuration in which the liquid 3 to be measured and the working liquid 4 that is a polar solvent are separated from each other by the movable partition wall 21. Thus, the flow potential generated by the flow of the working liquid 4 can be measured, and the liquid 3 such as a non-polar solvent can also be measured.

### COMPARATIVE EXAMPLE 2

In Comparative Example 2, a similar configuration to that of the fluid characteristic sensor 1B according to Preferred Embodiment 2, except that the pump cannot change the liquid feeding direction. FIG. 33 is a graph illustrating an example of a change in flow potential measured by a fluid characteristic sensor according to Comparative Example 2. As illustrated in FIG. 33, in Comparative Example 2 in which the liquid 3 to be measured cannot be discharged, the liquid 3 is continuously sucked. When the liquid 3 is continuously sucked, the movable partition wall 21 defined by a gas and the liquid 3 enter the potential measurer 30, so that the flow potential becomes 0. Because of this, the measurement cannot be performed. On the other hand, in the fluid characteristic sensor 1B according to Preferred Embodiment 2 of the present invention, since the pump 60 capable of changing the liquid feeding direction is used, the liquid 3 can be sucked and discharged. This makes it possible to continuously measure a characteristic of the liquid 3.

### COMPARATIVE EXAMPLE 3

In Comparative Example 3, a similar configuration to that of the fluid characteristic sensor 1B according to Preferred Embodiment 2, except that control for stopping the pump 60 based on the flow potential is not performed. FIG. 34 is a graph illustrating an example of a change in flow potential measured by a fluid characteristic sensor according to Comparative Example 3. As illustrated in FIG. 34, in Comparative Example 3 in which the control for stopping the pump 60 based on the flow potential is not performed, the liquid 3 is continuously discharged. Thus, the movable partition wall 21 defined by a gas and the working liquid 4 flow out to the outside of the fluid characteristic sensor through the pressure loss generator 10. Thus, the working liquid 4 is mixed into the liquid 3 in the container 2. On the other hand, in the fluid characteristic sensor 1B according to Preferred Embodiment 2 of the present invention, the control to stop the pump 60 is performed based on the flow potential, so that the flow out of the working liquid 4 to the outside of the fluid characteristic sensor 1B can be reduced or prevented.

### Preferred Embodiment 6

A fluid characteristic sensor according to Preferred Embodiment 6 of the present invention will be described. In Preferred Embodiment 6, differences from Preferred Embodiment 2 will be mainly described. In Preferred Embodiment 6, elements that are the same as or equivalent to those in Preferred Embodiment 2 are denoted by the same reference signs. Further, in Preferred Embodiment 6, description overlapping with that of Preferred Embodiment 2 will be omitted.

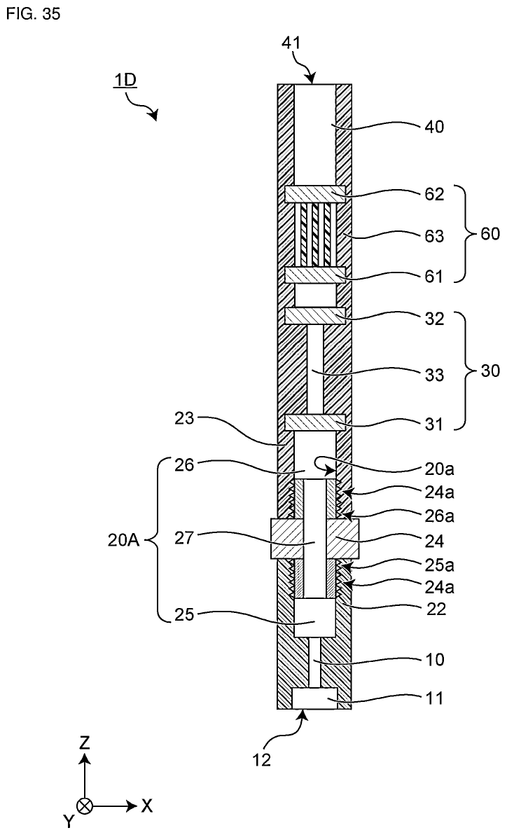

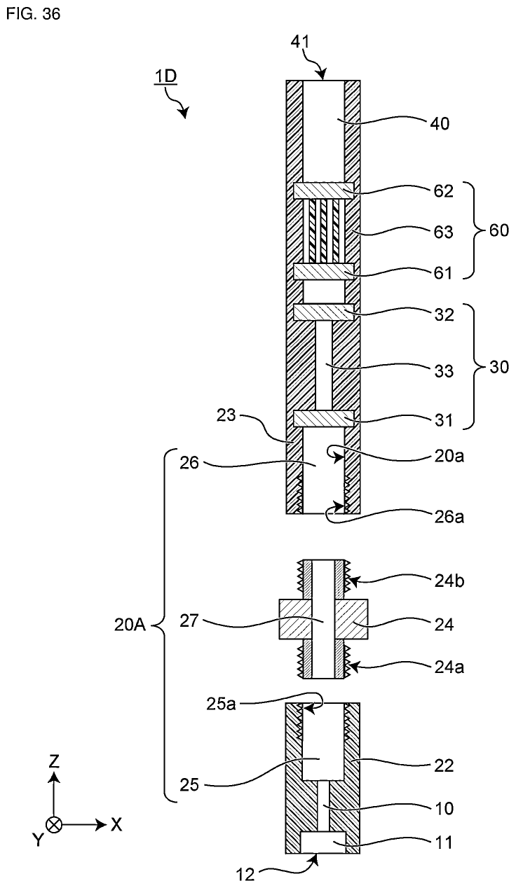

An example of the fluid characteristic sensor according to Preferred Embodiment 6 will be described with reference to FIG. 35 and FIG. 36. FIG. 35 is a schematic configuration diagram of the example of the fluid characteristic sensor according to Preferred Embodiment 6 of the present invention. FIG. 36 is a schematic exploded diagram of the fluid characteristic sensor illustrated in FIG. 35. In FIG. 35 and FIG. 36, the working liquid 4 is not illustrated.

Preferred Embodiment 6 is different from Preferred Embodiment 2 in that an attachment portion 22 including the inflow/outflow port 12 and the pressure loss generator 10 is detachably attached to a main body 23 including at least a portion of a first flow path 20A.

As illustrated in FIG. 35 and FIG. 36, a fluid characteristic sensor 1D according to Preferred Embodiment 6 further includes the attachment portion 22, the main body 23, and a connector 24. The connector 24 is not necessarily provided.

The attachment portion 22 includes the inflow/outflow port 12 through which the liquid 3 as a fluid flows in and out and the pressure loss generator 10. The attachment portion 22 is detachably attached to the main body 23. The attachment portion 22 has, for example, a tube shape including one end and another end. The attachment portion 22 has, for example, a cylindrical or substantially cylindrical shape. The attachment portion 22 may be defined by, for example, a pipe.

In Preferred Embodiment 6, the attachment portion 22 includes the inflow/outflow port 12, the fluid flow path 11, the pressure loss generator 10, and a first connection flow path 25. The inflow/outflow port 12 is provided at the one end of the attachment portion 22, and an opening of the first connection flow path 25 is provided at the other end of the attachment portion 22.

The first connection flow path 25 is connected to the pressure loss generator 10 and defines a portion of the first flow path 20A. The first connection flow path 25 has, for example, a cylindrical or substantially cylindrical shape. A flow path cross-sectional area of the first connection flow path 25 is larger than a flow path cross-sectional area of the pressure loss generator 10.

A first female thread portion 25a is provided at an inner wall of the first connection flow path 25. The first female thread portion 25a is screwed with the first male thread portion 24a of the connector 24.

The main body 23 includes at least a portion of the first flow path 20A. The attachment portion 22 is detachably attached to the main body 23. The main body 23 has, for example, a tube shape including one end and another end. The main body 23 has, for example, a cylindrical or substantially cylindrical shape. The main body 23 may be defined by a pipe, for example.

In Preferred Embodiment 6, the main body 23 includes a second connection flow path 26, the potential measurer 30, the working liquid flow path 40, and the pump 60. An opening of the second connection flow path 26 is provided at the one end of the main body 23, and an opening of the working liquid flow path 40 is provided at the other end (the open end 41) of the main body 23.

The second connection flow path 26 is connected to the potential measurer 30, and defines a portion of the first flow path 20A. The second connection flow path 26 has, for example, a cylindrical or substantially cylindrical shape. A flow path cross-sectional area of the second connection flow path 26 is larger than a flow path cross-sectional area of the pressure loss generator 10. For example, the flow path cross-sectional area of the second connection flow path 26 is equal or substantially equal to the flow path cross-sectional area of the first connection flow path 25.

A second female thread portion 26a is provided at an inner wall of the second connection flow path 26. The second female thread portion 26a is screwed with a second male thread portion 24b of the connector 24.

In Preferred Embodiment 6, the attachment portion 22 is attached to the main body 23 by using the connector 24. The connector 24 is a cylindrical or substantially cylindrical member including one end and the other end. The first male thread portion 24a and the second male thread portion 24b are provided at outer walls at one end side and the other end side of the connector 24, respectively. Further, the connector 24 includes a third connection flow path 27. The connector 24 is, for example, a nipple.

A flow path cross-sectional area of the third connection flow path 27 is larger than a flow path cross-sectional area of the pressure loss generator 10. The third connection flow path 27 defines a portion of the first flow path 20A. To be more specific, when the attachment portion 22 and the main body 23 are attached to each other with the connector 24 interposed therebetween, the first connection flow path 25, the second connection flow path 26, and the third connection flow path 27 communicate with each other to form the first flow path 20A.

In other words, the first flow path 20A is configured to be separable into a plurality of flow paths. To be specific, the first flow path 20A is configured to be separable into the first connection flow path 25, the second connection flow path 26, and the third connection flow path 27.

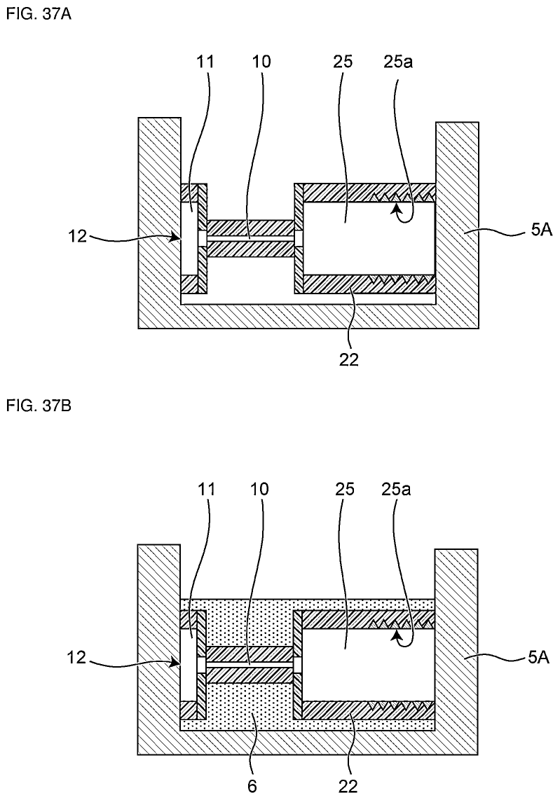

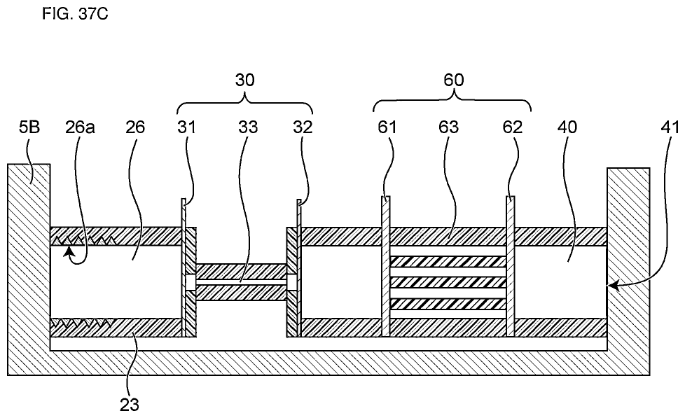

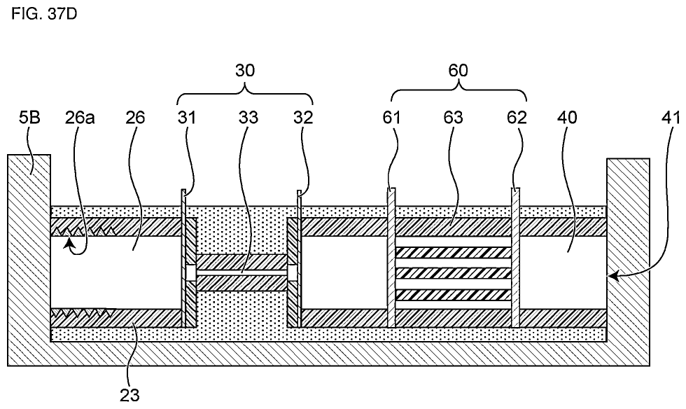

A non-limiting example of a manufacturing method of the fluid characteristic sensor 1D will be described with reference to FIGS. 37A to 37D. FIGS. 37A to 37D are schematic views illustrating an example of a manufacturing process of the fluid characteristic sensor 1D according to Preferred Embodiment 6 of the present invention. FIGS. 37A and 37B illustrate an example of a manufacturing process of the attachment portion 22, and FIGS. 37C and 37D illustrate an example of a manufacturing process of the main body 23.

As illustrated in FIG. 37A, elements of the attachment portion 22 are disposed in a mold 5A. The mold 5A is formed in a recessed shape. To be specific, the fluid flow path 11, the pressure loss generator 10, and the first connection flow path 25 are disposed in the mold 5A in a state of being connected to each other. The first connection flow path 25 is, for example, a resin pipe provided with the first female thread portion 25a at the inner wall thereof.

At this time, the inflow/outflow port 12 of the fluid flow path 11 and the opening of the first connection flow path 25 are pressed against an inner wall of the mold 5A. That is, the inflow/outflow port 12 and the opening of the first connection flow path 25 are sealed by the inner wall of the mold 5A. When the elements of the attachment portion 22 are disposed in the mold 5A, these elements may be bonded with, for example, an adhesive or the like.

As illustrated in FIG. 37B, the molten sealing material 6 is introduced into the mold 5A in which the elements of the attachment portion 22 are disposed, and is cured. After the sealing material 6 is cured, the mold 5A is removed to obtain the attachment portion 22.

As illustrated in FIG. 37C, elements of the main body 23 are disposed in a mold 5B. The mold 5B is formed in a recessed shape. To be specific, the second connection flow path 26, the potential measurer 30, the working liquid flow path 40, and the pump 60 are disposed in the mold 5B in a state of being connected to each other. The second connection flow path 26 is, for example, a resin pipe provided with the second female thread portion 26a at the inner wall thereof.

At this time, an opening of the second connection flow path 26 and the open end 41 of the working liquid flow path 40 are pressed against the inner wall of the mold 5B. That is, the opening of the second connection flow path 26 and the open end 41 of the working liquid flow path 40 are sealed by the inner wall of the mold 5B. When the elements of the main body 23 are disposed in the mold 5B, these elements may be bonded by using, for example, an adhesive or the like.

As illustrated in FIG. 37D, the molten sealing material 6 is introduced into the mold 5B in which the elements of the main body 23 are disposed, and is cured. After the sealing material 6 is cured, the mold 5B is removed to obtain the main body 23. Thereafter, the working liquid 4 is put into the inside of the main body 23.

In this way, the fluid characteristic sensor 1D can be manufactured.

### Advantageous Effects

According to the fluid characteristic sensor 1D according to Preferred Embodiment 6, the following advantageous effects can be obtained.

The fluid characteristic sensor 1D includes the attachment portion 22 including the inflow/outflow port 12 through which the fluid flows in/out and the pressure loss generator 10, and the main body 23 that includes at least a portion of the first flow path 20A and to which the attachment portion 22 is detachably attached.

With such a configuration, the attachment portion 22 including the inflow/outflow port 12 can be easily attached to and detached from the main body 23. That is, in the fluid characteristic sensor 1D, the attachment portion 22 including the inflow/outflow port 12 and the pressure loss generator 10 is replaceable. Thus, when a measurement target is changed, the measurement can be easily performed by replacing the attachment portion 22, and usability for a user is improved.

In addition, when the measurement target is changed, it is possible to reduce or prevent mixing of the different measurement targets.

In addition, since the fluid characteristic sensor 1D can be used for a different measurement target by replacing the attachment portion 22, it is not necessary to clean the fluid characteristic sensor 1D.

In addition, since the attachment portion 22 can be easily replaced, it is possible to shorten a period of time until the measurement target is changed and the next measurement is performed.

Further, the attachment portion 22 can be replaced with an attachment portion 22 including an optimum pressure loss generator 10 depending on a characteristic of a fluid to be measured, which can improve the measurement accuracy. For example, highly accurate viscosity measurement can be performed over a wide viscosity range by replacing the attachment portion 22 with an attachment portion 22 including a pressure loss generator 10 with an optimum flow path diameter depending on the viscosity of a fluid.

As the flow path diameter of the pressure loss generator 10 decreases, a pressure loss generated in the pressure loss generator 10 becomes larger than a pressure loss generated in a peripheral flow path and the potential measurer 30, and thus, a variation amount in flow rate accompanying a change in viscosity of a measurement target increases. Thus, the measurement accuracy is improved. On the other hand, since the obtained flow rate decreases as the flow path diameter decreases, only a minute flow rate is generated in a case of a high viscosity, and the measurement accuracy of the flow potential may decrease. Thus, highly accurate viscosity measurement can be performed by replacing the attachment portion 22 with an attachment portion 22 with a pressure loss generator 10 having a suitable flow path diameter according to a viscosity range to be measured.

In Preferred Embodiment 6, an example in which the attachment portion 22 and the main body 23 are attached by using the connector 24 has been described, but the present invention is not limited thereto. The connector 24 is not necessarily provided. For example, the attachment portion 22 may be directly attached to the main body 23. In this case, a male thread portion may be provided at the outer wall of the attachment portion 22.

In Preferred Embodiment 6, an example in which the first connection flow path 25 defines a portion of the first flow path 20A has been described, but the present invention is not limited thereto. The first connection flow path 25 does not need to define a portion of the first flow path 20A. In this case, the first connection flow path 25 may be used as a portion connected to the main body 23.

In Preferred Embodiment 6, an example in which the attachment portion 22, the main body 23, and the connector 24 are connected to each other by using the threads has been described, but the present invention is not limited thereto. The attachment portion 22, the main body 23, and the connector 24 may be connected by a mechanism other than the threads.

### Preferred Embodiment 7

A fluid characteristic sensor according to Preferred Embodiment 7 of the present invention will be described. In Preferred Embodiment 7, differences from Preferred Embodiment 2 will be mainly described. In Preferred Embodiment 7, elements that are the same as or equivalent to those in Preferred Embodiment 2 are denoted by the same reference signs. Further, in Preferred Embodiment 7, description overlapping with that of Preferred Embodiment 2 will be omitted.

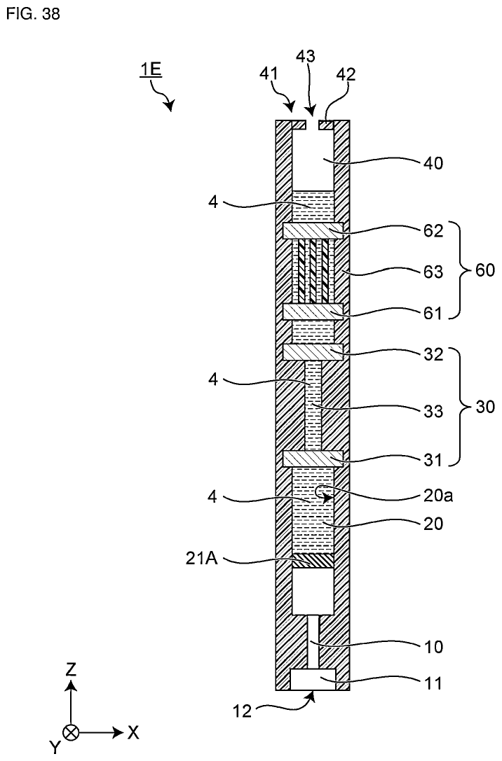

An example of the fluid characteristic sensor according to Preferred Embodiment 7 will be described with reference to FIG. 38. FIG. 38 is a schematic configuration diagram of the example of the fluid characteristic sensor according to Preferred Embodiment 7 of the present invention.

Preferred Embodiment 7 differs from Preferred Embodiment 2 in that a partition wall 21A is defined by or includes a solid.

As illustrated in FIG. 38, in a fluid characteristic sensor 1E according to Preferred Embodiment 7, the partition wall 21A is defined by or includes a solid. For example, the partition wall 21A is made of or includes rubber, plastic, or the like. Examples of the rubber include fluororubber, chloroprene rubber, nitrile rubber, ethylene propylene diene rubber, and silicone rubber. Examples of the plastic include polytetrafluoroethylene, polyethylene, polypropylene, a cycloolefin polymer, and a cyclic olefin-based copolymer. Hereinafter, the “partition wall 21A” may be referred to as a “solid partition wall 21A”.

The solid partition wall 21A is made of or includes a plate-shaped structure. For example, the solid partition wall 21A has a disc shape. The solid partition wall 21A is movable in the first flow path 20. To be specific, the solid partition wall 21A moves while being in contact with the inner wall 20a of the first flow path 20.

An outside diameter of the solid partition wall 21A is, for example, equal or substantially equal to the flow path diameter of the first flow path 20. For example, the outside diameter of the solid partition wall 21A may be larger than the flow path diameter of the first flow path 20 by about 5% or less. With such a configuration, the solid partition wall 21A can move in the first flow path 20 while securing sealing performance.

Additionally, in the fluid characteristic sensor 1F, a cover 42 is disposed at the open end 41A of the working liquid flow path 40. The cover 42 is provided with a through hole 43. For example, the through hole 43 has a diameter of about 1 mm. In this way, by disposing the cover 42 provided with the through hole 43 at the open end 41A, leakage of the working liquid 4 from the open end 41A can be reduced or prevented when the fluid characteristic sensor 1F is inclined or the like.

### Advantageous Effects

According to the fluid characteristic sensor 1E according to Preferred Embodiment 7, the following advantageous effects can be obtained.

In the fluid characteristic sensor 1E, the partition wall 21A is defined by or includes a solid.

With such a configuration, the fluid to be measured and the working liquid 4 can be easily separated from each other. In addition, even when vibration or inclination is applied to the fluid characteristic sensor 1E, the fluid and the working liquid 4 can be more reliably partitioned from each other, and thus, mixture of the fluid to be measured into the working liquid 4 can be reduced or prevented.

In addition, when the partition wall 21A is a solid, it is easy to secure the sealing performance between the partition wall 21A and the inner wall 20a of the first flow path 20, compared to a partition wall that is defined by or includes a gas. Thus, it is possible to improve the flexibility of an installation place and an installation form of the fluid characteristic sensor 1E.

For example, the fluid characteristic sensor 1E can be installed in a gravity direction, a horizontal direction, or a direction oblique to these directions.

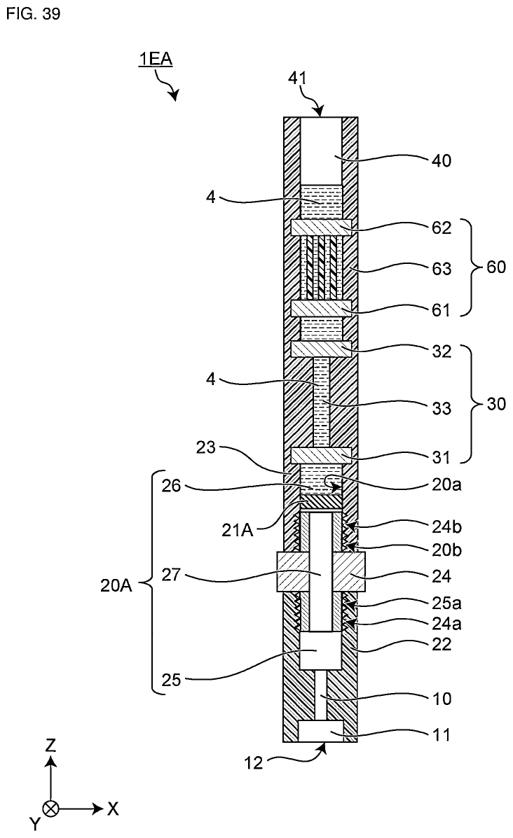

FIG. 39 is a schematic configuration diagram of an example of a fluid characteristic sensor according to Modification 5 of Preferred Embodiment 7 of the present invention. As illustrated in FIG. 39, a fluid characteristic sensor 1EA of Modification 5 is provided by combining the configuration of the fluid characteristic sensor 1D according to Preferred Embodiment 6 with the solid partition wall 21A according to Preferred Embodiment 7.

In the fluid characteristic sensor 1EA, the solid partition wall 21A is disposed in the second connection flow path 26 defining a portion of the first flow path 20A. That is, in the fluid characteristic sensor 1EA, the solid partition wall 21A may be disposed in the first flow path 20A at the main body 23 side.

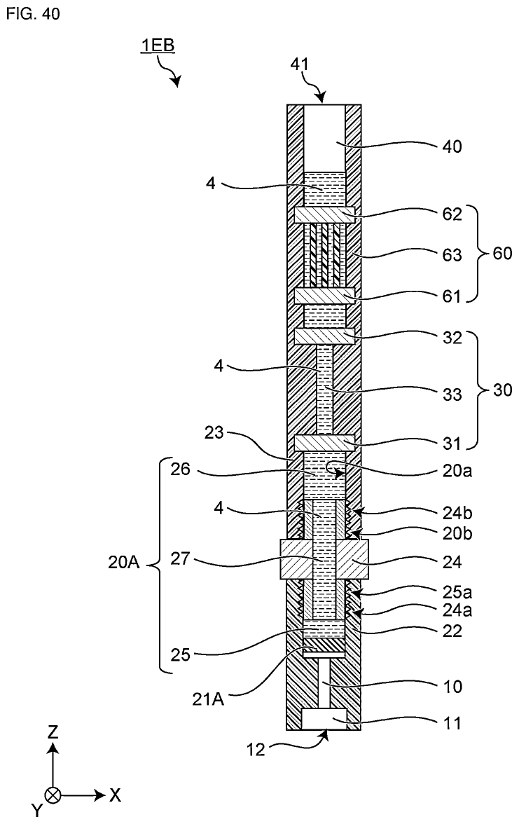

FIG. 40 is a schematic configuration diagram of an example of a fluid characteristic sensor according to Modification 6 of Preferred Embodiment 7 of the present invention. As illustrated in FIG. 40, a fluid characteristic sensor 1EB according to Modification 6 is provided by combining the configuration of the fluid characteristic sensor 1D according to Preferred Embodiment 6 with the solid partition wall 21A according to Preferred Embodiment 7.

In the fluid characteristic sensor 1EB, the solid partition wall 21A is disposed in the first connection flow path 25 defining a portion of the first flow path 20A. That is, in the fluid characteristic sensor 1EB, the solid partition wall 21A may be disposed in the first flow path 20A at the attachment portion 22 side.

Next, another example of the solid partition wall will be described.

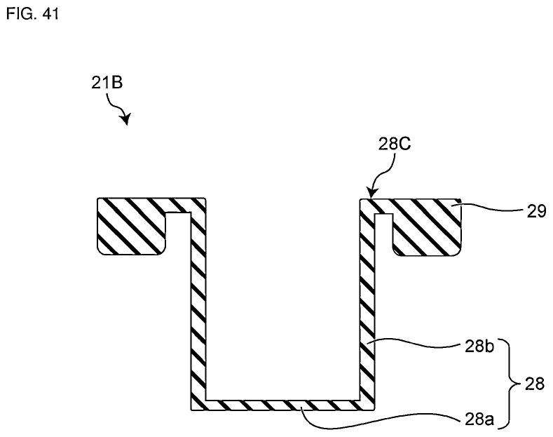

FIG. 41 is a schematic diagram illustrating another example of the solid partition wall. As illustrated in FIG. 41, a solid partition wall 21B includes a partition wall main body 28 that has a recessed shape and that is elastically deformable, and a flange 29 that protrudes outward from an outer wall of the partition wall main body 28.

The partition wall main body 28 has a bottomed tube shape. Specifically, the partition wall main body 28 includes a bottom portion 28a and a side wall 28b. The bottom portion 28a has a disc shape. The side wall 28b has a cylindrical or substantially cylindrical shape extending from an outer periphery of the bottom portion 28a in a thickness direction of the bottom portion 28a. The side wall 28b includes one end and another end. At the one end of the side wall 28b, the bottom portion 28a is disposed. The other end of the side wall 28b is an end portion opposite to the one end, and is opened. That is, the other end of the side wall 28b defines an open end 28c.

The partition wall main body 28 is elastically deformable by receiving an external force.

The flange 29 protrudes radially outward from the side wall 28b of the partition wall main body 28. The flange 29 is provided at the other end of the side wall of the partition wall main body 28. The flange 29 has a ring shape. The flange 29 is used as a portion to grip the solid partition wall 21B.

The solid partition wall 21B is made of, for example, rubber. Examples of the rubber material include ethylene propylene diene rubber (EPDM), acrylonitrile butadiene rubber (NBR), and fluorine rubber (FKM).

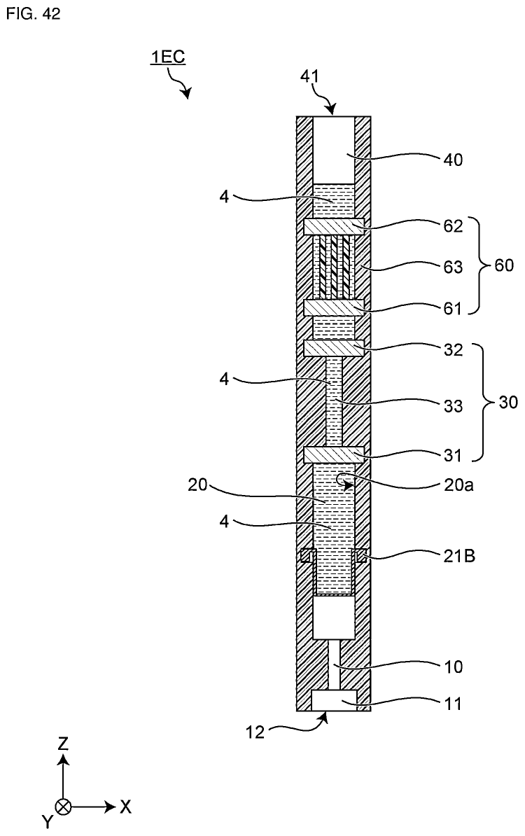

FIG. 42 is a schematic configuration diagram of an example of a fluid characteristic sensor according to Modification 7 of Preferred Embodiment 7 of the present invention. As illustrated in FIG. 42, a fluid characteristic sensor 1EC according to Modification 7 includes the solid partition wall 21B illustrated in FIG. 41.

The solid partition wall 21B is fixed in the first flow path 20. To be more specific, the flange 29 of the solid partition wall 21B is held to fix the solid partition wall 21B. The bottom portion 28a of the solid partition wall 21B is disposed in a direction intersecting the inner wall 20a of the first flow path 20, and the side wall 28b is disposed along the inner wall 20a of the first flow path 20.

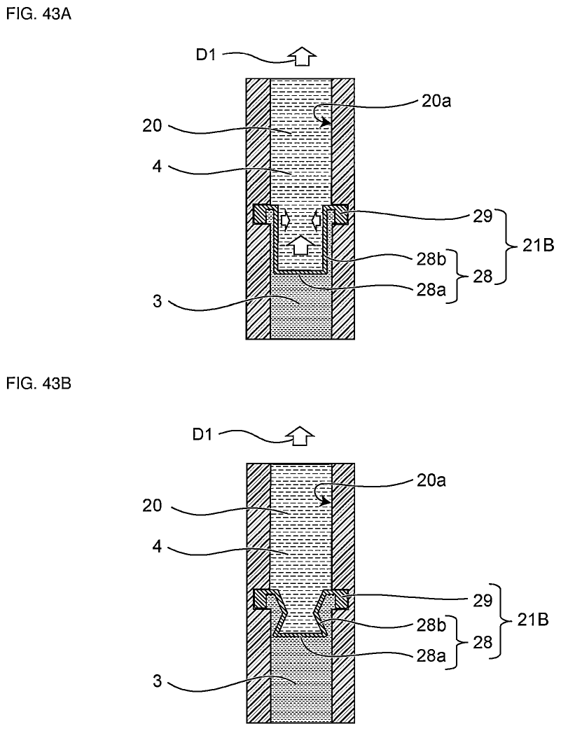

FIGS. 43A and 43B are schematic diagrams for describing an example of an operation of the solid partition wall. FIGS. 43A and 43B illustrate an example of an operation of sucking the liquid 3 to be measured in the first direction D1. As illustrated in FIGS. 43A and 43B, when the liquid 3 is sucked in the first direction D1, the solid partition wall 21B is elastically deformed. Specifically, the suction of the liquid 3 in the first direction D1 generates a force directed inward with respect to the partition wall main body 28 of the solid partition wall 21B. Because of this, the side wall 28b of the partition wall main body 28 is elastically deformed so as to be recessed radially inward, and the bottom portion 28a of the partition wall main body 28 moves in the first direction D1.

The solid partition wall 21B is elastically deformed in this manner, which leads to the flow of the working liquid 4 in the first direction D1. Thus, the potential measurer 30 can measure the flow potential generated when the working liquid 4 flows.

Further, in order to measure the viscosity, the liquid 3 is sucked until the pressure loss generator 10 is filled with the liquid 3. Thus, it is preferable that the volume of the space surrounded by the partition wall main body 28 of the solid partition wall 21B changes to some extent. In the case of the solid partition wall 21B, when the working liquid 4 is sucked, the side wall 28b is deformed so as to be recessed radially inward, and the bottom portion 28a moves in the first direction D1. Thus, it is possible to relatively largely change a volume of the space surrounded by the partition wall main body 28. Due to this, the viscosity measurement can be performed without increasing a size of the fluid characteristic sensor 1EC.

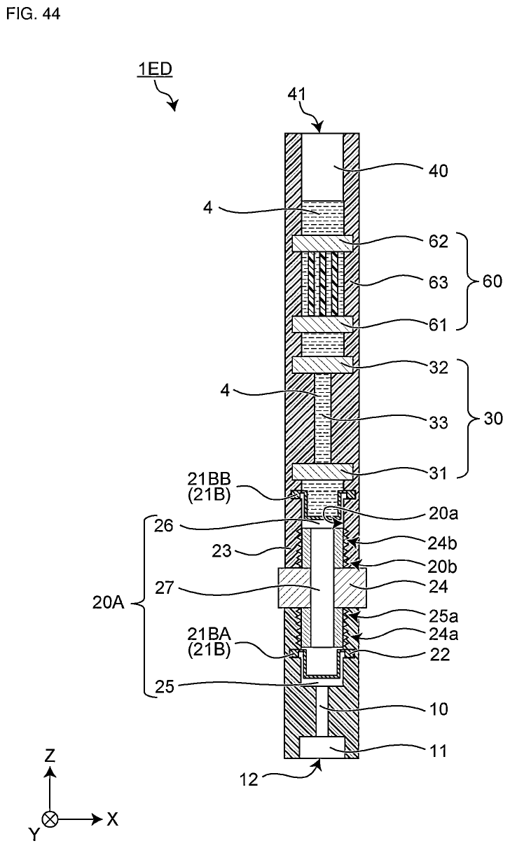

FIG. 44 is a schematic configuration diagram of an example of a fluid characteristic sensor according to Modification 8 of Preferred Embodiment 7 of the present invention. As illustrated in FIG. 44, the fluid characteristic sensor 1ED according to Modification 8 is provided by combining the configuration of the fluid characteristic sensor 1D according to Preferred Embodiment 6 with the solid partition wall 21B.

The fluid characteristic sensor 1ED includes a plurality of solid partition walls 21B. The plurality of solid partition walls 21B are individually disposed in the first connection flow path 25 and the second connection flow path 26 that define a portion of the first flow path 20A. Specifically, the plurality of solid partition walls 21B may include a first solid partition wall 21BA and a second solid partition wall 21BB. The first solid partition wall 21BA is disposed in the first connection flow path 25 of the attachment portion 22, and the second solid partition wall 21BB is disposed in the second connection flow path 26 of the main body 23.

The liquid 3 to be measured comes into contact with the first solid partition wall 21BA, but the working liquid 4 does not come into contact with the first solid partition wall 21BA. On the other hand, the working liquid 4 comes into contact with the second solid partition wall 21BB, but the liquid 3 does not come into contact with the second solid partition wall 21BB. The flow path between the first solid partition wall 21BA and the second solid partition wall 21BB is filled with a gas.

With such a configuration, since the liquid 3 to be measured does not enter into the main body 23, mixture of the working liquid 4 and the liquid 3 with each other can be reduced or prevented. In addition, since the attachment portion 22 can be easily replaced and used, usability for a user is improved. In addition, the plurality of solid partition walls 21B can reduce or prevent leakage of the working liquid 4. In addition, even when the fluid characteristic sensor 1ED is installed in an inclined manner, the liquid 3 and the working liquid 4 are not mixed with each other, and thus, it is possible to improve the flexibility of the installation location and the installation configuration of the fluid characteristic sensor 1ED.

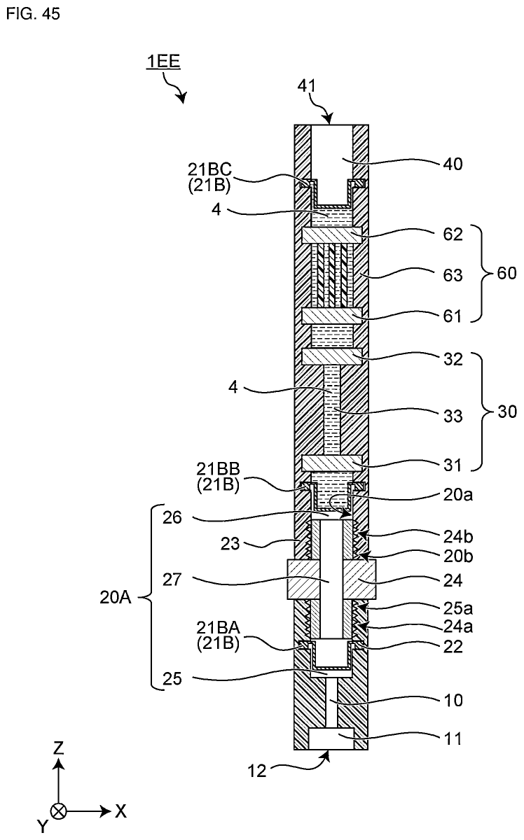

FIG. 45 is a schematic configuration diagram of an example of a fluid characteristic sensor according to Modification 9 of Preferred Embodiment 7 of the present invention. As illustrated in FIG. 45, a fluid characteristic sensor 1EE according to Modification 9 is provided by combining the configuration of the fluid characteristic sensor 1ED according to Modification 8 with a third solid partition wall 21BC.

In the fluid characteristic sensor 1EE, the plurality of solid partition walls 21B include the first solid partition wall 21BA, the second solid partition wall 21BB, and the third solid partition wall 21BC. The first solid partition wall 21BA is disposed in the first connection flow path 25 of the attachment portion 22, the second solid partition wall 21BB is disposed in the second connection flow path 26 of the main body 23, and the third solid partition wall 21BC is disposed in the working liquid flow path 40.

With such a configuration, leakage of the working liquid 4 from the open end 41 of the working liquid flow path 40 can be reduced or prevented. To be specific, the third solid partition wall 21BC seals the working liquid flow path 40 at the open end 41 side. Due to this, even when the fluid characteristic sensor 1EE is inclined or turned upside down, the leakage of the working liquid 4 can be reduced or prevented by the third solid partition wall 21BC.

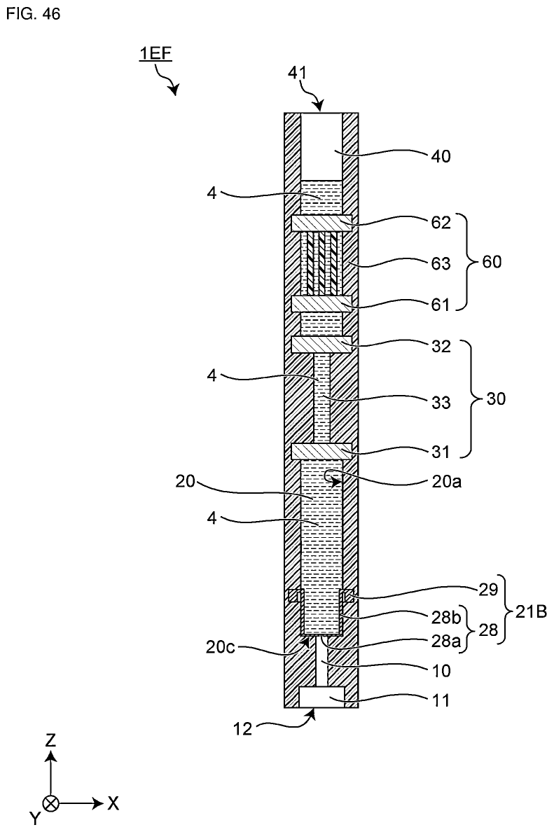

FIG. 46 is a schematic configuration diagram of an example of a fluid characteristic sensor according to Modification 10 of Preferred Embodiment 7 of the present invention. As illustrated in FIG. 46, the fluid characteristic sensor 1EF according to Modification 10 is different from the fluid characteristic sensor 1EC according to Modification 7 in that the solid partition wall 21B is disposed at a step 20c. Other configurations of the fluid characteristic sensor 1EF according to Modification 10 are the same as or similar to those of the fluid characteristic sensor 1EC according to Modification 7.

In the fluid characteristic sensor 1EF, the solid partition wall 21B is disposed at the step 20c. The step 20c is a portion where the pressure loss generator 10 and the first flow path 20 are connected to each other. A flow path diameter of the pressure loss generator 10 is smaller than a flow path diameter of the first flow path 20, which defines the step 20c at a portion where the pressure loss generator 10 and the first flow path 20 are connected to each other. The step 20c includes a surface extending in a direction intersecting the direction (Z direction) in which the first flow path 20 extends. In the fluid characteristic sensor 1EF, the step 20c includes a surface extending in a direction orthogonal or substantially orthogonal to the direction (Z direction) in which the first flow path 20 extends.

The bottom portion 28a of the solid partition wall 21B is in contact with the step 20c. In other words, the solid partition wall 21B is supported by the step 20c. This can reduce or prevent damage of the solid partition wall 21B. For example, when the working liquid 4 flows in the second direction D2 from the pump 60 toward the pressure loss generator 10, the step 20c supports the bottom portion 28a of the solid partition wall 21B. This can reduce or prevent deformation of the solid partition wall 21B beyond a strength limit. As a result, the damage of the solid partition wall 21B can be reduced or prevented.

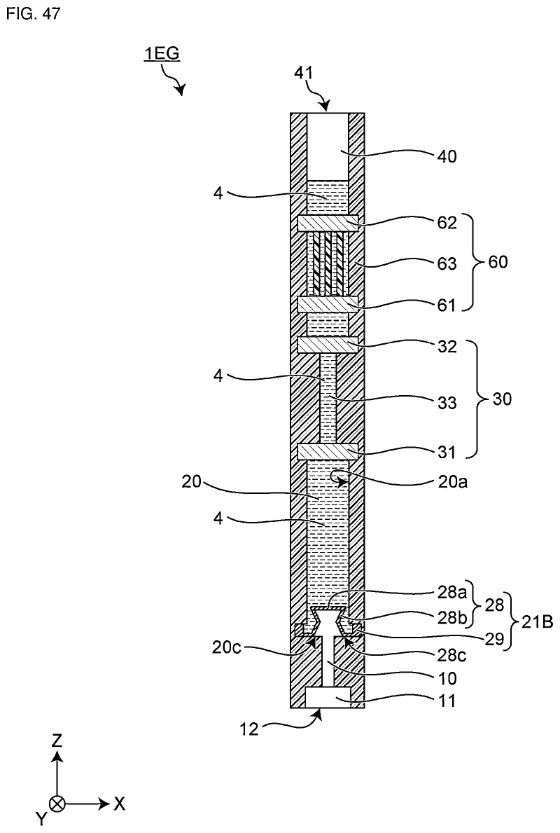

FIG. 47 is a schematic configuration diagram of an example of a fluid characteristic sensor according to Modification 11 of Preferred Embodiment 7 of the present invention. As illustrated in FIG. 47, a fluid characteristic sensor 1EG according to Modification 11 is different from the fluid characteristic sensor 1EF according to Modification 10 in that the solid partition wall 21B is disposed upside down. The other configurations of the fluid characteristic sensor 1EG according to Modification 11 are the same as or similar to those of the fluid characteristic sensor 1EF according to Modification 10.

In the fluid characteristic sensor 1EG, the open end 28c of the solid partition wall 21B is disposed at the step 20c. The open end 28c is an end portion provided with an opening, and is another end of the side wall 28b. The open end 28c of the solid partition wall 21B is disposed at the step 20c, and thus, the open end 28c is connected to the pressure loss generator 10. That is, the flow path of the pressure loss generator 10 communicates with the opening of the open end 28c.

In the fluid characteristic sensor 1EG, in a state before the liquid 3 to be measured is sucked in the first direction D1, the solid partition wall 21B is deformed so as to be recessed inward. By sucking the liquid 3 in the first direction D1, a force directed outward with respect to the partition wall main body 28 of the solid partition wall 21B is generated. Due to this, the side wall 28b of the solid partition wall 21B moves toward the inner wall 20a of the first flow path 20, and the bottom portion 28a moves in the first direction D1. The solid partition wall 21B is elastically deformed in this manner, which leads to the flow of the working liquid 4 in the first direction D1. Thus, the potential measurer 30 can measure the flow potential generated when the working liquid 4 flows.

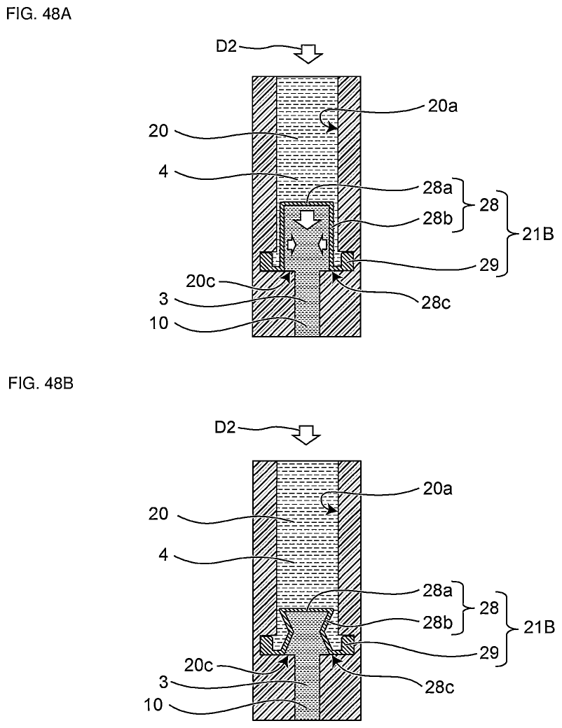

FIGS. 48A and 48B are schematic views illustrating an example of an operation of the solid partition wall according to Modification 11. FIGS. 48A and 48B illustrate an example of an operation of sucking the liquid 3 to be measured and then feeding the liquid 3 in the second direction D2. As illustrated in FIGS. 48A and 48B, when the liquid 3 is fed in the second direction D2, the solid partition wall 21B is elastically deformed. To be specific, by feeding the liquid 3 in the second direction D2, a force directed inward with respect to the partition wall main body 28 of the solid partition wall 21B is generated. Due to this, the side wall 28b of the partition wall main body 28 is elastically deformed so as to be recessed radially inward, and the bottom portion 28a of the partition wall main body 28 moves in the second direction D2.

In the fluid characteristic sensor 1EG, the open end 28c of the solid partition wall 21B is connected to the pressure loss generator 10, and the flow path of the pressure loss generator 10 is in communication with the opening of the open end 28c. For this reason, when the liquid 3 is fed in the second direction D2, even when the bottom portion 28a and the side wall 28b of the solid partition wall 21B are elastically deformed, the pressure loss generator 10 is hardly blocked. Thus, in the fluid characteristic sensor 1EG, when the liquid 3 is fed and discharged in the second direction D2, a remainder of the liquid 3 can be reduced or prevented in the first flow path 20.

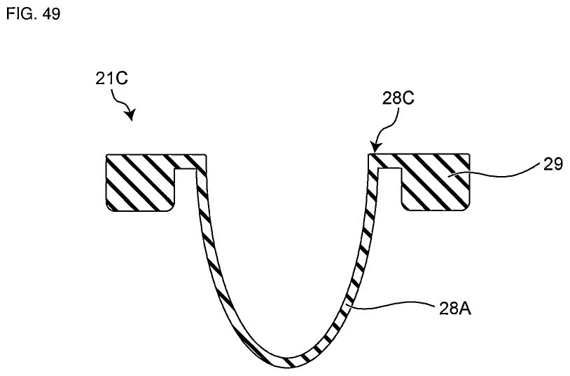

FIG. 49 is a schematic diagram illustrating another example of the solid partition wall. As illustrated in FIG. 49, the solid partition wall 21C may include a partition wall main body 28A recessed in a hemispherical shape and the flange 29. Also in such a configuration, the working liquid 4 flows in the first direction D1 due to elastic deformation of the partition wall main body 28A. Thus, the potential measurer 30 can measure the flow potential generated when the working liquid flows.

The shapes of the solid partition walls 21B and 21C are not limited to those in the above-described examples. The solid partition walls 21B and 21C may be recessed in a recessed shape, and may be an elastically deformable solid. In addition, it is preferable that the solid partition walls 21B and 21C have an elastically deformable shape or be made of an elastically deformable material so as to increase displacement in the flow direction of the working liquid 4.

### Preferred Embodiment 8

A fluid characteristic sensor according to Preferred Embodiment 8 of the present invention will be described. In Preferred Embodiment 8, differences from Preferred Embodiment 2 will be mainly described. In Preferred Embodiment 8, elements that are the same as or equivalent to those in Preferred Embodiment 2 are denoted by the same reference signs. Further, in Preferred Embodiment 8, description overlapping with that of Preferred Embodiment 2 will be omitted.

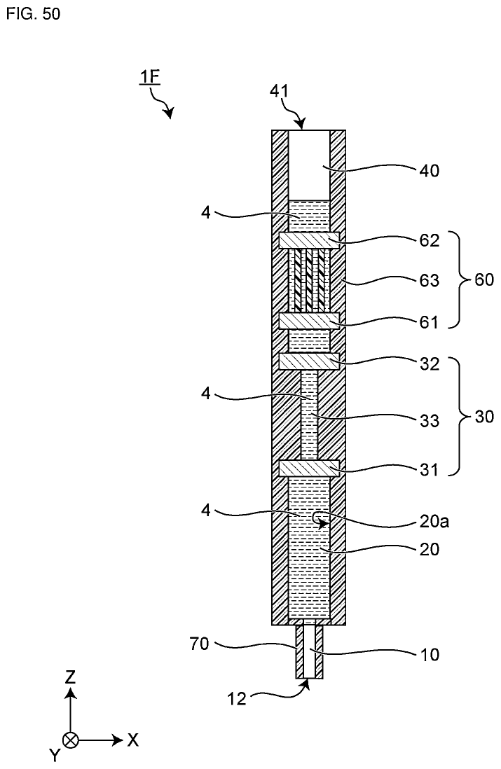

An example of the fluid characteristic sensor according to Preferred Embodiment 8 will be described with reference to FIG. 50. FIG. 50 is a schematic configuration diagram of the example of the fluid characteristic sensor according to Preferred Embodiment 8 of the present invention.

Preferred Embodiment 8 is different from Preferred Embodiment 2 in that a nozzle 70 is provided.

As illustrated in FIG. 50, a fluid characteristic sensor 1F according to Preferred Embodiment 8 includes the nozzle 70. The nozzle 70 includes the inflow/outflow port 12 and the pressure loss generator 10. The nozzle 70 has an outside diameter that is less than an outside diameter of the main body portion of the fluid characteristic sensor 1F.

### Advantageous Effects

According to the fluid characteristic sensor 1F according to Preferred Embodiment 8, the following advantageous effects can be obtained.

The fluid characteristic sensor 1F includes the nozzle 70 including the inflow/outflow port 12 and the pressure loss generator 10.

With such a configuration, it is possible to measure a fluid characteristic of a measurement target such as a small amount of liquid 3 like a liquid pool.

In Preferred Embodiment 8, an example in which the nozzle 70 includes the pressure loss generator 10 has been described, but the present invention is not limited thereto.

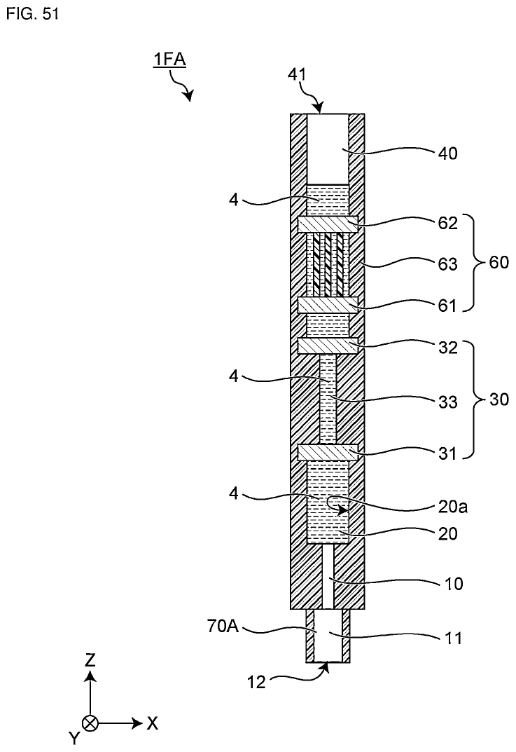

FIG. 51 is a schematic configuration diagram of an example of a fluid characteristic sensor according to Modification 10 of Preferred Embodiment 8 of the present invention. As illustrated in FIG. 51, in a fluid characteristic sensor 1FA according to Modification 10, a nozzle 70A includes the fluid flow path 11 and the inflow/outflow port 12, and the pressure loss generator 10 is provided in a main body portion of the fluid characteristic sensor 1FA. Even in such a configuration, it is possible to measure a fluid characteristic of a small amount of measurement target.

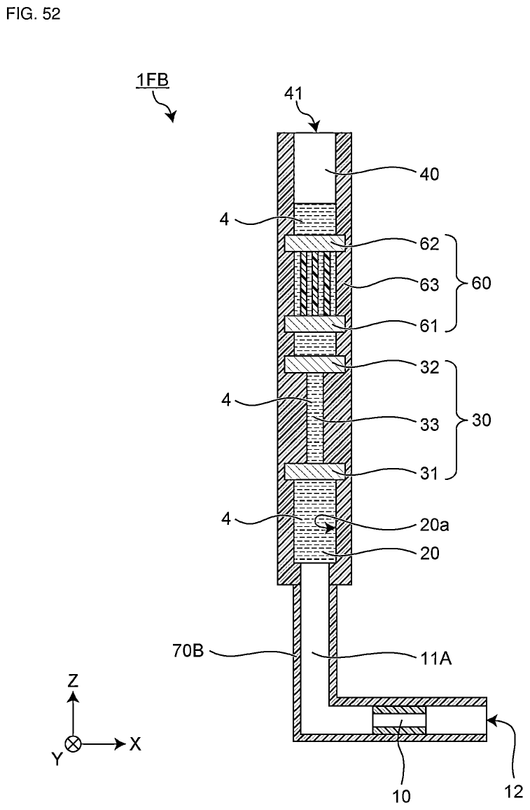

FIG. 52 is a schematic configuration diagram of an example of a fluid characteristic sensor according to Modification 11 of Preferred Embodiment 8 of the present invention. As illustrated in FIG. 52, in a fluid characteristic sensor 1FB according to Modification 11, a nozzle 70B includes the pressure loss generator 10, a fluid flow path 11A, and the inflow/outflow port 12. Also, the nozzle 70B is curved. With such a configuration, the nozzle 70B can be lengthened, and the main body portion of the fluid characteristic sensor 1FB can be installed at a position away from a measurement target.

### Other Preferred Embodiments

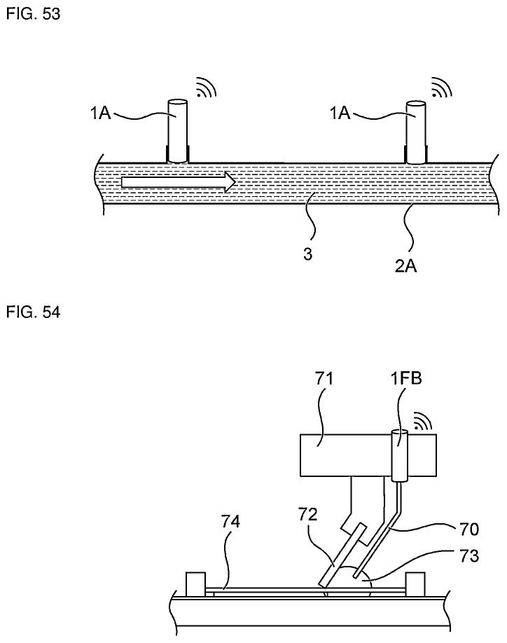

FIG. 53 is a schematic diagram illustrating another preferred embodiment. As illustrated in FIG. 53, a measurement system including a plurality of fluid characteristic sensors 1A may be constructed. The measurement system includes a plurality of fluid characteristic sensors 1A and a pipe 2A. A plurality of measurement holes are provided at the pipe 2A, and the fluid characteristic sensor 1A is installed in each of the plurality of measurement holes. In such a measurement system, by actively sucking and discharging the fluid in the pipe 2A, it is possible to automatically and continuously perform viscosity measurement and monitor variation in a fluid characteristic of the liquid 3 in the pipe 2A. Additionally, information about measurement results acquired by the plurality of fluid characteristic sensors 1A may be transmitted to a control device through wireless communication or wired communication. The above-described measurement system can be applied to a pipe or a tank in a food manufacturing process, a resin manufacturing process, an ink manufacturing process, a paste manufacturing process, or the like, for example, and can monitor the viscosity of a fluid flowing through the pipe. With this, a quality defect can be quickly detected, and a generation amount of defective products can be minimized. In the example illustrated in FIG. 53, an example of using the fluid characteristic sensor 1A according to Preferred Embodiment 1 has been described, but the present invention is not limited thereto. In the measurement system described above, the fluid characteristic sensors according to Preferred Embodiments 2 to 8 may be used.

FIG. 54 is a schematic diagram illustrating another preferred embodiment. As illustrated in FIG. 54, the fluid characteristic sensor 1FB may be installed in a printing apparatus 71. The fluid characteristic sensor 1FB may be installed at a squeegee 72 of the printing apparatus 71 to measure the viscosity of a liquid pool of a paste 73 accumulated in front of the squeegee 72 on a screen plate 74. In addition, the viscosity of the paste 73 in printing may be monitored to detect a variation in viscosity in real time. Due to this, it is possible to prevent printing failure caused by a change in viscosity of the paste 73 in advance. In addition, for example, the fluid characteristic sensor 1FB may be installed in, for example, a gravure printer, an inkjet printer, or a coating device such as a dispenser. For example, the fluid characteristic sensor 1FB may be used to prevent coating failure in advance or feedback control of a coating operation by detecting a change in viscosity of a coating liquid. In addition, the fluid characteristic sensor 1FB may be installed in a resin injection molding apparatus to be used for feedback control of an injection pressure based on detection of a change in viscosity of resin. The fluid characteristic sensors 1F and 1FA including the nozzle 70, other than the fluid characteristic sensor 1FB, may be used.

Although the present invention has been fully described in connection with preferred embodiments thereof with reference to the accompanying drawings, various modifications and alterations will become apparent to those skilled in the art. It is to be understood that such modifications and alterations are included in the present invention insofar as they do not depart from the scope of the present invention as defined by the appended claims.

### INDUSTRIAL APPLICABILITY

The fluid characteristic sensors according to preferred embodiments of the present invention are each a sensor to measure a characteristic of a fluid, and can be applied to, for example, a viscosity sensor.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A fluid characteristic sensor configured to measure a characteristic of a fluid to be measured, the fluid characteristic sensor comprising:
a pressure loss generator to generate a pressure loss when the fluid flows;
a first flow path connected to the pressure loss generator and through which the fluid and a working liquid that is a polar solvent flow;
a partition wall provided in the first flow path to partition the fluid and the working liquid from each other; and
a potential measurer connected to the first flow path to measure a flow potential generated when the working liquid flows.

2. The fluid characteristic sensor according to claim 1, wherein the pressure loss generator includes a thin tube with a flow path cross-sectional area smaller than a flow path cross-sectional area of the first flow path, or a porous body including a plurality of holes.

3. The fluid characteristic sensor according to claim 1, wherein the potential measurer includes:
a first electrode through which the working liquid is capable of passing;
a second electrode provided at an interval from the first electrode and through which the working liquid is capable of passing; and
a second flow path provided between the first electrode and the second electrode to be filled with the working liquid; and
the second flow path includes a thin tube with a flow path cross-sectional area smaller than a flow path cross-sectional area of the first flow path, or a porous body including a plurality of holes.

4. The fluid characteristic sensor according to claim 1, wherein the working liquid has at least one of a boiling point higher than a boiling point of water and a melting point lower than a melting point of water.

5. The fluid characteristic sensor according to claim 1, wherein
the partition wall includes a gas;
the first flow path extends in a gravity direction; and
in the first flow path, an interface between the working liquid and the partition wall is higher than an interface between the fluid and the partition wall.

6. The fluid characteristic sensor according to claim 5, wherein an inner wall of the first flow path has hydrophobicity.

7. The fluid characteristic sensor according to claim 1, further comprising a pump connected to the potential measurer to feed the working liquid.

8. The fluid characteristic sensor according to claim 7, wherein the pump is an electroosmotic flow pump, and includes:
a third electrode through which the working liquid is capable of passing;
a fourth electrode provided at an interval from the third electrode and through which the working liquid is capable of passing; and
a third flow path provided between the third electrode and the fourth electrode to be filled with the working liquid; and
the third flow path includes a porous body including a plurality of holes.

9. The fluid characteristic sensor according to claim 7, further comprising:
a pump controller configured or programmed to control a liquid feeding direction and a liquid feeding pressure of the pump; and
the liquid feeding direction is a first direction from the pressure loss generator toward the pump, and a second direction opposite to the first direction, the second direction extending from the pump toward the pressure loss generator.

10. The fluid characteristic sensor according to claim 9, wherein the pump controller is configured or programmed to control the liquid feeding direction of the pump based on a measurement value of the flow potential measured by the potential measurer.

11. The fluid characteristic sensor according to claim 10, wherein the pump controller is configured or programmed to:
switch the liquid feeding direction to the second direction after the measurement value of the flow potential converges when the liquid feeding direction is the first direction; and
stop the pump when the liquid feeding direction is the second direction and an absolute value of a change amount in flow potential per unit time increases beyond a threshold value.

12. The fluid characteristic sensor according to claim 10, wherein
the partition wall has a volume larger than a flow path volume of the pressure loss generator; and
the pump controller is configured or programmed to stop the pump when the liquid feeding direction is the second direction and an absolute value of a change amount in measurement value of the flow potential per unit time decreases beyond a predetermined threshold value.

13. The fluid characteristic sensor according to claim 9, wherein the pump controller is configured or programmed to change the liquid feeding pressure in a stepwise manner.

14. The fluid characteristic sensor according to claim 1, further comprising a calculator to calculate a characteristic of the fluid based on the flow potential measured by the potential measurer.

15. The fluid characteristic sensor according to claim 9, further comprising:
a calculator to calculate a characteristic of the fluid based on the flow potential measured by the potential measurer;
the calculator is configured to:
calculate a first viscosity of the fluid based on a measurement value of the flow potential when the liquid feeding direction is the first direction;
calculate a second viscosity of the fluid based on a measurement value of the flow potential when the liquid feeding direction is the second direction; and
determine the characteristic of the fluid based on the first viscosity and the second viscosity.

16. The fluid characteristic sensor according to claim 15, wherein the pump controller is configured or programmed to:
set the liquid feeding pressure of the pump when the liquid feeding direction is the first direction to a first pressure; and
set the liquid feeding pressure of the pump when the liquid feeding direction is the second direction to a second pressure different from the first pressure.

17. The fluid characteristic sensor according to claim 14, wherein the calculator is configured to:
calculate a flow velocity of the working liquid based on the flow potential measured by the potential measurer; and
calculate a viscosity of the fluid based on the flow velocity of the working liquid.

18. The fluid characteristic sensor according to claim 1, wherein
a working liquid flow path that includes an open end opened to an atmosphere side and through which the working liquid flows; and
a liquid surface of the working liquid at a side of the open end of the working liquid flow path is covered with a non-polar solvent.

19. The fluid characteristic sensor according to claim 18, wherein a boiling point of the non-polar solvent is higher than a boiling point of the working liquid.

20. The fluid characteristic sensor according to claim 18, wherein the non-polar solvent is a non-volatile solvent.

21. The fluid characteristic sensor according to claim 1, further comprising:
an attachment portion including an inflow/outflow port through which the fluid flows in and out and the pressure loss generator; and
a main body including at least a portion of the first flow path; wherein
the main body detachably attachable to the attachment portion.

22. The fluid characteristic sensor according to claim 1, wherein the partition wall is a solid.

23. The fluid characteristic sensor according to claim 22, wherein the partition wall includes:
a partition wall main body elastically deformable and having a recessed shape; and
a flange protruding outward from an outer wall of the partition wall main body.

24. The fluid characteristic sensor according to claim 1, wherein the partition wall includes a plurality of the partition walls.

25. A fluid characteristic sensor configured to measure a characteristic of a fluid to be measured, the fluid characteristic sensor comprising:
a first flow path through which the fluid and a working liquid that is a polar solvent flow, the first flow path including one end and another end;
a partition wall provided in the first flow path to partition the fluid and the working liquid from each other;
a pressure loss generator connected to a side of the one end of the first flow path and including a flow path cross-sectional area smaller than a flow path cross-sectional area of the first flow path; and
a potential measurer connected to a side of the another end of the first flow path to measure a flow potential generated when the working liquid flows.
